(19) United States
(12) Patent Application Publication
(10) Pub. No.: US 2023/0008367 A1
(43) Pub. Date: Jan. 12, 2023

(54) COMPOUNDS USEFUL FOR TREATING LIVER DISEASES

(71) Applicant: ABIONYX PHARMA SA, Balma (FR)

(72) Inventor: Jean-Louis Henri Dasseux, Toulouse (FR)

(73) Assignee: ABIONYX PHARMA SA, Balma (FR)

(21) Appl. No.: 17/763,731

(22) PCT Filed: Sep. 25, 2020
(86) PCT No.: PCT/IB2020/000808

§ 371 Date: Mar. 25, 2022

Related U.S. Application Data
(60) Provisional application No. 62/906,288, filed on Sep. 26, 2019.

(51) Int. Cl. A61K 31/192; C07C 323/22; A61P 3/10

(52) U.S. Cl. CPC A61K 31/192; C07C 323/22; A61P 3/10

(57) ABSTRACT

This invention provides compounds of Formulae (A) and (B) and pharmaceutically acceptable salts, solvates, esters, amides, and prodrugs thereof, including 2-(4-(3-hydroxy-3-(4-(methylthio)phenyl)prop-1-en-1-yl)-2,6-dimethylphenoxy)-2-methylpropanoic acid (“Compound I”), 3-(4-((1-hydroxy-2-methylpropan-2-yl)oxy)-3,5-dimethylphenyl)-1-(4-(methylthio)phenyl)prop-2-en-1-one (“Compound II”) and 3-(4-((1-hydroxy-2-methylpropan-2-yl)oxy)-3,5-dimethylphenyl)-1-(4-(methylthio)phenyl)prop-2-en-1-ol (“Compound III”), and pharmaceutically acceptable salts, solvates, esters, amides, and prodrugs thereof. The invention further provides pharmaceutical compositions comprising a compound of Formulae (A) and (B) or a pharmaceutically acceptable salt, solvate, ester, amide, or prodrug thereof, including Compound I, Compound II, or Compound III, or a pharmaceutically acceptable salt, solvate, ester, amide, or prodrug thereof, and a pharmaceutically acceptable carrier or vehicle. The compounds and compositions disclosed herein are useful for treating or preventing liver disease such as liver fibrosis, fatty liver disease, non-alcoholic fatty liver disease (NAFLD) or non-alcoholic steatohepatitis (NASH).

### CROSS-REFERENCE TO RELATED APPLICATIONS

This application claims the priority benefit of U.S. provisional application No. 62/906,288, filed Sep. 26, 2019, the contents of which are incorporated herein in their entireties by reference thereto.

### FIELD OF THE INVENTION

This invention provides compounds of Formulae (A) and (B) and pharmaceutically acceptable salts, solvates, esters, amides, and prodrugs thereof, such as 2-(4-(3-hydroxy-3-(4-(methylthio)phenyl)prop-1-en-1-yl)-2,6-dimethylphenoxy)-2-methylpropanoic acid (“Compound I”), 3-(4-((1-hydroxy-2-methylpropan-2-yl)oxy)-3,5-dimethylphenyl)-1-(4-(methylthio)phenyl)prop-2-en-1-one (“Compound II”), and 3-(4-((1-hydroxy-2-methylpropan-2-yl)oxy)-3,5-dimethylphenyl)-1-(4-(methylthio)phenyl)prop-2-en-1-ol (“Compound III”), and pharmaceutically acceptable salts, solvates, esters, amides, and prodrugs thereof. The invention further provides pharmaceutical compositions comprising a compound of Formulae (A) and (B) or a pharmaceutically acceptable salt, solvate, ester, amide, or prodrug thereof, such as Compound I, Compound II or Compound III, or a pharmaceutically acceptable salt, solvate, ester, amide, or prodrug thereof, and a pharmaceutically acceptable carrier or vehicle. The compounds and compositions disclosed herein are useful for treating or preventing liver disease such as liver fibrosis, fatty liver disease, non-alcoholic fatty liver disease (NAFLD) or non-alcoholic steatohepatitis (NASH).

### BACKGROUND

Elevated levels of low-density lipoprotein cholesterol (LDL-C) and triglycerides are associated with mixed dyslipidemia. Type IIb hyperlipidemia, a type of mixed dyslipidemia, is characterized by elevation of apolipoprotein B, very low-density lipoprotein cholesterol (VLDL-C), intermediate density lipoprotein cholesterol (IDL), and small dense low-density lipoprotein (LDL) levels, in addition to elevation in LDL-C and triglyceride levels.

Liver diseases, such as a non-alcoholic fatty liver disease (NAFLD) comprise a spectrum of conditions ranging from relatively benign steatosis to more severe non-alcoholic steatohepatitis (NASH), the latter of which, if untreated, can lead to fibrosis, cirrhosis, liver failure, or hepatocellular carcinoma. NAFLD and NASH can develop due to hepatic triglyceride overproduction and accumulation. NAFLD is strongly associated with features of obesity, diabetes, dyslipidemia, hyperlipidemia and metabolic syndrome, including obesity, insulin resistance, type-2 diabetes mellitus, and dyslipidemia. NASH can cause the liver to swell, become inflamed, become fibrotic, become damaged and become ultimately less functional. NASH tends to develop in people who are overweight or obese, or have diabetes, mixed dyslipidemia, high cholesterol or high triglycerides or an inflammatory condition. NASH is marked by hepatocyte ballooning and liver inflammation, which can lead to liver damage and progress to scarring and irreversible changes, similar to the damage caused by heavy alcohol use.

Liver steatosis and fibrosis can also be induced by drugs, such as amiodarone, valproate, tamoxifen, methotrexate, and some chemotherapeutic and antiretroviral agents (Amacher, D. E., et al. Semin. Liver Dis., 2014, 34, 205). Drug-induced hepatic steatosis can be reversible and may involve drug accumulation in the liver.

NAFLD, NASH, fatty liver, or drug-induced liver steatosis can lead to metabolic complications including elevation of liver enzymes, fibrosis, cirrhosis, hepatocellular carcinoma, and liver failure. Liver failure is life-threatening and therefore there is a need to develop therapies to delay development, prevent formation or reverse the condition of a fatty liver.

Peroxisome proliferator-activated receptors (PPARs) have been identified as targets for the treatment of cardiometabolic diseases including diabetes, insulin resistance, dyslipidemia, and liver diseases such as NAFLD and NASH. There are three types of PPARs: PPARα, PPARγ and PPARδ. Several PPAR agonists have been marketed, including fenofibrate (a PPARα agonist), bezafibrate (a PPAR pan agonist), pioglitazone (a PPARγ agonist), and rosiglitazone (a PPARγ agonist). Recently, PPAR agonists such as seladelpar (a PPARδ agonist), lanifibranor (a pan agonist), and elafibranor (a dual PPARα/δ agonist) have been studied for the treatment of NASH and primary biliary cholangitis (PBC). However, several clinical trials involving such PPAR agonists have failed due to toxicity or failure to meet primary endpoint. For example, in a Phase 3 trial in adults with NASH and fibrosis, elafibranor did not demonstrate a statistically significant effect on the primary endpoint of NASH resolution without worsening of fibrosis (ir.genfit.com/news-releases/news-release-details/genfit-announces-results-interim-analysis-resolve-it-phase-3).

There remains a need for new preventions and treatments for liver disorders and other conditions associated with PPARs.

### SUMMARY OF THE INVENTION

The present invention provides novel compounds and their use to treat various disorders, for example, liver disorders such as NASH and other conditions associated with PPARs. Without being bound by theory, the inventor believes that the clinical usefulness of PPAR agonists such as elafibranor are limited by their toxicity such that doses often cannot be increased sufficiently to reach an effective dose. The subject invention provides novel compounds, including derivatives of elafibranor and related compounds. Without being bound by theory, the inventor believes that the compounds described herein can act as PPAR agonists and/or as PPAR agonist prodrugs, which have advantageous properties that result in improved bioavailability and/or half-life and/or safety and/or efficacy and/or improved therapeutic indexes, following administration. In particular, the compound may thus have an improved therapeutic index. The therapeutic index (TI) is a ratio that compares the dose at which a compound becomes toxic against the dose at which it is effective. One common measure of TI is TD₅₀/ED₅₀, wherein TD₅₀ and ED₅₀ are the toxic and effective doses, respectively, for 50% of the population. The larger the TI, the safer a compound is. Compounds with a low TI can be difficult to use in clinical practice and often require monitoring of plasma concentration in order to prevent toxicity. The one or more advantageous properties of the compounds of the disclosure (compared to known PPAR agonists such as elafibranor) can include, for example, better solubility, better kinetics, better absorption, better PPAR receptor selectivity at pharmaceutically effective doses, reduced drug metabolism by cytochrome P450 or other enzymes such as reductases, reduced glucuronidation, reduced toxicity, or a combination thereof.

In one aspect, the present invention provides compounds of Formula (A):

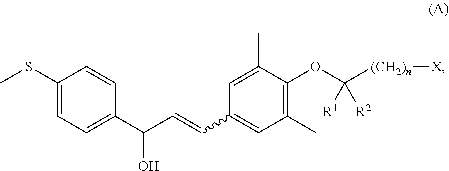

and pharmaceutically acceptable salts, solvates, esters, amides, and prodrugs thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COOH, —COH, —COOR³, —COOCH₂CONR⁴R⁵, —SO₃H,

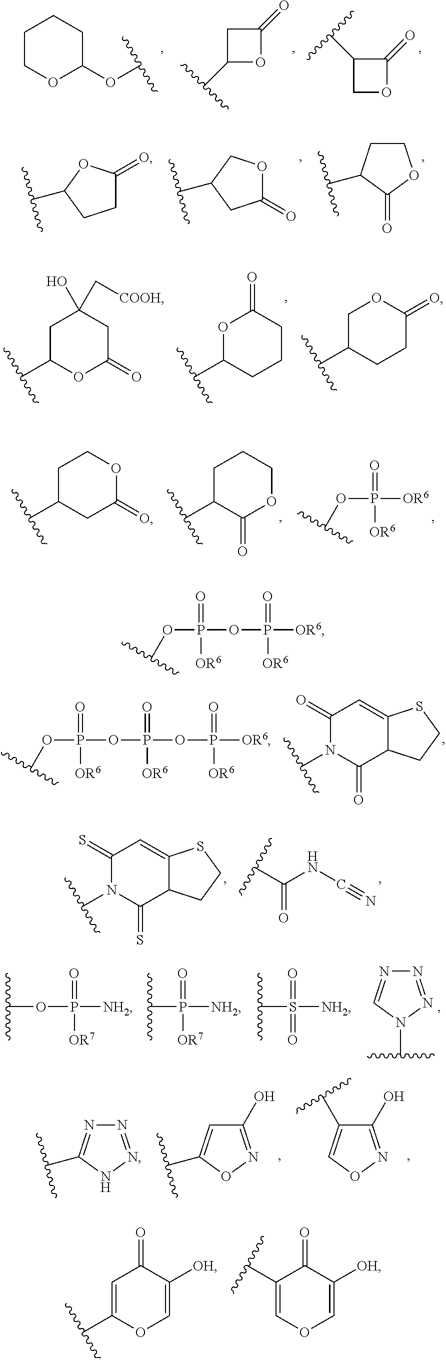

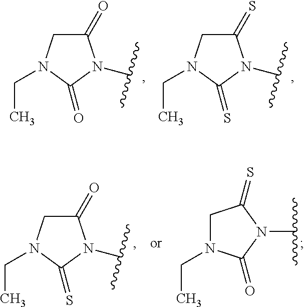

R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

In another aspect, the present invention also provides compounds of Formula (B):

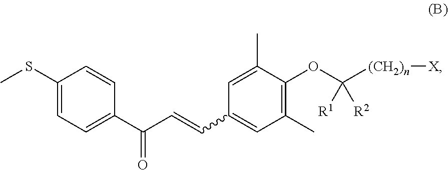

and pharmaceutically acceptable salts, solvates, esters, amides, and prodrugs thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COH, —COOCH₂CONR⁴R⁵, —SO₃H,

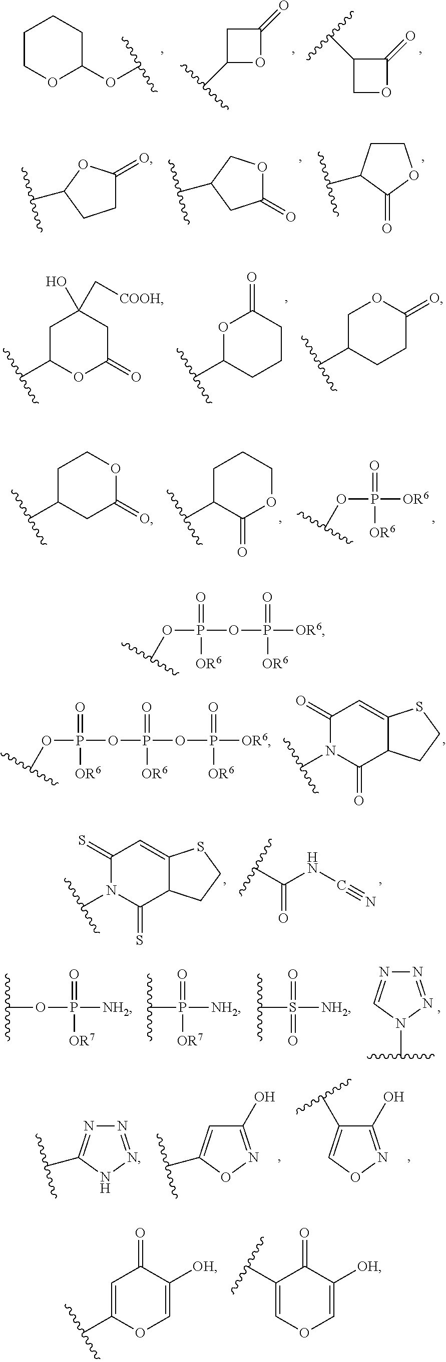

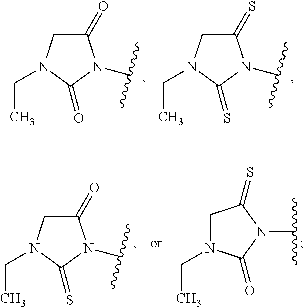

R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

The present invention provides 2-(4-(3-hydroxy-3-(4-(methylthio)phenyl)prop-1-en-1-yl)-2,6-dimethylphenoxy)-2-methylpropanoic acid (“Compound I”) and pharmaceutically acceptable salts, solvates, esters, amides, and prodrugs, thereof, wherein Compound I has the structure:

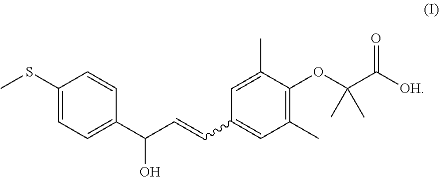 [SMILES: CSc1ccc(C(O)C=Cc2cc(C)c(OC(C)(C)C(=O)O)c(C)c2)cc1]

The present invention also provides 3-(4-((1-hydroxy-2-methylpropan-2-yl)oxy)-3,5-dimethylphenyl)-1-(4-(methylthio)phenyl)prop-2-en-1-one (“Compound II”) and pharmaceutically acceptable salts, solvates, esters, amides, and prodrugs thereof, wherein Compound II has the structure:

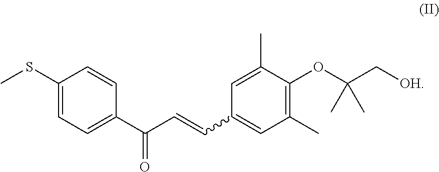 [SMILES: CSc1ccc(C(=O)C=Cc2cc(C)c(OC(C)(C)CO)c(C)c2)cc1]

The present invention further provides 3-(4-((1-hydroxy-2-methylpropan-2-yl)oxy)-3,5-dimethylphenyl)-1-(4-(methylthio)phenyl)prop-2-en-1-ol (“Compound III”) and pharmaceutically acceptable salts, solvates, esters, amides, and prodrugs thereof, wherein Compound III has the structure

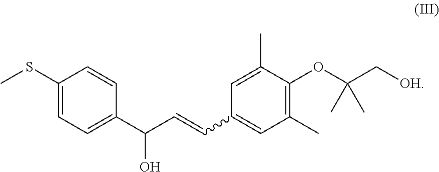 [SMILES: CSc1ccc(C(O)C=Cc2cc(C)c(OC(C)(C)CO)c(C)c2)cc1]

Each compound of Formula (A) and Formula (B), and each compound of Compounds I, II, and III, or a pharmaceutically acceptable salt, solvate, ester, amide, and prodrug thereof is a “compound of the invention”.

The present invention also provides compositions comprising i) an effective amount of a compound of the invention and ii) a pharmaceutically acceptable carrier or vehicle (each composition being a “composition of the invention”).

The present invention further provides methods for treating or preventing a liver disorder, dyslipidemia, dyslipoproteinemia, a renal disease, a disorder of glucose metabolism, a disorder of lipid metabolism, a disorder of glucid metabolism, a cardiovascular disease, a vascular disease, a metabolic syndrome, a complication associated with metabolic syndrome, a PPAR-associated disorder, septicemia, a thrombotic disorder, obesity, diabetic nephropathy, diabetic retinopathy, atherosclerosis, pancreatitis, a cerebrovascular disease, a disorder related to neovascularization, hypertension, cancer, inflammation, an inflammatory disease, a neurodegenerative disease, an autoimmune disease, a neoplastic disease, muscle atrophy, cholestasis, mitochondrial dysfunction, an ocular disease, a lysosomal storage disease, a kidney disease, or impotence, comprising administering to a subject in need thereof an effective amount of a compound of the invention or a composition of the invention.

The present invention further provides methods for treating or preventing hyperlipemia, hyperlipidemia, hyperlipoproteinemia, hypercholesterolemia, hypertriglyceridemia, or dyslipidemia, comprising administering to a subject in need thereof an effective amount of a compound of the invention or a composition of the invention.

The present invention further provides methods for treating a subject having or preventing a subject from having an abnormally high concentration in a subject's blood plasma or blood serum of high low-density lipoprotein (LDL), apolipoprotein B (apo B), lipoprotein(a) (Lp(a)), apolipoprotein (a), or very low-density lipoprotein (VLDL), comprising administering to a subject in need thereof an effective amount of a compound of the invention or a composition of the invention.

The present invention further provides methods for treating a subject having or preventing a subject from having an abnormally low concentration in a subject's blood plasma or blood serum of high-density lipoprotein (HDL), comprising administering to a subject in need thereof an effective amount of the compound of the invention or the composition of the invention.

The present invention further provides methods for treating a subject having or preventing a subject from having an abnormally reduced or deficient lipoprotein lipase concentration or activity in a subject's blood plasma or blood serum, comprising administering to a subject in need thereof an effective amount of a compound of the invention or a composition of the invention.

The present invention provides methods for treating or preventing hypoalphalipoproteinemia, a lipoprotein abnormality associated with diabetes, a lipoprotein abnormality associated with obesity, a lipoprotein abnormality associated with Alzheimer's Disease, or familial combined hyperlipidemia, comprising administering to a subject in need thereof an effective amount of a compound of the invention or a composition of the invention.

The present invention further provides methods for reducing in a subject's blood plasma or blood serum an abnormally high concentration of triglycerides, low-density lipoprotein cholesterol (LDL-C), very low-density lipoprotein cholesterol (VLDL-C), non-high-density lipoprotein cholesterol, (non-HDL-C), lipoprotein(a) (Lp(a)), apolipoprotein B, HDL/(VLDL+LDL) ratio, apolipoprotein C-II or apolipoprotein C-III, comprising administering to a subject in need thereof an effective amount of a compound of the invention or a composition of the invention.

The present invention further provides methods for elevating in a subject's blood plasma or blood serum an abnormally low concentration of a high-density lipoprotein (HDL) associated protein, HDL-cholesterol, apolipoprotein A-I, or apolipoprotein E, comprising administering to a subject in need thereof an effective amount of a compound of the invention or a composition of the invention.

The present invention further provides methods for promoting clearance of triglycerides from a subject's blood plasma or blood serum, comprising administering to a subject in need thereof an effective amount of a compound of the invention or a composition of the invention.

The present invention further provides methods for increasing abnormally low glucose metabolism or increasing abnormally low lipid metabolism in a subject, comprising administering to a subject in need thereof an effective amount of a compound of the invention or a composition of the invention.

The present invention further provides methods for treating or preventing one or more symptoms of inflammation, systemic lupus erythematosus, lupus nephritis, or arthritis, comprising administering to a subject in need thereof an effective amount of a compound of the invention or a composition of the invention.

The present invention further provides methods for reducing the fat content of meat in livestock, comprising administering to livestock an effective amount of a compound of the invention or a composition of the invention.

The present invention further provides methods for reducing cholesterol content of a fowl egg, comprising administering to a fowl species an effective amount of a compound of the invention or a composition of the invention.

### DETAILED DESCRIPTION OF THE INVENTION

### Definitions

The term “about” when immediately preceding a numerical value means ± up to 20% of the numerical value. For example, “about” a numerical value means ± up to 20% of the numerical value, in some embodiments, ± up to 19%, up to 18%, ± up to 17%, ± up to 16%, up to 15%, up to 14%, ± up to 13%, ± up to 12%, up to 11%, ± up to 10%, ± up to 9%, ± up to 8%, ± up to 7%, ± up to 6%, up to 5%, ± up to 4%, ± up to 3%, ± up to 2%, ± up to 1%, ±up to less than 1%, or any other value or range of values therein.

Throughout the present specification, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “pharmaceutically acceptable salt” includes both an acid and a base addition salt. Pharmaceutically acceptable salts can be obtained by reacting the compound of the invention functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Pharmaceutically acceptable salts can also be obtained by reacting a compound of the invention functioning as an acid, with an inorganic or organic base to form a salt, for example, salts of sodium, potassium, lithium, ammonium, calcium, magnesium, iron, zinc, copper, manganese, aluminum, ammonia, isopropylamine, trimethylamine, choline, betaine, etc. Inorganic base can include, but are not limited to, calcium hydroxide, potassium hydroxide, sodium hydroxide, and sodium carbonate. Organic base can include, but are not limited to, primary amines, secondary amines, tertiary amines, substituted amines including naturally-occurring substituted amines, and cyclic amines, such as isopropylamine, trimethylamine, diethylamine, triethylamine, tripropylamine, ethanolamine, 2-dimethylaminoethanol, tromethamine, lysine, arginine, histidine, caffeine, procaine, hydrabamine, glucoasamine, N-alkylgucamines, theobromine, purines, piperazine, piperidine, N-ethylpiperidine, and the like. Those skilled in the art will further recognize that pharmaceutically acceptable salts can be prepared by reaction of the compounds of the invention with an appropriate inorganic or organic acid or base via any of a number of known methods.

The term “solvate” refers to a solvation complex. Solvates can be formed by solvation (the combination of solvent molecules with molecules or ions of the compounds of the invention), or a solvate can be an aggregate that comprises a solute ion or molecule or a solvent molecules. The solvent can be water, in which case the solvate is a hydrate. Examples of hydrates include, but are not limited to, a hemihydrate, monohydrate, dihydrate, trihydrate, hexahydrate, etc. The solvate can be formed via hydration, including via absorption of moisture. A pharmaceutically acceptable salt can also be a solvate. Where a solvate is obtained via crystallization from a solvent, the solvent can be an alcohol, such as methanol or ethanol; an aldehyde; a ketone, such as acetone; or an ester, such as ethyl acetate.

The compounds of the invention can have one or more asymmetric centers and can thus be enantiomers, racemates, diastereomers, other stereoisomers and mixtures thereof. The compounds of the invention include all such possible isomers (including geometric isomers), as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation or isolation of individual enantiomers include chiral synthesis from a suitable optically pure precursor or resolution of the racemate using, for example, chiral high pressure liquid chromatography (HPLC). Likewise, the compounds of the invention include all tautomeric forms.

The language “substantially free of its corresponding opposite enantiomer” means having no more than about 10 mol %, in another embodiment no more than about 5 mol %, in another embodiment no more than about 2 mol %, in another embodiment no more than about 1 mol %, in another embodiment no more than about 0.5 mol % and in another embodiment no more than about 0.1 mol %, of its corresponding opposite enantiomer.

The language “substantially free of its corresponding opposite stereoisomer” means having no more than about 10 mol %, in another embodiment no more than about 5 mol %, in another embodiment no more than about 2 mol %, in another embodiment no more than about 1 mol %, in another embodiment no more than about 0.5 mol % and in another embodiment no more than about 0.1 mol %, of its corresponding opposite stereoisomer.

The language “substantially free of its corresponding other olefin configuration” means having no more than about 10 mol %, in another embodiment no more than about 5 mol %, in another embodiment no more than about 2 mol %, in another embodiment no more than about 1 mol %, in another embodiment no more than about 0.5 mol % and in another embodiment no more than about 0.1 mol %, of its corresponding other olefin configuration.

An “effective amount” when used in connection with a compound of the invention means an amount of the compound of the invention that, when administered to a subject is effective to treat or prevent a disorder or condition disclosed herein, alone or with another pharmaceutically active agent.

An “effective amount” when used in connection with another pharmaceutically active agent means an amount of the other pharmaceutically active agent that is effective to treat or prevent a disorder or condition disclosed herein, alone or in combination with a compound of the invention.

A “subject” is a human or non-human mammal, e.g., a bovine, horse, feline, canine, rodent, or non-human primate. The human can be a male or female, child, adolescent or adult. The female can be premenarcheal or postmenarcheal.

“Mammal” includes a human, domestic animal such as a laboratory animal (e.g., mouse, rat, rabbit, monkey, dog, etc.) and household pet (e.g., cat, dog, swine, cattle, sheep, goat, horse, rabbit), and a non-domestic, wild animal.

All weight percentages (i.e., “% by weight” and “wt. %” and w/w) referenced herein, unless otherwise indicated, are relative to the total weight of the mixture or composition, as the case can be.

“Alkyl” refers to a fully saturated, straight or branched hydrocarbon chain having from one to twelve carbon atoms, and which is attached to an atom by a single bond. Alkyls with a number of carbon atoms ranging from 1 to 12 are included. An alkyl group with 1 to 12 carbon atoms is a C₁-C₁₂ alkyl, an alkyl group with 1 to 10 carbon atoms is a C₁-C₁₀ alkyl, an alkyl group with 1 to 6 carbon atoms is a C₁-C₆ alkyl and an alkyl group with 1 to 5 carbon atoms is a C₁-C₅ alkyl. A C₁-C₅ alkyl includes C₅ alkyls, C₄ alkyls, C₃ alkyls, C₂ alkyls and C, alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise, an alkyl group can be unsubstituted or substituted with a substituent disclosed herein.

“Alkenyl” refers to a straight or branched hydrocarbon chain having from two to twelve carbon atoms, and having one or more carbon-carbon double bonds. Each alkenyl group is attached to an atom by a single bond. Alkenyl groups with a number of carbon atoms ranging from 2 to 12 are included. An alkenyl group with 2 to 12 carbon atoms is a C₂-C₁₂ alkenyl, an alkenyl group with 2 to 10 carbon atoms is a C₂-C₁₀ alkenyl, an alkenyl group with 2 to 6 carbon atoms is a C₂-C₆ alkenyl and an alkenyl group with 2 to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise, an alkyl group can be unsubstituted or substituted with a substituent disclosed herein.

“Alkynyl” refers to a straight or branched hydrocarbon chain radical having from two to twelve carbon atoms, and having one or more carbon-carbon triple bonds. Each alkynyl group is attached to an atom by a single bond. Alkynyl groups with a number of carbon atoms ranging from 2 to 12 are included. An alkynyl group having 2 to 12 carbon atoms is a C₂-C₁₂ alkynyl, an alkynyl group with 2 to 10 carbon atoms is a C₂-C₁₀ alkynyl, an alkynyl group with 2 to 6 carbon atoms is a C₂-C₆ alkynyl and an alkynyl group with 2 to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes Cr alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise, an alkyl group can be unsubstituted or substituted with a substituent disclosed herein.

“Aryl” refers to a hydrocarbon ring system radical comprising hydrogen, 6 to 18 carbon atoms and at least one aromatic ring. The aryl radical can be a monocyclic, bicyclic, tricyclic or tetracyclic ring system, which can include fused or bridged ring systems. Aryl radicals include, but are not limited to, aceanthrylenyl, acenaphthylenyl, acephenanthrylenyl, anthracenyl, azulenyl, chrysenyl, fluoranthenyl, fluorenyl, as-indacenyl, s-indacenyl, indanyl, indenyl, naphthalenyl, phenalenyl, phenanthrenyl, phenyl, pleiadenyl, pyrenyl, and triphenylenyl. Unless stated otherwise, the aryl can be unsubstituted or substituted with a substituent disclosed herein.

“Cycloalkyl” refers to a non-aromatic monocyclic or polycyclic fully saturated hydrocarbon radical consisting of carbon and hydrogen atoms, which can include fused or bridged ring systems, having from three to twenty carbon atoms, preferably having from three to ten carbon atoms, and which is attached to an atom by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless stated otherwise, a cycloalkyl group can be unsubstituted or substituted with a substituent disclosed herein.

“Heteroaryl” refers to a 5- to 20-membered ring system radical including hydrogen atoms, one to thirteen carbon atoms, one to six nitrogen, oxygen or sulfur heteroatoms, and at least one aromatic ring. The heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples of heteroaryl include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thienyl). Unless stated otherwise, a heteroaryl group can be unsubstituted or substituted.

“Heterocyclyl” refers to a 3- to 20-membered non-aromatic, partially unsaturated, or aromatic ring radical which includes two to twelve carbon atoms and from one to six nitrogen, oxygen or sulfur heteroatoms. Heterocycyl include heteroaryls as defined herein. Unless stated otherwise, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise, a heterocyclyl group can be unsubstituted or substituted with a substituent disclosed herein.

As used herein, the symbol

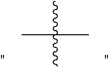

(a “point of attachment bond”) denotes a bond that is a point of attachment between two chemical entities, one of which is depicted as being attached to the point of attachment bond and the other of which is not depicted as being attached to the point of attachment bond. For example,

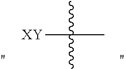

indicates that the chemical entity “XY” is bonded to another chemical entity via the point of attachment bond.

### The Compounds of the Invention

### Compounds of Formula (A)

In some embodiments, the compound of the invention is a compound of Formula (A):

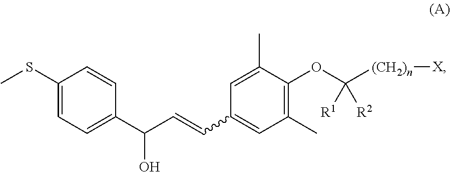

or a pharmaceutically acceptable salt, solvate, ester, amide, or prodrug thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COOH, —COH, —COOR³, —COOCH₂CONR⁴R⁵, —SO₃H,

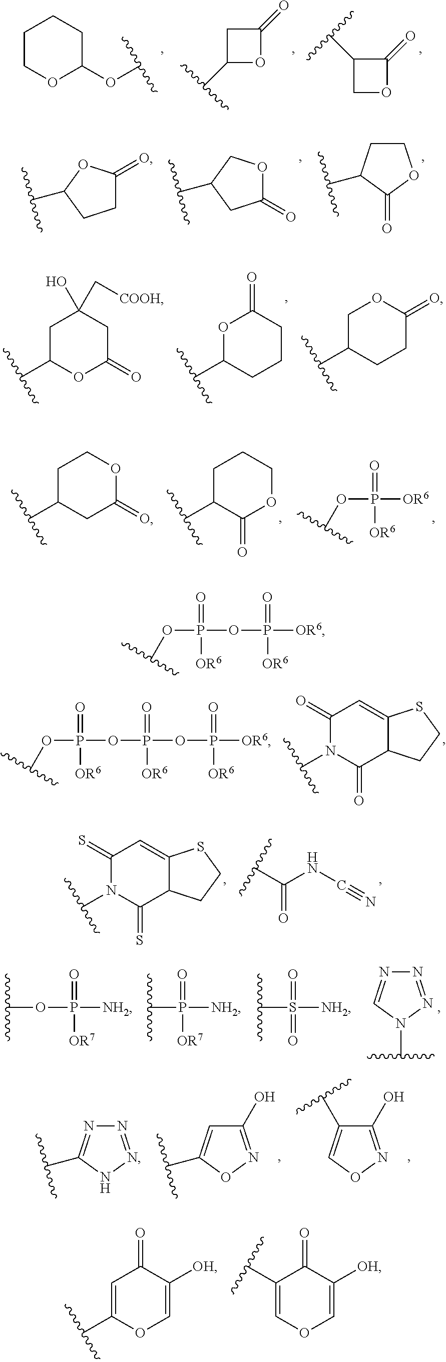

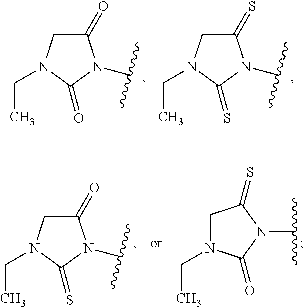

R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

In some embodiments, the compound of Formula (A) is a racemate or a mixture of enantiomers or diastereomers. In some embodiments, the compound of Formula (A) has an olefin isomer configuration of (Z) or (E). In some embodiments, the hydroxyl-bearing allylic carbon atom of the compound of Formula (A) has an (R)- or an (S)-stereochemistry.

In some embodiments, the compound of Formula (A) is a (Z)-isomer (or cis) and has the structure:

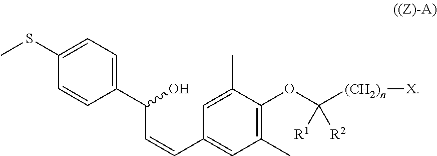

In some embodiments, the compound of Formula ((Z)-A) is substantially free of its corresponding other olefin configuration (i.e., (E)-isomer).

In some embodiments, the compound of formula (A) is an (E)-isomer (or trans) and has the structure:

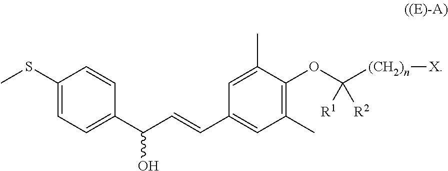

In some embodiments, the compound of Formula ((E)-A) is substantially free of its corresponding other olefin configuration (i.e., (Z)-isomer).

In some embodiments, the compound of Formula (A) has an hydroxyl-bearing allylic carbon atom having (R)-stereochemistry and has the structure:

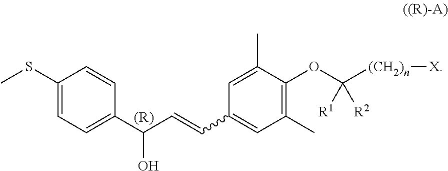

In some embodiments, the compound of Formula ((R)-A) is substantially free of its corresponding opposite stereoisomer, i.e., a compound of Formula (A) whose hydroxyl-bearing allylic carbon atom has an (S)-stereochemistry.

In some embodiments, the hydroxyl-bearing allylic carbon atom of the compound of Formula (A) has an (S)-stereochemistry and has the structure:

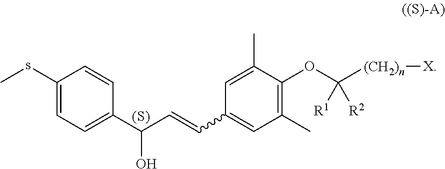

In some embodiments, the compound of Formula ((S)-A) is substantially free of its corresponding opposite stereoisomer, i.e., a compound of Formula (A) whose hydroxyl-bearing allylic carbon atom has an (R)-stereochemistry.

In some embodiments, the compound of Formula (A) is a (Z)-isomer (or cis), has an hydroxyl-bearing allylic carbon atom having an (R)-stereochemistry and has the structure:

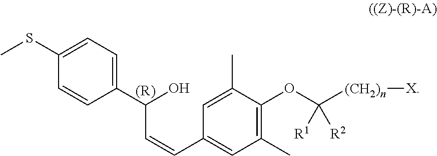

In some embodiments, the compound of Formula ((Z)-(R)-A) is substantially free of compounds of Formulae ((Z)-(S)-A), ((E)-(R)-A), or ((E)-(S)-A).

In some embodiments, the compound of Formula (A) is a (Z)-isomer (or cis), has an hydroxyl-bearing allylic carbon atom having an (S)-stereochemistry and has the structure:

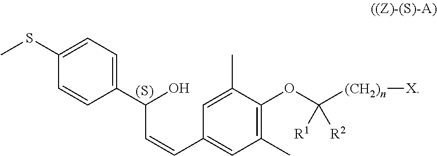

In some embodiments, the compound of Formula ((Z)-(S)-A) is substantially free of compounds of Formulae ((Z)-(R)-A), ((Z)-(R)-A), or ((Z)-(S)-A).

In some embodiments, the compound of Formula (A) is an (E)-isomer (or cis), has an hydroxyl-bearing allylic carbon atom having an (R)-stereochemistry and has the structure:

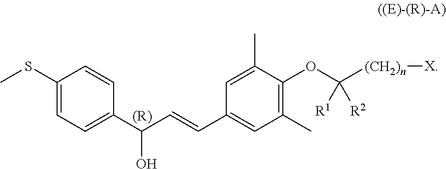

In some embodiments, the compound of Formula ((E)-(R)-A) is substantially free of compounds of Formulae ((E)-(S)-A), ((Z)-(R)-A), or ((Z)-(S)-A).

In some embodiments, the compound of Formula (A) is an (E)-isomer (or cis), has an hydroxyl-bearing allylic carbon atom having an (S)-stereochemistry and has the structure:

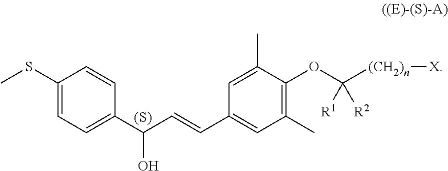

In some embodiments, the compound of Formula ((E)-(S)-A) is substantially free of compounds of Formulae ((E)-(R)-A), ((Z)-(R)-A), or ((Z)-(S)-A).

In some embodiments of compounds of Formula (A), ((Z)-A), ((E)-A), ((R)-A), ((S)-A), ((E)-(R)-A), ((E)-(S)-A), ((Z)-(R)-A), or ((Z)-(S)-A) each R¹ and R² is independently —C₁-C₆ alkyl. In some embodiments, each R¹ and R² is independently —C₁-C₃ alkyl. In some embodiments, each R¹ and R² is independently methyl.

In some embodiments of compounds of formula (A), ((Z)-A), ((E)-A), ((R)-A), ((S)-A), ((E)-(R)-A), ((E)-(S)-A), ((Z)-(R)-A), or ((Z)-(S)-A), X is —CH₂OH, —COOH, —COH, or —COOR³, or —COOCH₂CONR⁴R⁵. In some embodiments, X is —CH₂OH, —COOH, —COOR³, —COOCH₂CONR⁴R⁵. In some embodiments, X is —CH₂OH or —COOH.

In some embodiments of compounds of Formula (A), ((Z)-A), ((E)-A), ((R)-A), ((S)-A), ((E)-(R)-A), ((E)-(S)-A), ((Z)-(R)-A), or ((Z)-(S)-A), R³ is —C₁-C₆ alkyl. In some embodiments, R³ is methyl, ethyl, n-propyl, isopropyl, n-butyl, s-butyl, or t-butyl.

In some embodiments of compounds of Formula (A), ((Z)-A), ((E)-A), ((R)-A), ((S)-A), ((E)-(R)-A), ((E)-(S)-A), ((Z)-(R)-A), or ((Z)-(S)-A), n is 0, 1, 2, or 3. In some embodiments, n is 0, 1, or 2. In some embodiments, n is 0 or 1.

### Compound I

In some embodiments, the compound of the invention is Compound I having the structure:

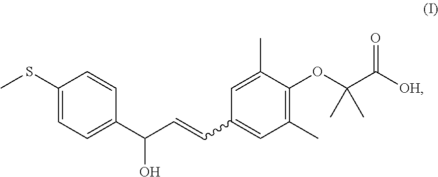 [SMILES: CSc1ccc(C(O)C=Cc2cc(C)c(OC(C)(C)C(=O)O)c(C)c2)cc1]

or a pharmaceutically acceptable salt, solvate, ester, amide, or prodrug thereof.

In some embodiments, Compound I is a racemate or a mixture of enantiomers. In some embodiments, Compound I has an olefin isomer configuration of (Z) or (E). In some embodiments, Compound I has an hydroxyl-bearing allylic carbon atom having an (R)- or an (S)-stereochemistry.

In some embodiments, Compound I is a (Z)-isomer (or cis) and has the structure:

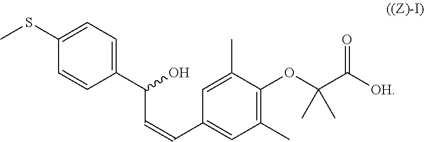 [SMILES: CSc1ccc(C(O)/C=C\c2cc(C)c(OC(C)(C)C(=O)O)c(C)c2)cc1]

In some embodiments, Compound I is a (Z)-isomer and is substantially free of its corresponding other olefin configuration (i.e., (E)-isomer).

In some embodiments, Compound I is an (E)-isomer (or trans) and has the structure:

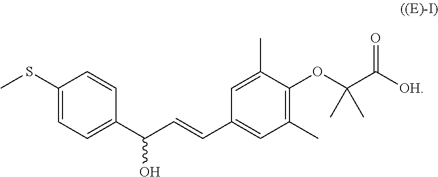 [SMILES: CSc1ccc(C(O)/C=C/c2cc(C)c(OC(C)(C)C(=O)O)c(C)c2)cc1]

In some embodiments, Compound I is an (E)-isomer and is substantially free of its corresponding other olefin configuration (i.e., (Z)-isomer).

In some embodiments, Compound I has an hydroxyl-bearing allylic carbon atom having an (R)-enantiomer and has the structure:

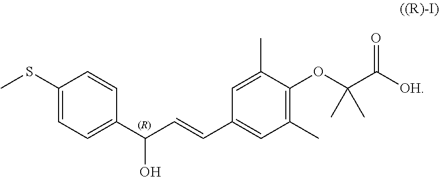

In some embodiments, Compound I has an hydroxyl-bearing allylic carbon atom having an (R)-enantiomer and is substantially free of its corresponding opposite enantiomer (i.e., (S)-enantiomer).

In some embodiments, Compound I has an hydroxyl-bearing allylic carbon atom having an (S)-enantiomer and has the structure:

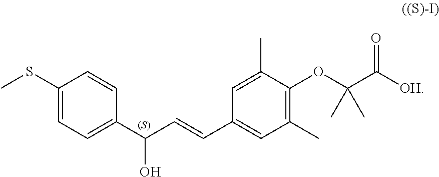

In some embodiments, Compound I has an hydroxyl-bearing allylic carbon atom having an (S)-enantiomer and is substantially free of its corresponding opposite enantiomer (i.e., (R)-enantiomer). In some embodiments, Compound I is a non-racemic mixture of its (R)-enantiomer and (S)-enantiomer. In some embodiments, the non-racemic mixture has an excess of (R)-enantiomer relative to (S)-enantiomer. In some embodiments, the non-racemic mixture has an excess of (S)-enantiomer relative to (R)-enantiomer.

In some embodiments, Compound I is a (Z)-isomer (or cis), has an hydroxyl-bearing allylic carbon atom having an (R)-enantiomer and has the structure:

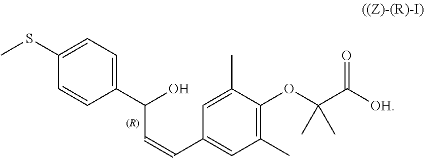

In some embodiments, Compound ((Z)-(R)-I) is substantially free of Compounds ((Z)-(S)-I), ((E)-(R)-I), or ((E)-(S)-I).

In some embodiments, Compound I is a (Z)-isomer, has an hydroxyl-bearing allylic carbon atom having an (S)-enantiomer and has the structure:

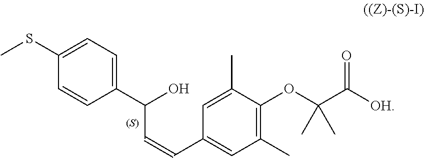

In some embodiments, Compound ((Z)-(S)-I) is substantially free of Compounds ((Z)-(R)-I), ((E)-(R)-I), or ((E)-(S)-I).

In some embodiments, Compound I is an (E)-isomer (or trans), has an hydroxyl-bearing allylic carbon atom having an (R)-enantiomer and has the structure:

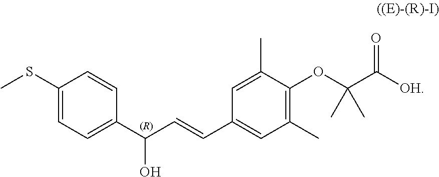

In some embodiments, Compound ((E)-(R)-I) is substantially free of Compounds (E)-(S)-I), ((Z)-(R)-I), or ((Z)-(S)-I).

In some embodiments, Compound I is an (E)-isomer, has an hydroxyl-bearing allylic carbon atom having an (S)-enantiomer and has the structure:

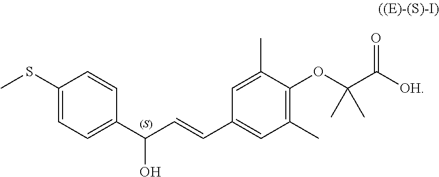

In some embodiments, Compound ((E)-(S)-I) is substantially free of Compounds ((E)-(R)-I), ((Z)-(R)-I), or ((Z)-(S)-I).

### Compound III

In some embodiments, the compound of the invention is Compound III having the structure:

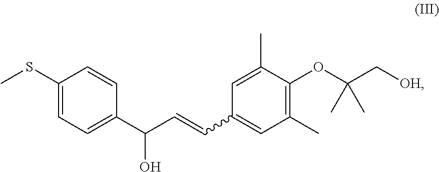 [SMILES: CSc1ccc(C(O)C=Cc2cc(C)c(OC(C)(C)CO)c(C)c2)cc1]

or a pharmaceutically acceptable salt, solvate, ester, amide, or prodrug thereof.

In some embodiments, Compound III is a racemate or a mixture of enantiomers. In some embodiments, Compound III has an olefin isomer configuration of (Z) or (E). In some embodiments, Compound III has an hydroxyl-bearing allylic carbon atom having an (R)- or an (S)-stereochemistry.

In some embodiments, Compound III is a (Z)-isomer (or cis) and has the structure:

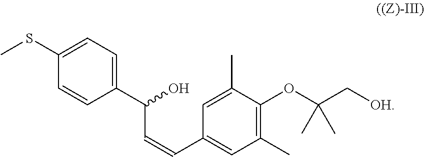 [SMILES: CSc1ccc(C(O)/C=C\c2cc(C)c(OC(C)(C)CO)c(C)c2)cc1]

In some embodiments, Compound III is a (Z)-isomer and is substantially free of its corresponding other olefin configuration (i.e., (E)-isomer).

In some embodiments, Compound III is an (E)-isomer (or trans) and has the structure:

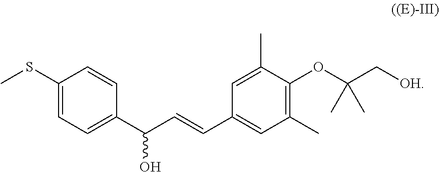 [SMILES: CSc1ccc(C(O)/C=C/c2cc(C)c(OC(C)(C)CO)c(C)c2)cc1]

In some embodiments, Compound III is an (E)-isomer and is substantially free of its corresponding other olefin configuration (i.e., (Z)-isomer).

In some embodiments, Compound III has an hydroxyl-bearing allylic carbon atom having an (R)-enantiomer and has the structure:

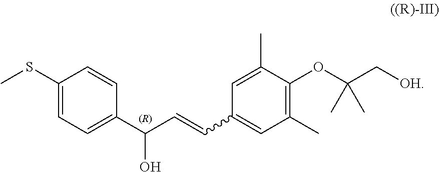

In some embodiments, Compound III has an hydroxyl-bearing allylic carbon atom having an (R)-enantiomer and is substantially free of its corresponding opposite enantiomer (i.e., (S)-enantiomer).

In some embodiments, Compound III has an hydroxyl-bearing allylic carbon atom having an (S)-enantiomer and has the structure:

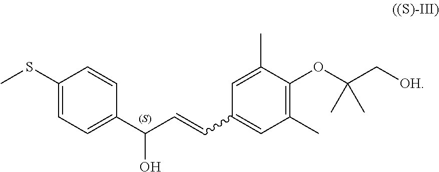

In some embodiments, Compound III has an hydroxyl-bearing allylic carbon atom having an (S)-enantiomer and is substantially free of its corresponding opposite enantiomer (i.e., (R)-enantiomer). In some embodiments, Compound III is a non-racemic mixture of its (R)-enantiomer and (S)-enantiomer. In some embodiments, the non-racemic mixture has an excess of (R)-enantiomer relative to (S)-enantiomer. In some embodiments, the non-racemic mixture has an excess of (S)-enantiomer relative to (R)-enantiomer.

In some embodiments, Compound III is an (Z)-isomer (or cis), has an hydroxyl-bearing allylic carbon atom having an (R)-enantiomer and has the structure:

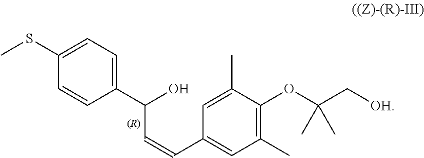

In some embodiments, Compound ((Z)-(R)-III) is substantially free of Compounds ((Z)-(S)-III), ((E)-(R)-III), or ((E)-(S)-III).

In some embodiments, Compound III is an (Z)-isomer, has an hydroxyl-bearing allylic carbon atom having an (S)-enantiomer and has the structure:

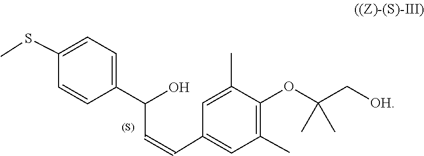

In some embodiments, Compound ((Z)-(S)-III) is substantially free of Compounds ((Z)-(R)-III), ((E)-(R)-III), or ((E)-(S)-III).

In some embodiments, Compound I is an (E)-isomer (or trans), has an hydroxyl-bearing allylic carbon atom having an (R)-enantiomer and has the structure:

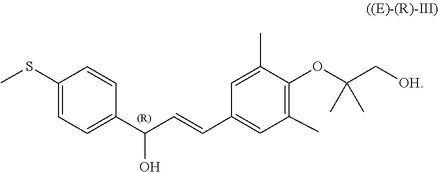

In some embodiments, Compound ((E)-(R)-III) is substantially free of Compounds ((E)-(S)-III), ((Z)-(R)-III), or ((Z)-(S)-III).

In some embodiments, Compound III is an (E)-isomer, has an hydroxyl-bearing allylic carbon atom having an (S)-enantiomer and has the structure:

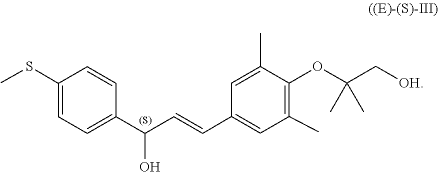

In some embodiments, Compound ((E)-(S)-III) is substantially free of Compounds ((E)-(R)-III), ((Z)-(R)-III), or ((Z)-(S)-III).

### Compounds of Formula (B)

In some embodiments, the compounds of the invention are compounds of Formula (B):

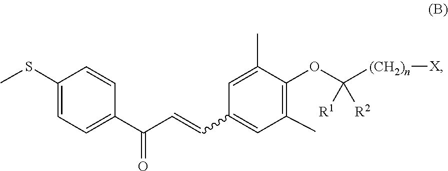

or a pharmaceutically acceptable salt, solvate ester, amide, or prodrug thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COH, —COOCH₂CONR⁴R⁵, —SO₃H,

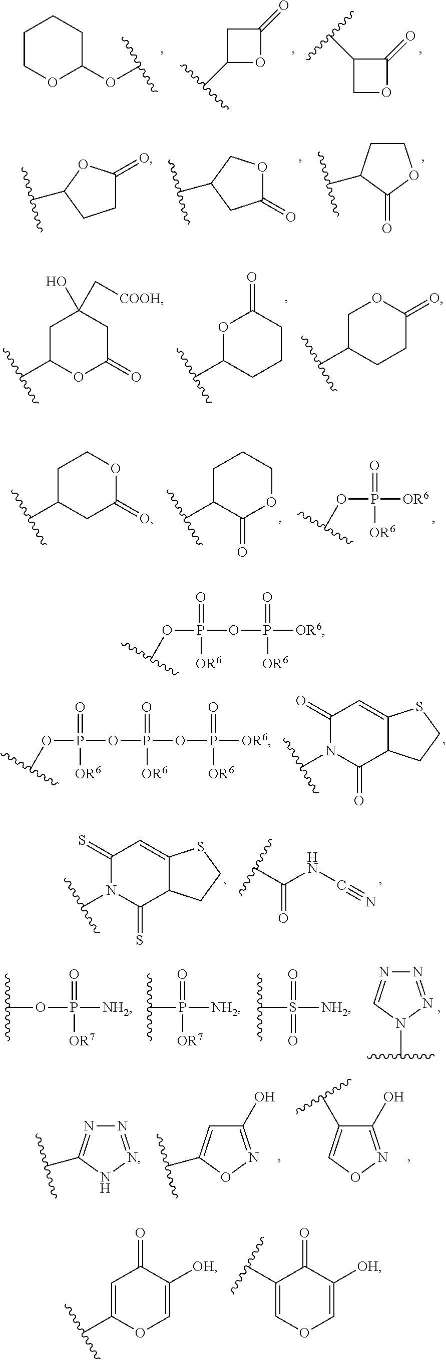

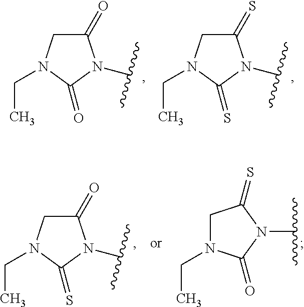

R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

In some embodiments, the compound of Formula (B) is a racemate or a mixture of enantiomers. In some embodiments, the compounds of Formula (B) has an olefin isomer configuration of (Z) or (E).

In some embodiments, the compounds of Formula (B) is a (Z)-isomer (or cis) and has the structure:

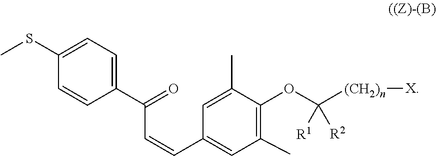

In some embodiments, the compounds of Formula (B) is a (Z)-isomer and is substantially free of its corresponding other olefin configuration (i.e., (E)-isomer).

In some embodiments, the compounds of Formula (B) is an (E)-isomer (or trans) and has the structure:

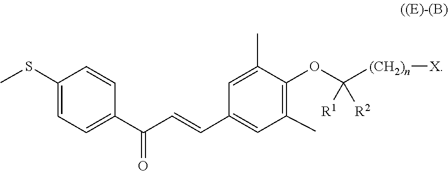

In some embodiments, the compounds of Formula (B) is an (E)-isomer and is substantially free of its corresponding other olefin configuration (i.e., (Z)-isomer).

In some embodiments of compounds of Formula (B), each R¹ and R² is independently —C₁-C₆ alkyl. In some embodiments, each R¹ and R² is independently —C₁-C₃ alkyl. In some embodiments, each R¹ and R² is independently methyl.

In some embodiments of compounds of Formula (B), X is —CH₂OH, —COH, or —COOCH₂CONR⁴R⁵. In some embodiments, X is —CH₂OH.

In some embodiments of compounds of Formula (B), R³ is —C₁-C₆ alkyl. In some embodiments, R³ is methyl, ethyl, n-propyl, isopropyl, n-butyl, s-butyl, or t-butyl.

In some embodiments of compounds of Formula (B), n is 0, 1, 2, or 3. In some embodiments, n is 0, 1, or 2. In some embodiments, n is 0 or 1.

### Compound II

In some embodiments, the compound of the invention is Compound II having the structure:

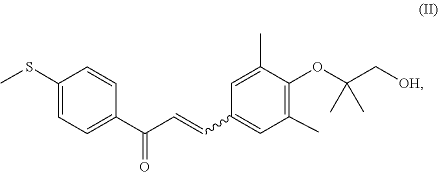 [SMILES: CSc1ccc(C(=O)C=Cc2cc(C)c(OC(C)(C)CO)c(C)c2)cc1]

or a pharmaceutically acceptable salt, solvate, ester, amide, or prodrug thereof.

In some embodiments, Compound II has an olefin isomer configuration of (Z) or (E).

In some embodiments, Compound II is a (Z)-isomer (or cis) and has the structure:

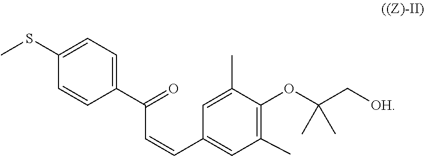 [SMILES: CSc1ccc(C(=O)/C=C\c2cc(C)c(OC(C)(C)CO)c(C)c2)cc1]

In some embodiments, Compound II is a (Z)-isomer and is substantially free of its corresponding other olefin configuration (i.e., (E)-isomer).

In some embodiments, Compound 11 is an (E)-isomer (or trans) and has the structure:

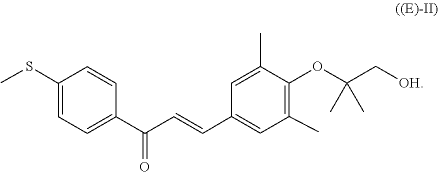 [SMILES: CSc1ccc(C(=O)/C=C/c2cc(C)c(OC(C)(C)CO)c(C)c2)cc1]

In some embodiments, Compound II is an (E)-isomer and is substantially free of its corresponding other olefin configuration (i.e., (Z)-isomer).

### Compositions of the Invention

In some embodiments, the compositions of the invention comprise (i) an effective amount of a compound of the invention and (ii) a pharmaceutically acceptable carrier or vehicle.

In some embodiments, the compositions of the invention comprise (i) an effective amount of Compound I or a pharmaceutically acceptable salt, solvate, ester, amide, or prodrug thereof and (ii) a pharmaceutically acceptable carrier or vehicle. In some embodiments, the compositions of the invention comprise (i) an effective amount of a racemate of Compound I or a pharmaceutically acceptable salt, solvate, ester, amide, or prodrug thereof and (ii) a pharmaceutically acceptable carrier or vehicle. In some embodiments, the compositions of the invention comprise (i) an effective amount of a mixture of enantiomers of Compound I or a pharmaceutically acceptable salt, solvate, ester, amide, or prodrug thereof and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I has an hydroxyl-bearing allylic carbon atom having an (R)-enantiomer, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I has an hydroxyl-bearing allylic carbon atom having an (R)-enantiomer, and (ii) a pharmaceutically acceptable carrier or vehicle, wherein the compositions are substantially free of the (S)-enantiomer of Compound I or a pharmaceutically acceptable salt or thereof. In some embodiments, the compositions of the invention comprise (i) an effective amount of Compound I or a pharmaceutically acceptable salt, solvate, ester, amide, or prodrug thereof, wherein Compound I has an hydroxyl-bearing allylic carbon atom having an (S)-enantiomer, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I has an hydroxyl-bearing allylic carbon atom having an (S)-enantiomer, and (ii) a pharmaceutically acceptable carrier or vehicle, wherein the compositions are substantially free of the (R)-enantiomer of Compound I or a pharmaceutically acceptable salt, solvate, ester, amide, or prodrug thereof. In some embodiments, the compositions of the invention comprise (i) an effective amount of a non-racemic mixture of an (R)-enantiomer and an (S)-enantiomer of Compound I or a pharmaceutically acceptable salt, solvate, ester, amide, or prodrug thereof and (ii) a pharmaceutically acceptable carrier or vehicle. In some embodiments, the non-racemic mixture has an excess of (R)-enantiomer relative to (S)-enantiomer. In some embodiments, the non-racemic mixture has an excess of (S)-enantiomer relative to (R)-enantiomer.

In some embodiments, the compositions of the invention comprise (i) an effective amount of a (Z)-isomer of Compound I or a pharmaceutically acceptable salt, solvate, ester, amide, or prodrug thereof and (ii) a pharmaceutically acceptable carrier or vehicle. In some embodiments, the compositions of the invention comprise (i) an effective amount of a (Z)-isomer of Compound I or a pharmaceutically acceptable salt, solvate, ester, amide, or prodrug thereof and (ii) a pharmaceutically acceptable carrier or vehicle, wherein the compositions are substantially free of the (E)-isomer of Compound I or a pharmaceutically acceptable salt or thereof. In some embodiments, the compositions of the invention comprise (i) an effective amount of an (E)-isomer of Compound I or a pharmaceutically acceptable salt, solvate, ester, amide, or prodrug thereof and (ii) a pharmaceutically acceptable carrier or vehicle. In some embodiments, the compositions of the invention comprise (i) an effective amount of an (E)-isomer of Compound I or a pharmaceutically acceptable salt, solvate, ester, amide, or prodrug thereof and (ii) a pharmaceutically acceptable carrier or vehicle, wherein the compositions are substantially free of the (Z)-isomer of Compound I or a pharmaceutically acceptable salt, solvate, ester, amide, or prodrug thereof. In some embodiments, the compositions of the invention comprise (i) an effective amount of a non-equal mixture of a (Z)-isomer and an (E)-isomer of Compound I or a pharmaceutically acceptable salt, solvate, ester, amide, or prodrug thereof and (ii) a pharmaceutically acceptable carrier or vehicle. In some embodiments, the non-equal mixture has an excess of (Z)-isomer relative to (E)-isomer. In some embodiments, the non-equal mixture has an excess of (E)-isomer relative to (Z)-isomer.

In some embodiments, the compositions of the invention comprise (i) an effective amount of Compound I or a pharmaceutically acceptable salt, solvate, ester, amide, or prodrug thereof, wherein Compound I is an (Z)-isomer and has an hydroxyl-bearing allylic carbon atom having an (R)-stereochemistry, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I is an (Z)-isomer and has an hydroxyl-bearing allylic carbon atom having an (R)-stereochemistry, and (ii) a pharmaceutically acceptable carrier or vehicle, wherein the compositions are substantially free of Compounds ((Z)-(S)-I), ((E)-(R)-I), or ((E)-(S)-I), or a pharmaceutically acceptable salt or thereof. In some embodiments, the compositions of the invention comprise (i) an effective amount of Compound I or a pharmaceutically acceptable salt, solvate, ester, amide, or prodrug thereof, wherein Compound I is an (Z)-isomer and has an hydroxyl-bearing allylic carbon atom having an (S)-stereochemistry, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I is an (Z)-isomer and has an hydroxyl-bearing allylic carbon atom having an (S)-stereochemistry, and (ii) a pharmaceutically acceptable carrier or vehicle, wherein the compositions are substantially free of Compounds ((Z)-(R)-I), ((E)-(R)-I), or ((E)-(S)-I), or a pharmaceutically acceptable salt or thereof. In some embodiments, the compositions of the invention comprise (i) an effective amount of Compound I or a pharmaceutically acceptable salt, solvate, ester, amide, or prodrug thereof, wherein Compound I is an (E)-isomer and has an hydroxyl-bearing allylic carbon atom having an (R)-stereochemistry, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I is an (E)-isomer and has an hydroxyl-bearing allylic carbon atom having an (R)-stereochemistry, (ii) a pharmaceutically acceptable carrier or vehicle, wherein the compositions are substantially free of Compounds (E)-(S)-I), ((Z)-(R)-I), or ((Z)-(S)-I), or a pharmaceutically acceptable salt or thereof. In some embodiments, the compositions of the invention comprise (i) an effective amount of Compound I or a pharmaceutically acceptable salt, solvate, ester, amide, or prodrug thereof, wherein Compound I is an (E)-isomer and has an hydroxyl-bearing allylic carbon atom having an (S)-stereochemistry, and (ii) a pharmaceutically acceptable carrier or vehicle. In some embodiments, the compositions of the invention comprise (i) an effective amount of Compound I or a pharmaceutically acceptable salt, solvate, ester, amide, or prodrug thereof, wherein Compound I is an (E)-isomer and has an hydroxyl-bearing allylic carbon atom having an (S)-stereochemistry, and (ii) a pharmaceutically acceptable carrier or vehicle, wherein the compositions are substantially free of Compounds ((E)-(R)-I), ((Z)-(R)-I), or ((Z)-(S)-I), or a pharmaceutically acceptable salt or thereof.

In some embodiments, the composition of the invention comprises (i) an effective amount of Compound II or a pharmaceutically acceptable salt, solvate, ester, amide, or prodrug thereof and (ii) a pharmaceutically acceptable carrier or vehicle. In some embodiments, the composition of the invention comprises (i) an effective amount of a (Z)-isomer of Compound II or a pharmaceutically acceptable salt, solvate, ester, amide, or prodrug thereof and (ii) a pharmaceutically acceptable carrier or vehicle. In some embodiments, the composition of the invention comprises (i) an effective amount of a (Z)-isomer of Compound II or a pharmaceutically acceptable salt, solvate, ester, amide, or prodrug thereof and (ii) a pharmaceutically acceptable carrier or vehicle, wherein the compositions are substantially free of the (E)-isomer of Compound II or a pharmaceutically acceptable salt or thereof. In some embodiments, the composition of the invention comprises (i) an effective amount of an (E)-isomer of Compound II or a pharmaceutically acceptable salt, solvate, ester, amide, or prodrug thereof and (ii) a pharmaceutically acceptable carrier or vehicle. In some embodiments, the composition of the invention comprises (i) an effective amount of an (E)-isomer of Compound II or a pharmaceutically acceptable salt, solvate, ester, amide, or prodrug thereof and (ii) a pharmaceutically acceptable carrier or vehicle, wherein the compositions are substantially free of the (Z)-isomer of Compound II or a pharmaceutically acceptable salt or thereof. In some embodiments, the compositions of the invention comprise (i) an effective amount of a non-equal mixture of a (Z)-isomer and an (E)-isomer of Compound II or a pharmaceutically acceptable salt, solvate, ester, amide, or prodrug thereof and (ii) a pharmaceutically acceptable carrier or vehicle. In some embodiments, the non-equal mixture has an excess of (Z)-isomer relative to (E)-isomer. In some embodiments, the non-equal mixture has an excess of (E)-isomer relative to (Z)-isomer.

In some embodiments, the compositions of the invention comprise (i) an effective amount of Compound III or a pharmaceutically acceptable salt, solvate, ester, amide, or prodrug thereof and (ii) a pharmaceutically acceptable carrier or vehicle. In some embodiments, the compositions of the invention comprise (i) an effective amount of a racemate of Compound III or a pharmaceutically acceptable salt, solvate, ester, amide, or prodrug thereof and (ii) a pharmaceutically acceptable carrier or vehicle. In some embodiments, the compositions of the invention comprise (i) an effective amount of a mixture of enantiomers of Compound III or a pharmaceutically acceptable salt, solvate, ester, amide, or prodrug thereof and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III has an hydroxyl-bearing allylic carbon atom having an (R)-enantiomer, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III has an hydroxyl-bearing allylic carbon atom having an (R)-enantiomer, and (ii) a pharmaceutically acceptable carrier or vehicle, wherein the compositions are substantially free of the (S)-enantiomer of Compound III or a pharmaceutically acceptable salt or thereof. In some embodiments, the compositions of the invention comprise (i) an effective amount of Compound III or a pharmaceutically acceptable salt, solvate, ester, amide, or prodrug thereof, wherein Compound III has an hydroxyl-bearing allylic carbon atom having an (S)-enantiomer, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III has an hydroxyl-bearing allylic carbon atom having an (S)-enantiomer, and (ii) a pharmaceutically acceptable carrier or vehicle, wherein the compositions are substantially free of the (R)-enantiomer of Compound III or a pharmaceutically acceptable salt, solvate, ester, amide, or prodrug thereof. In some embodiments, the compositions of the invention comprise (i) an effective amount of a non-racemic mixture of an (R)-enantiomer and an (S)-enantiomer of Compound III or a pharmaceutically acceptable salt, solvate, ester, amide, or prodrug thereof and (ii) a pharmaceutically acceptable carrier or vehicle. In some embodiments, the non-racemic mixture has an excess of (R)-enantiomer relative to (S)-enantiomer. In some embodiments, the non-racemic mixture has an excess of (S)-enantiomer relative to (R)-enantiomer.

In some embodiments, the compositions of the invention comprise (i) an effective amount of a (Z)-isomer of Compound III or a pharmaceutically acceptable salt, solvate, ester, amide, or prodrug thereof and (ii) a pharmaceutically acceptable carrier or vehicle. In some embodiments, the compositions of the invention comprise (i) an effective amount of a (Z)-isomer of Compound III or a pharmaceutically acceptable salt, solvate, ester, amide, or prodrug thereof and (ii) a pharmaceutically acceptable carrier or vehicle, wherein the compositions are substantially free of the (E)-isomer of Compound III or a pharmaceutically acceptable salt or thereof. In some embodiments, the compositions of the invention comprise (i) an effective amount of an (E)-isomer of Compound III or a pharmaceutically acceptable salt, solvate, ester, amide, or prodrug thereof and (ii) a pharmaceutically acceptable carrier or vehicle. In some embodiments, the compositions of the invention comprise (i) an effective amount of an (E)-isomer of Compound III or a pharmaceutically acceptable salt, solvate, ester, amide, or prodrug thereof and (ii) a pharmaceutically acceptable carrier or vehicle, wherein the compositions are substantially free of the (Z)-isomer of Compound III or a pharmaceutically acceptable salt, solvate, ester, amide, or prodrug thereof. In some embodiments, the compositions of the invention comprise (i) an effective amount of a non-equal mixture of a (Z)-isomer and an (E)-isomer of Compound III or a pharmaceutically acceptable salt, solvate, ester, amide, or prodrug thereof and (ii) a pharmaceutically acceptable carrier or vehicle. In some embodiments, the non-equal mixture has an excess of (Z)-isomer relative to (E)-isomer. In some embodiments, the non-equal mixture has an excess of (E)-isomer relative to (Z)-isomer.

In some embodiments, the compositions of the invention comprise (i) an effective amount of Compound III or a pharmaceutically acceptable salt, solvate, ester, amide, or prodrug thereof, wherein Compound III is an (Z)-isomer and has an hydroxyl-bearing allylic carbon atom having an (R)-stereochemistry, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III is an (Z)-isomer and has an hydroxyl-bearing allylic carbon atom having an (R)-stereochemistry, and (ii) a pharmaceutically acceptable carrier or vehicle, wherein the compositions are substantially free of Compounds ((Z)-(S)-III), ((E)-(R)-III), or ((E)-(S)-III), or a pharmaceutically acceptable salt or thereof. In some embodiments, the compositions of the invention comprise (i) an effective amount of Compound III or a pharmaceutically acceptable salt, solvate, ester, amide, or prodrug thereof, wherein Compound III is an (Z)-isomer and has an hydroxyl-bearing allylic carbon atom having an (S)-stereochemistry, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III is an (Z)-isomer and has an hydroxyl-bearing allylic carbon atom having an (S)-stereochemistry, and (ii) a pharmaceutically acceptable carrier or vehicle, wherein the compositions are substantially free of Compounds ((Z)-(R)-III), ((E)-(R)-III), or ((E)-(S)-III), or a pharmaceutically acceptable salt or thereof. In some embodiments, the compositions of the invention comprise (i) an effective amount of Compound III or a pharmaceutically acceptable salt, solvate, ester, amide, or prodrug thereof, wherein Compound III is an (E)-isomer and has an hydroxyl-bearing allylic carbon atom having an (R)-stereochemistry, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III is an (E)-isomer and has an hydroxyl-bearing allylic carbon atom having an (R)-stereochemistry, (ii) a pharmaceutically acceptable carrier or vehicle, wherein the compositions are substantially free of Compounds (E)-(S)-III), ((Z)-(R)-III), or ((Z)-(S)-III), or a pharmaceutically acceptable salt or thereof. In some embodiments, the compositions of the invention comprise (i) an effective amount of Compound III or a pharmaceutically acceptable salt, solvate, ester, amide, or prodrug thereof, wherein Compound III is an (E)-isomer and has an hydroxyl-bearing allylic carbon atom having an (S)-stereochemistry, and (ii) a pharmaceutically acceptable carrier or vehicle. In some embodiments, the compositions of the invention comprise (i) an effective amount of Compound III or a pharmaceutically acceptable salt, solvate, ester, amide, or prodrug thereof, wherein Compound III is an (E)-isomer and has an hydroxyl-bearing allylic carbon atom having an (S)-stereochemistry, and (ii) a pharmaceutically acceptable carrier or vehicle, wherein the compositions are substantially free of Compounds ((E)-(R)-III), ((Z)-(R)-III), or ((Z)-(S)-III), or a pharmaceutically acceptable salt or thereof.

In some embodiments, the compositions of the invention further comprise another therapeutically active agent.

A composition of the invention further comprising another therapeutically active agent is a “combination of the invention.” The present invention provides combinations of the invention. The combination of the invention can comprise one or more therapeutically active agents.

In some embodiments, the other therapeutically active agent is a lipid lowering drug, a statin, a cholesterol absorption inhibitor, an antibody against PCSK9, an siRNA PCSK9, an anti-fibrotic agent, a thyroid hormone, a selective thyroid receptor-β agonist, apoptosis signal-regulating kinase 1 (ASK1) inhibitor, acetyl-CoA carboxylase (ACC) inhibitor, an integrin antagonist, or a non-steroidal Farnesoid X receptor (FXR) agonist.

In some embodiments, the lipid lowering drug is gemfibrozil, fenofibrate, bezafibrate, clofibrate, ciprofibrate, clinofibrate, etofylline, pirifibrate, simfibrate, tocofibrate, or pemafibrate. In some embodiments, the lipid lowering drug is gemfibrozil, fenofibrate, bezafibrate, or pemafibrate.

In some embodiments, statin is atorvastatin, simvastatin, pravastatin, rosuvastatin, fluvastatin, lovastatin, pitavastatin, mevastatin, dalvastatin, dihydrocompactin, or cerivastatin, or a pharmaceutically acceptable salt or solvate thereof. In some embodiments, statin is atorvastatin, simvastatin, pravastatin or rosuvastatin, or a pharmaceutically acceptable salt or solvate thereof.

In some embodiments, the cholesterol absorption inhibitor is ezetimibe.

In some embodiments, the antibody against PCSK9 is evolocumab alirocumab, bococizumab, 1D05-IgG2 (Merck), RG7652 (Genentech), LY3015014 (Eli Lilly), or LGT-209 (Novartis/Cyon Therapeutics). In some embodiments, the antibody against PCSK9 is evolocumab or alirocumab.

In some embodiments, the siRNA PCSK9 is an siRNA interfering with production of PCSK9 such as inclisiran.

In some embodiments, the anti-fibrotic agent is nitazoxamide, tizoxanide, or tizoxanide glucuronide, nintedanib, imatinib, or a pharmaceutically acceptable salt or solvate thereof.

In some embodiments, the selective thyroid receptor-β agonist is VK2809 (Viking Therapeutics), MGL-3196 (Madrigal Pharmaceuticals), MGL-3745 (Madrigal Pharmaceuticals), SKL-14763, sobetirome, BCT304 (ITL Pharma), ZYT1 (Zydus Cadila), MB-0781 (Metabasis), or eprotirome.

In some embodiments, the ASK1 inhibitor is selonsertib.

In some embodiments, the ACC inhibitor is firsocostat.

In some embodiments, the integrin antagonist is an α5β1 inhibitor or a pan integrin inhibitor. In some embodiments, the integrin antagonist is IDL-2965 (Indalo Therapeutics). In some embodiments, the integrin antagonist is CLT-28643 (ClanoTech AB).

In some embodiments, the integrin antagonist is 3-(6-Methoxypyridin-3-yl)-3-(4-(3-(5,6,7,8-tetrahydro-1,8-naphthyridin-2-yl)propyl)-1H-indazol-1-yl)propanoic acid; 3-(6-Methoxypyridin-3-yl)-3-(5-(2-(5,6,7,8-tetrahydro-1,8-naphthyridin-2-yl)ethyl)-1H-indazol-1-yl)propanoic acid; 3-(6-Methoxypyridin-3-yl)-3-(4-(2-(5,6,7,8-tetrahydro-1,8-naphthyridin-2-yl)ethoxy)-1H-indazol-1-yl)propanoic acid; (S)-2-(((Benzyloxy)carbonyl)amino)-3-(5-(2-(5,6,7,8-tetrahydro-1,8-naphth-yridin-2-yl)ethoxy)-2H-indazol-2-yl)propanoic acid; 3-Phenyl-3-(5-(3-(5,6,7,8-tetrahydro-1,8-naphthyridin-2-yl)propoxy)-1H-in-dazol-1-yl)propanoic acid; (S)-3-(6-Methoxypyridin-3-yl)-3-(5-(2-(5,6,7,8-tetrahydro-1,8-naphthyridi-n-2-yl)ethoxy)-2H-indazol-2-yl)propanoic acid; (S)-3-(6-Methoxypyridin-3-yl)-3-(6-(2-(5,6,7,8-tetrahydro-1,8-naphthyridi-n-2-yl)ethyl)-2H-indazol-2-yl)propanoic acid; (S)-3-(6-Methoxypyridin-3-yl)-3-(6-((2-methyl-5,6,7,8-tetrahydro-1,8-naph-thyridin-3-yl)methyl)-2H-indazol-2-yl)propanoic acid; (S)-3-(6-Methoxypyridin-3-yl)-3-(6-(3-(5,6,7,8-tetrahydro-1,8-naphthyridi-n-2-yl)propyl)-2H-indazol-2-yl)propanoic acid; (S)-3-(6-Methoxypyridin-3-yl)-3-(6-(2-(2-methyl-5,6,7,8-tetrahydro-1,8-na-phthyridin-3-yl)ethyl)-2H-indazol-2-yl)propanoic acid; 3-(6-Methoxypyridin-3-yl)-3-(5-(2-(5,6,7,8-tetrahydro-1,8-naphthyridin-2-yl)ethoxy)pyrazolo[4,3-b]pyridin-1-yl)propanoic acid; 3-(5-(2-((4,5-Dihydroimidazol-2-yl)amino)ethoxy)-1H-indazol-1-yl)-3-(6-me-thoxypyridin-3-yl)propanoic acid; 3-(5-(2-(5,6,7,8-Tetrahydro-1,8-naphthyridin-2-yl)ethoxy)-1H-indazol-1-yl-)-2-((2,4,6-trimethylphenyl)sulfonamido)propanoic acid; 2-(((Benzyloxy)carbonyl)amino)-3-(5-(2-(5,6,7,8-tetrahydro-1,8-naphthyrid-in-2-yl)ethoxy)-1H-indazol-1-yl)propanoic acid; 3-(Quinoxalin-2-yl)-3-(5-(2-(5,6,7,8-tetrahydro-1,8-naphthyridin-2-yl)eth-oxy)-1H-indazol-1-yl)propanoic acid; (R)-3-(Quinoxalin-2-yl)-3-(5-(2-(5,6,7,8-tetrahydro-1,8-naphthyridin-2-yl-)ethoxy)-1H-indazol-1-yl)propanoic acid; (S)-3-(Quinoxalin-2-yl)-3-(5-(2-(5,6,7,8-tetrahydro-1,8-naphthyridin-2-yl-)ethoxy)-1H-indazol-1-yl)propanoic acid; 3-(3,5-Dichlorophenyl)-3-(4-(3-(5,6,7,8-tetrahydro-1,8-naphthyridin-2-yl)-propyl)-1H-indazol-1-yl)propanoic acid; 3-(Quinoxalin-2-yl)-3-(4-(3-(5,6,7,8-tetrahydro-1,8-naphthyridin-2-yl)pro-pyl)-1H-indazol-1-yl)propanoic acid; 3-(6-Methoxypyridin-3-yl)-3-(5-(3-(5,6,7,8-tetrahydro-1,8-naphthyridin-2-yl)propyl)-1H-indazol-1-yl)propanoic acid; 3-(3,5-Dichlorophenyl)-3-(5-(3-(5,6,7,8-tetrahydro-1,8-naphthyridin-2-yl)-propyl)-1H-indazol-1-yl)propanoic acid; 3-(Quinoxalin-2-yl)-3-(5-(3-(5,6,7,8-tetrahydro-1,8-naphthyridin-2-yl) pro-pyl)-1H-indazol-1-yl)propanoic acid; 3-(3-(Dimethylcarbamoyl)phenyl)-3-(5-(3-(5,6,7,8-tetrahydro-1,8-naphthyri-din-2-yl)propyl)-1H-indazol-1-yl)propanoic acid; 3-(3-(Dimethylcarbamoyl)phenyl)-3-(5-(2-(5,6,7,8-tetrahydro-1,8-naphthyri-din-2-yl)ethyl)-1H-indazol-1-yl)propanoic acid; 3-(Dibenzo[b,d]furan-3-yl)-3-(5-(2-(5,6,7,8-tetrahydro-1,8-naphthyridin-2- -yl)ethyl)-1H-indazol-1-yl)propanoic acid; 3-(3-((Dimethylamino)methyl)phenyl)-3-(5-(2-(5,6,7,8-tetrahydro-1,8-napht-hyridin-2-yl)ethyl)-1H-indazol-1-yl)propanoic acid; 3-(Quinoxalin-2-yl)-3-(5-(2-(5,6,7,8-tetrahydro-1,8-naphthyridin-2-yl)eth-yl)-1H-indazol-1-yl)propanoic acid; 3-(3,5-Dichlorophenyl)-3-(5-(2-(5,6,7,8-tetrahydro-1,8-naphthyridin-2-yl)-ethyl)-1H-indazol-1-yl)propanoic acid; 3-(3-(Dimethylcarbamoyl)phenyl)-3-(4-(2-(5,6,7,8-tetrahydro-1,8-naphthyri-din-2-yl)ethoxy)-1H-indazol-1-yl)propanoic acid; 3-(Dibenzo[b,d]furan-3-yl)-3-(4-(2-(5,6,7,8-tetrahydro-1,8-naphthyridin-2- -yl)ethoxy)-1H-indazol-1-yl)propanoic acid; 6,6,6-Trifluoro-3-(4-(3-(5,6,7,8-tetrahydro-1,8-naphthyridin-2-yl)propyl)-1H-indazol-1-yl)hexanoic acid; 3-(3,5-Dichlorophenyl)-3-(4-(2-(5,6,7,8-tetrahydro-1,8-naphthyridin-2-yl)-ethoxy)-1H-indazol-1-yl)propanoic acid; 3-(Quinoxalin-2-yl)-3-(4-(2-(5,6,7,8-tetrahydro-1,8-naphthyridin-2-yl)eth-oxy)-1H-indazol-1-yl)propanoic acid; 3-(6-Methoxypyridin-3-yl)-3-(5-(2-(5,6,7,8-tetrahydro-1,8-naphthyridin-2-yl)ethoxy)-1H-indazol-1-yl)propanoic acid; 3-(3-(Dimethylcarbamoyl)phenyl)-3-(5-(2-(5,6,7,8-tetrahydro-1,8-naphthyri-din-2-yl)ethoxy)-1H-indazol-1-yl)propanoic acid; (S)-2-(((Benzyloxy)carbonyl)amino)-3-(5-(3-(5,6,7,8-tetrahydro-1,8-naphth-yridin-2-yl)propyl)-1H-indazol-1-yl)propanoic acid; (R)-3-(3-(Dimethylcarbamoyl)phenyl)-3-(5-(2-(5,6,7,8-tetrahydro-1,8-napht-hyridin-2-yl)ethoxy)-1H-indazol-1-yl)propanoic acid; (S)-3-(3-(Dimethylcarbamoyl)phenyl)-3-(5-(2-(5,6,7,8-tetrahydro-1,8-napht-hyridin-2-yl)ethoxy)-1H-indazol-1-yl)propanoic acid; 2-(((Benzyloxy)carbonyl)amino)-3-(4-(2-(5,6,7,8-tetrahydro-1,8-naphthyrid-in-2-yl)ethoxy)-1H-indazol-1-yl)propanoic acid; (R)-3-(6-Methoxypyridin-3-yl)-3-(5-(2-(5,6,7,8-tetrahydro-1,8-naphthyridi-n-2-yl)ethoxy)-1H-indazol-1-yl)propanoic acid; (S)-3-(6-Methoxypyridin-3-yl)-3-(5-(2-(5,6,7,8-tetrahydro-1,8-naphthyridi-n-2-yl)ethoxy)-1H-indazol-1-yl)propanoic acid; 3-(5-Chloropyridin-3-yl)-3-(5-(2-(5,6,7,8-tetrahydro-1,8-naphthyridin-2-y-l)ethoxy)-1H-indazol-1-yl)propanoic acid; 3-(3,5-Dichlorophenyl)-3-(5-(2-(5,6,7,8-tetrahydro-1,8-naphthyridin-2-yl)-ethoxy)-1H-indazol-1-yl)propanoic acid; 3-(3-Fluoro-4-methoxyphenyl)-3-(5-(2-(5,6,7,8-tetrahydro-1,8-naphthyridin-2-yl)ethoxy)-1H-indazol-1-yl)propanoic acid; 3-Cyclopropyl-3-(5-(2-(5,6,7,8-tetrahydro-1,8-naphthyridin-2-yl)ethoxy)-1-H-indazol-1-yl)propanoic acid; 3-(5-(2-(5,6,7,8-Tetrahydro-1,8-naphthyridin-2-yl)ethoxy)-1H-indazol-1-yl-)octanoic acid; 3-(2,3-Dihydrobenzofuran-5-yl)-3-(5-(2-(5,6,7,8-tetrahydro-1,8-naphthyrid-in-2-yl)ethoxy)-1H-indazol-1-yl)propanoic acid; 3-(Quinolin-3-yl)-3-(5-(2-(5,6,7,8-tetrahydro-1,8-naphthyridin-2-yl)ethox-y)-1H-indazol-1-yl)propanoic acid (48); 3-(5-(2-(5,6,7,8-Tetrahydro-1,8-naphthyridin-2-yl)ethoxy)-1H-indazol-1-yl-)-3-(thiophen-2-yl)propanoic acid; 3-(Pyridin-3-yl)-3-(5-(2-(5,6,7,8-tetrahydro-1,8-naphthyridin-2-yl)ethoxy-)-1H-indazol-1-yl)propanoic acid; 3-(5-(2-(5,6,7,8-Tetrahydro-1,8-naphthyridin-2-yl)ethoxy)-1H-indazol-1-yl-)propanoic acid; 3-(3-Cyanophenyl)-3-(5-(2-(5,6,7,8-tetrahydro-1,8-naphthyridin-2-yl)ethox-y)-1H-indazol-1-yl)propanoic acid; 3-(5-Fluoropyridin-2-yl)-3-(5-(2-(5,6,7,8-tetrahydro-1,8-naphthyridin-2-y-l)ethoxy)-1H-indazol-1-yl)propanoic acid; 3-(Dibenzo[b,d]furan-2-yl)-3-(5-(2-(5,6,7,8-tetrahydro-1,8-naphthyridin-2- -yl)ethoxy)-1H-indazol-1-yl)propanoic acid; 3-(4,6-Dimethylpyrimidin-2-yl)-3-(5-(2-(5,6,7,8-tetrahydro-1,8-naphthyrid-in-2-yl)ethoxy)-1H-indazol-1-yl)propanoic acid; 3-(2-Methylbenzo[d]thiazol-5-yl)-3-(5-(2-(5,6,7,8-tetrahydro-1,8-naphthyridin-2-yl)ethoxy)-1H-indazol-1-yl)propanoic acid; 3-(4-Phenoxyphenyl)-3-(5-(2-(5,6,7,8-tetrahydro-1,8-naphthyridin-2-yl)eth-oxy)-1H-indazol-1-yl)propanoic acid; 3-(3-Morpholinophenyl)-3-(5-(2-(5,6,7,8-tetrahydro-1,8-naphthyridin-2-yl)-ethoxy)-1H-indazol-1-yl)propanoic acid; 3-(3-(1H-Pyrrol-1-yl)phenyl)-3-(5-(2-(5,6,7,8-tetrahydro-1,8-naphthyridin-2-yl)ethoxy)-1H-indazol-1-yl)propanoic acid; 3-(3-((Dimethylamino)methyl)phenyl)-3-(5-(2-(5,6,7,8-tetrahydro-1,8-napht-hyridin-2-yl)ethoxy)-1H-indazol-1-yl)propanoic acid; 3-(Pyridin-2-yl)-3-(5-(2-(5,6,7,8-tetrahydro-1,8-naphthyridin-2-yl)ethoxy-)-1H-indazol-1-yl)propanoic acid; 3-(3-(2-Oxopyrrolidin-1-yl)phenyl)-3-(5-(2-(5,6,7,8-tetrahydro-1,8-naphth-yridin-2-yl)ethoxy)-1H-indazol-1-yl)propanoic acid; 3-(1-Propylpyrazol-4-yl)-3-(5-(2-(5,6,7,8-tetrahydro-1,8-naphthyridin-2-y-l)ethoxy)-1H-indazol-1-yl)propanoic acid; (S)-3-(6-Methoxypyridin-3-yl)-3-(5-(2-(5,6,7,8-tetrahydro-1,8-naphthyridi-n-2-yl)ethyl)-2H-indazol-2-yl)propanoic acid; (S)-3-(6-Methoxypyridin-3-yl)-3-(6-(2-(5,6,7,8-tetrahydro-1,8-naphthyridi-n-2-yl)ethoxy)-2H-indazol-2-yl)propanoic acid; 3-(2-Methylpyrimidin-5-yl)-3-(5-(2-(5,6,7,8-tetrahydro-1,8-naphthyridin-2- -yl)ethoxy)-1H-indazol-1-yl)propanoic acid; (S)-3-(6-Methoxypyridin-3-yl)-3-(5-(3-(5,6,7,8-tetrahydro-1,8-naphthyridi-n-2-yl)propyl)-2H-indazol-2-yl)propanoic acid; 3-(3-(3,5-Dimethylpyrazol-1-yl)phenyl)-3-(5-(2-(5,6,7,8-tetrahydro-1,8-na-phthyridin-2-yl)ethoxy)-1H-indazol-1-yl)propanoic acid; 4-(4-(Benzyloxy)phenyl)-3-(5-(2-(5,6,7,8-tetrahydro-1,8-naphthyridin-2-yl-)ethoxy)-1H-indazol-1-yl)butanoic acid; 3-(6-Methoxypyridin-3-yl)-3-(3-methyl-5-(2-(5,6,7,8-tetrahydro-1,8-naphth-yridin-2-yl)ethoxy)-1H-indazol-1-yl)propanoic acid; 3-(1-Methyl-2-oxo-1,2-dihydropyridin-4-yl)-3-(5-(2-(5,6,7,8-tetrahydro-1,-8-naphthyridin-2-yl)ethoxy)-1H-indazol-1-yl)propanoic acid; (3S)-3-(6-Methoxypyridin-3-yl)-3-(6-(2-(1,2,3,4-tetrahydro-1,8-naphthyrid-in-2-yl)ethyl)-2H-indazol-2-yl)propanoic acid; 4-Phenyl-2-((5-(2-(5,6,7,8-tetrahydro-1,8-naphthyridin-2-yl)ethoxy)-1H-in-dazol-1-yl)methyl)butanoic acid; 3-(1-(tert-Butoxycarbonyl)piperidin-4-yl)-3-(5-(2-(5,6,7,8-tetrahydro-1,8- -naphthyridin-2-yl)ethoxy)-1H-indazol-1-yl)propanoic acid; 3-(Pyridin-4-yl)-3-(5-(2-(5,6,7,8-tetrahydro-1,8-naphthyridin-2-yl)ethoxy-)-1H-indazol-1-yl)propanoic acid; 2-(1-(5-(2-(5,6,7,8-Tetrahydro-1,8-naphthyridin-2-yl)ethoxy)-1H-indazol-1- -yl)cyclopropyl)acetic acid; 3-(2-Ethoxypyrimidin-5-yl)-3-(5-(2-(5,6,7,8-tetrahydro-1,8-naphthyridin-2- -yl)ethoxy)-1H-indazol-1-yl)propanoic acid; 4-(4-Fluorophenyl)-3-(5-(2-(5,6,7,8-tetrahydro-1,8-naphthyridin-2-yl)etho-xy)-1H-indazol-1-yl)butanoic acid; 3-(5-Methoxypyrazin-2-yl)-3-(5-(2-(5,6,7,8-tetrahydro-1,8-naphthyridin-2-yl)ethoxy)-1H-indazol-1-yl)propanoic acid; 3-(Quinoxalin-6-yl)-3-(5-(2-(5,6,7,8-tetrahydro-1,8-naphthyridin-2-yl)eth-oxy)-1H-indazol-1-yl)propanoic acid; (S)-3-(Quinolin-3-yl)-3-(6-(2-(5,6,7,8-tetrahydro-1,8-naphthyridin-2-yl)e-thyl)-2H-indazol-2-yl)propanoic acid; (S)-3-(6-((2-Methyl-5,6,7,8-tetrahydro-1,8-naphthyridin-3-yl)methyl)-2H-i-ndazol-2-yl)-3-(quinolin-3-yl)propanoic acid; (3S)-3-(Quinolin-3-yl)-3-(6-(2-(1,2,3,4-tetrahydro-1,8-naphthyridin-2-yl)-ethyl)-2H-indazol-2-yl)propanoic acid; (S)-3-(3-(2-Oxopyrrolidin-1-yl)phenyl)-3-(5-(2-(5,6,7,8-tetrahydro-1,8-na-phthyridin-2-yl)ethoxy)-1H-indazol-1-yl)propanoic acid; (R)-3-(3-(2-Oxopyrrolidin-1-yl)phenyl)-3-(5-(2-(5,6,7,8-tetrahydro-1,8-na-phthyridin-2-yl)ethoxy)-1H-indazol-1-yl)propanoic acid; 3-(5-Fluoro-6-methoxypyridin-3-yl)-3-(5-(2-(5,6,7,8-tetrahydro-1,8-naphth-yridin-2-yl)ethoxy)-1H-indazol-1-yl)propanoic acid; (S)-3-(Quinolin-3-yl)-3-(6-(3-(5,6,7,8-tetrahydro-1,8-naphthyridin-2-yl)p-ropyl)-2H-indazol-2-yl)propanoic acid; (3S)-3-(Quinolin-3-yl)-3-(6-(3-(1,2,3,4-tetrahydro-1,8-naphthyridin-2-yl)-propyl)-2H-indazol-2-yl)propanoic acid; 3-(5-(Hydroxymethyl)pyridin-3-yl)-3-(5-(2-(5,6,7,8-tetrahydro-1,8-naphthy-ridin-2-yl)ethoxy)-1H-indazol-1-yl)propanoic acid; 3-(6-(2-Hydroxy-2-methylpropoxy)-5-methylpyridin-3-yl)-3-(5-(2-(5,6,7,8-t-etrahydro-1,8-naphthyridin-2-yl)ethoxy)-1H-indazol-1-yl)propanoic acid; 3-(3,4-Dihydro-2H-pyrido[3,2-b][1,4]oxazin-6-yl)-3-(5-(2-(5,6,7,8-tetrahy-dro-1,8-naphthyridin-2-yl)ethoxy)-1H-indazol-1-yl)propanoic acid; 3-(2-Methoxypyrimidin-5-yl)-3-(5-(2-(5,6,7,8-tetrahydro-1,8-naphthyridin-2-yl)ethoxy)-1H-indazol-1-yl)propanoic acid; 3-(2-(2-Oxopyrrolidin-1-yl)pyridin-4-yl)-3-(5-(2-(5,6,7,8-tetrahydro-1,8-naphthyridin-2-yl)ethoxy)-1H-indazol-1-yl)propanoic acid; 3-(Isoquinolin-6-yl)-3-(5-(2-(5,6,7,8-tetrahydro-1,8-naphthyridin-2-yl)et-hoxy)-1H-indazol-1-yl)propanoic acid; 3-(Pyrido[2,3-b]pyrazin-7-yl)-3-(5-(2-(5,6,7,8-tetrahydro-1,8-naphthyridi-n-2-yl)ethoxy)-1H-indazol-1-yl)propanoic acid; 3-(1,8-Naphthyridin-2-yl)-3-(5-(2-(5,6,7,8-tetrahydro-1,8-naphthyridin-2-yl)ethoxy)-1H-indazol-1-yl)propanoic acid; 3-(3,4-Dihydro-2H-pyrido[3,2-b][1,4]oxazin-7-yl)-3-(5-(2-(5,6,7,8-tetrahy-dro-1,8-naphthyridin-2-yl)ethoxy)-1H-indazol-1-yl)propanoic acid; (S)-2-(((Benzyloxy)carbonyl)amino)-3-(5-((5,6,7,8-tetrahydro-1,8-naphthyr-idin-2-yl)methoxy)-2H-indazol-2-yl)propanoic acid; (R)-3-(5-(Hydroxymethyl)pyridin-3-yl)-3-(5-(2-(5,6,7,8-tetrahydro-1,8-nap-hthyridin-2-yl)ethoxy)-1H-indazol-1-yl)propanoic acid; (S)-3-(5-(Hydroxymethyl)pyridin-3-yl)-3-(5-(2-(5,6,7,8-tetrahydro-1,8-nap-hthyridin-2-yl)ethoxy)-1H-indazol-1-yl)propanoic acid; 3-(1,8-Naphthyridin-3-yl)-3-(5-(2-(5,6,7,8-tetrahydro-1,8-naphthyridin-2-yl)ethoxy)-1H-indazol-1-yl)propanoic acid; 3-(5-(2-Oxopyrrolidin-1-yl)pyridin-3-yl)-3-(5-(2-(5,6,7,8-tetrahydro-1,8-naphthyridin-2-yl)ethoxy)-1H-indazol-1-yl)propanoic acid; 3-(5-(2-(5,6,7,8-Tetrahydro-1,8-naphthyridin-2-yl)ethoxy)-1H-indazol-1-yl-)-3-(1-(tetrahydro-2H-pyran-2-yl)pyrazolo[3,4-b]pyridin-5-yl)propanoic acid; 3-(5-(1,3-Dioxolan-2-yl)pyridin-3-yl)-3-(5-(2-(5,6,7,8-tetrahydro-1-,8-naphthyridin-2-yl)ethoxy)-1H-indazol-1-yl)propanoic acid; 3-(5-((Dimethylamino)methyl)pyridin-3-yl)-3-(5-(2-(5,6,7,8-tetrahydro-1,8-naphthyridin-2-yl)ethoxy)-1H-indazol-1-yl)propanoic acid; 3-(1H-Pyrazolo[3,4-b]pyridin-5-yl)-3-(5-(2-(5,6,7,8-tetrahydro-1,8-naphth-yridin-2-yl)ethoxy)-1H-indazol-1-yl)propanoic acid; (S)-3-(2-Ethoxypyrimidin-5-yl)-3-(5-(2-(5,6,7,8-tetrahydro-1,8-naphthyrid-in-2-yl)ethoxy)-1H-indazol-1-yl)propanoic acid; (R)-3-(2-Ethoxypyrimidin-5-yl)-3-(5-(2-(5,6,7,8-tetrahydro-1,8-naphthyrid-in-2-yl)ethoxy)-1H-indazol-1-yl)propanoic acid; 3-(2,3-Dihydro-[1,4]dioxino[2,3-b]pyridin-7-yl)-3-(5-(2-(5,6,7,8-tetrahyd-ro-1,8-naphthyridin-2-yl)ethoxy)-1H-indazol-1-yl)propanoic acid; 3-(Benzo[d]thiazol-6-yl)-3-(5-(2-(5,6,7,8-tetrahydro-1,8-naphthyridin-2-y-l)ethoxy)-1H-indazol-1-yl)propanoic acid; 3-(2-Morpholinopyrimidin-5-yl)-3-(5-(2-(5,6,7,8-tetrahydro-1,8-naphthyrid-in-2-yl)ethoxy)-1H-indazol-1-yl)propanoic acid; 3-(2-(Methylamino)pyrimidin-5-yl)-3-(5-(2-(5,6,7,8-tetrahydro-1,8-naphthy-ridin-2-yl)ethoxy)-1H-indazol-1-yl)propanoic acid; 3-(6-Methoxypyridin-3-yl)-3-(5-(3-(5,6,7,8-tetrahydro-1,8-naphthyridin-2-yl)propyl)-1H-pyrazolo[4,3-b]pyridin-1-yl)propanoic acid; 3-(6-Methoxypyridazin-3-yl)-3-(5-(2-(5,6,7,8-tetrahydro-1,8-naphthyridin-2-yl)ethoxy)-1H-indazol-1-yl)propanoic acid; 3-([1,2,4]Triazolo[4,3-a]pyridin-6-yl)-3-(5-(2-(5,6,7,8-tetrahydro-1,8-na-phthyridin-2-yl)ethoxy)-1H-indazol-1-yl)propanoic acid; 3-(5,6,7,8-Tetrahydro-1,8-naphthyridin-2-yl)-3-(5-(2-(5,6,7,8-tetrahydro-1,8-naphthyridin-2-yl)ethoxy)-1H-indazol-1-yl)propanoic acid; 3-(1-Methyl-1H-pyrazolo[3,4-b]pyridin-5-yl)-3-(5-(2-(5,6,7,8-tetrahydro-1-,8-naphthyridin-2-yl)ethoxy)-1H-indazol-1-yl)propanoic acid; (S)-3-(2-Methoxypyrimidin-5-yl)-3-(6-(2-(5,6,7,8-tetrahydro-1,8-naphthyri-din-2-yl)ethyl)-2H-indazol-2-yl)propanoic acid; 3-(2-(Azetidin-1-yl)pyrimidin-5-yl)-3-(5-(2-(5,6,7,8-tetrahydro-1,8-napht-hyridin-2-yl)ethoxy)-1H-indazol-1-yl)propanoic acid; 3-(2-Methyl-2H-pyrazolo[3,4-b]pyridin-5-yl)-3-(5-(2-(5,6,7,8-tetrahydro-1-,8-naphthyridin-2-yl)ethoxy)-1H-indazol-1-yl)propanoic acid; 3-(5-(Oxazol-5-yl)pyridin-3-yl)-3-(5-(2-(5,6,7,8-tetrahydro-1,8-naphthyri-din-2-yl)ethoxy)-1H-indazol-1-yl)propanoic acid; 3-(7-Ethyl-5,6,7,8-tetrahydroimidazo[1,2-a]pyrazin-3-yl)-3-(5-(2-(5,6,7,8- -tetrahydro-1,8-naphthyridin-2-yl)ethoxy)-1H-indazol-1-yl)propanoic acid; 3-(5-(((tert-Butoxycarbonyl)amino)methyl)pyridin-3-yl)-3-(5-(2-(5,6,7,8-tetrahydro-1,8- -naphthyridin-2-yl)ethoxy)-1H-indazol-1-yl)propanoic acid; 3-(5-Morpholinopyridin-3-yl)-3-(5-(2-(5,6,7,8-tetrahydro-1,8-naphthyridin-2-yl)ethoxy)-1H-indazol-1-yl)propanoic acid; 3-(5-(Methylsulfonyl)pyridin-3-yl)-3-(5-(2-(5,6,7,8-tetrahydro-1,8-naphth-yridin-2-yl)ethoxy)-1H-indazol-1-yl)propanoic acid; 3-(3-Methyl-3H-imidazo[4,5-b]pyridin-6-yl)-3-(5-(2-(5,6,7,8-tetrahydro-1,-8-naphthyridin-2-yl)ethoxy)-1H-indazol-1-yl)propanoic acid; 3-(5-(Pyrrolidin-1-yl)pyridin-3-yl)-3-(5-(2-(5,6,7,8-tetrahydro-1,8-napht-hyridin-2-yl)ethoxy)-1H-indazol-1-yl)propanoic acid; (R)-3-(Pyrido[2,3-b]pyrazin-7-yl)-3-(5-(2-(5,6,7,8-tetrahydro-1,8-naphthy-ridin-2-yl)ethoxy)-1H-indazol-1-yl)propanoic acid; (S)-3-(Pyrido[2,3-b]pyrazin-7-yl)-3-(5-(2-(5,6,7,8-tetrahydro-1,8-naphthy-ridin-2-yl)ethoxy)-1H-indazol-1-yl)propanoic acid; 3-([1,2,4]Triazolo[4,3-a]pyridin-7-yl)-3-(5-(2-(5,6,7,8-tetrahydro-1,8-na-phthyridin-2-yl)ethoxy)-1H-indazol-1-yl)propanoic acid; 3-(5-(Aminomethyl)pyridin-3-yl)-3-(5-(2-(5,6,7,8-tetrahydro-1,8-naphthyri-din-2-yl)ethoxy)-1H-indazol-1-yl)propanoic acid; 3-([1,3]Dioxolo[4,5-b]pyridin-6-yl)-3-(5-(2-(5,6,7,8-tetrahydro-1,8-napht-hyridin-2-yl)ethoxy)-1H-indazol-1-yl)propanoic acid; 3-(5-(1,3-Dioxolan-2-yl)-6-methoxypyridin-3-yl)-3-(5-(2-(5,6,7,8-tetrahyd-ro-1,8-naphthyridin-2-yl)ethoxy)-1H-indazol-1-yl)propanoic acid; 3-(6-(1H-Pyrazol-1-yl)pyridin-2-yl)-3-(5-(2-(5,6,7,8-tetrahydro-1,8-napht-hyridin-2-yl)ethoxy)-1H-indazol-1-yl)propanoic acid; 3-(1-(Pyridin-4-yl)-1H-pyrazol-4-yl)-3-(5-(2-(5,6,7,8-tetrahydro-1,8-naph-thyridin-2-yl)ethoxy)-1H-indazol-1-yl)propanoic acid; 3-(2-Methyl-2H-pyrazolo[4,3-b]pyridin-6-yl)-3-(5-(2-(5,6,7,8-tetrahydro-1-,8-naphthyridin-2-yl)ethoxy)-1H-indazol-1-yl)propanoic acid; 3-(1-Methyl-1H-pyrazolo[4,3-b]pyridin-6-yl)-3-(5-(2-(5,6,7,8-tetrahydro-1-,8-naphthyridin-2-yl)ethoxy)-1H-indazol-1-yl)propanoic acid; (R)-3-(5-(1,3-dioxolan-2-yl)pyridin-3-yl)-3-(5-(2-(5,6,7,8-tetrahydro-1,8- -naphthyridin-2-yl)ethoxy)-1H-indazol-1-yl)propanoic acid; (S)-3-(5-(1,3-dioxolan-2-yl)pyridin-3-yl)-3-(5-(2-(5,6,7,8-tetrahydro-1,8- -naphthyridin-2-yl)ethoxy)-1H-indazol-1-yl)propanoic acid; 3-(1-methyl-1H-imidazo[4,5-b]pyridin-6-yl)-3-(5-(2-(5,6,7,8-tetrahydro-1,-8-naphthyridin-2-yl)ethoxy)-1H-indazol-1-yl)propanoic acid; 3-(5-(1H-pyrazol-5-yl)pyridin-3-yl)-3-(5-(2-(5,6,7,8-tetrahydro-1,8-napht-hyridin-2-yl)ethoxy)-1H-indazol-1-yl)propanoic acid; 3-(6-morpholinopyrazin-2-yl)-3-(5-(2-(5,6,7,8-tetrahydro-1,8-naphthyridin-2-yl)ethoxy)-1H-indazol-1-yl)propanoic acid; 3-(5-(1-methyl-1H-pyrazol-4-yl)pyridin-3-yl)-3-(5-(2-(5,6,7,8-tetrahydro-1,8-naphthyridin-2-yl)ethoxy)-1H-indazol-1-yl)propanoic acid; 3-(5-(2-hydroxypropan-2-yl)pyridin-3-yl)-3-(5-(2-(5,6,7,8-tetrahydro-1,8-naphthyridin-2-yl)ethoxy)-1H-indazol-1-yl)propanoic acid; (R)-3-(5-(2-hydroxypropan-2-yl)pyridin-3-yl)-3-(5-(2-(5,6,7,8-tetrahydro-1,8-naphthyridin-2-yl)ethoxy)-1H-indazol-1-yl)propanoic acid; (S)-3-(5-(2-hydroxypropan-2-yl)pyridin-3-yl)-3-(5-(2-(5,6,7,8-tetrahydro-1,8-naphthyridin-2-yl)ethoxy)-1H-indazol-1-yl)propanoic acid; (R)-3-(2-methoxypyrimidin-5-yl)-3-(5-(2-(5,6,7,8-tetrahydro-1,8-naphthyri-din-2-yl)ethoxy)-1H-indazol-1-yl)propanoic acid; (S)-3-(2-methoxypyrimidin-5-yl)-3-(5-(2-(5,6,7,8-tetrahydro-1,8-naphthyri-din-2-yl)ethoxy)-1H-indazol-1-yl)propanoic acid; 3-(6-methoxypyrazin-2-yl)-3-(5-(2-(5,6,7,8-tetrahydro-1,8-naphthyridin-2-yl)ethoxy)-1H-indazol-1-yl)propanoic acid; (R)-3-(5-(2-oxopyrrolidin-1-yl)pyridin-3-yl)-3-(5-(2-(5,6,7,8-tetrahydro-1,8-naphthyridin-2-yl)ethoxy)-1H-indazol-1-yl)propanoic acid; (S)-3-(5-(2-oxopyrrolidin-1-yl)pyridin-3-yl)-3-(5-(2-(5,6,7,8-tetrahydro-1,8-naphthyridin-2-yl)ethoxy)-1H-indazol-1-yl)propanoic acid; 3-(5-(morpholine-4-carbonyl)pyridin-3-yl)-3-(5-(2-(5,6,7,8-tetrahydro-1,8-naphthyridin-2-yl)ethoxy)-1H-indazol-1-yl)propanoic acid; 3-(5-(dimethylcarbamoyl)pyridin-3-yl)-3-(5-(2-(5,6,7,8-tetrahydro-1,8-nap-hthyridin-2-yl)ethoxy)-1H-indazol-1-yl)propanoic acid; 3-(5-(4-methylpiperazine-1-carbonyl)pyridin-3-yl)-3-(5-(2-(5,6,7,8-tetrah-ydro-1,8-naphthyridin-2-yl)ethoxy)-1H-indazol-1-yl)propanoic acid; 3-(5-cyclopropylpyridin-3-yl)-3-(5-(2-(5,6,7,8-tetrahydro-1,8-naphthyridi-n-2-yl)ethoxy)-1H-indazol-1-yl)propanoic acid; 3-(5-(4-methylpiperazin-1-yl)pyridin-3-yl)-3-(5-(2-(5,6,7,8-tetrahydro-1,-8-naphthyridin-2-yl)ethoxy)-1H-indazol-1-yl)propanoic acid; 3-(5-(azetidine-1-carbonyl)pyridin-3-yl)-3-(5-(2-(5,6,7,8-tetrahydro-1,8-naphthyridin-2-yl)ethoxy)-1H-indazol-1-yl)propanoic acid; 3-(5-((2-(dimethylamino)ethyl)carbamoyl)pyridin-3-yl)-3-(5-(2-(5,6,7,8-te-trahydro-1,8-naphthyridin-2-yl)ethoxy)-1H-indazol-1-yl)propanoic acid; (S)-3-(2-methylpyrimidin-5-yl)-3-(5-(2-(5,6,7,8-tetrahydro-1,8-naphthyrid-in-2-yl)ethoxy)-1H-indazol-1-yl)propanoic acid; (R)-3-(2-methylpyrimidin-5-yl)-3-(5-(2-(5,6,7,8-tetrahydro-1,8-naphthyrid-in-2-yl)ethoxy)-1H-indazol-1-yl)propanoic acid; 3-(5-(1H-pyrazol-1-yl)pyridin-3-yl)-3-(5-(2-(5,6,7,8-tetrahydro-1,8-napht-hyridin-2-yl)ethoxy)-1H-indazol-1-yl)propanoic acid; 3-(5-(2-(5,6,7,8-tetrahydro-1,8-naphthyridin-2-yl)ethoxy)-1H-indazol-1-yl-)hexanoic acid; 3-(5-(dimethylamino)pyridin-3-yl)-3-(5-(2(5,6,7,8-tetrahydro-1,8-naphthyr-idin-2-yl)ethoxy)-1H-indazol-1-yl)propanoic acid; 3-cyclohexyl-3-(5-(2-(5,6,7,8-tetrahydro-1,8-naphthyridin-2-yl)ethoxy)-1H- -indazol-1-yl)propanoic acid; (R)-3-(5-(methylsulfonyl)pyridin-3-yl)-3-(5-(2-(5,6,7,8-tetrahydro-1,8-na-phthyridin-2-yl)ethoxy)-1H-indazol-1-yl)propanoic acid; (S)-3-(5-(methyl sulfonyl)pyridin-3-yl)-3-(5-(2-(5,6,7,8-tetrahydro-1,8-naphthyridin-2-yl)-ethoxy)-1H-indazol-1-yl)propanoic acid; 3-(5-(((methoxycarbonyl)amino)methyl)pyridin-3-yl)-3-(5-(2-(5,6,7,8-tetra-hydro-1,8-naphthyridin-2-yl)ethoxy)-1H-indazol-1-yl)propanoic acid; (R)-3-(5-(((methoxycarbonyl)amino)methyl)pyridin-3-yl)-3-(5-(2-(5,6,7,8-t-etrahydro-1,8-naphthyridin-2-yl)ethoxy)-1H-indazol-1-yl)propanoic acid; (S)-3-(5-(((methoxycarbonyl)amino)methyl)pyridin-3-yl)-3-(5-(2-(5,6,7,8-t-etrahydro-1,8-naphthyridin-2-yl)ethoxy)-1H-indazol-1-yl)propanoic acid; 3-(5-(methylsulfonamidomethyl)pyridin-3-yl)-3-(5-(2-(5,6,7,8-tetrahydro-1-,8-naphthyridin-2-yl)ethoxy)-1H-indazol-1-yl)propanoic acid; 3-(5-(acetamidomethyl)pyridin-3-yl)-3-(5-(2-(5,6,7,8-tetrahydro-1,8-napht-hyridin-2-yl)ethoxy)-1H-indazol-1-yl)propanoic acid; 3-(5-(2-(5,6,7,8-Tetrahydro-1,8-naphthyridin-2-yl)ethoxy)-1H-indazol-1-yl-)-3-(5,6,7,8-tetrahydro-1,8-naphthyridin-3-yl)propanoic acid; 4-((6-(2-Carboxy-1-(5-(2-(5,6,7,8-tetrahydro-1,8-naphthyridin-2-yl)ethoxy-)-1H-indazol-1-yl)ethyl)pyrazin-2-yl)amino)butanoic acid; 3-(6-Methoxypyridin-3-yl)-3-(4-(2-(5,6,7,8-tetrahydro-1,8-naphthyridin-2-yl)ethyl)-1H-indazol-1-yl)propanoic acid; (R)-3-(5-(2-((R)-7-methyl-5,6,7,8-tetrahydro-1,8-naphthyridin-2-yl)ethoxy-)-1H-indazol-1-yl)-3-(2-methylpyrimidin-5-yl)propanoic acid; (S)-3-(5-(2-((R)-7-methyl-5,6,7,8-tetrahydro-1,8-naphthyridin-2-yl)ethoxy-)-1H-indazol-1-yl)-3-(2-methylpyrimidin-5-yl)propanoic acid; (R)-3-(5-(2-((S)-7-methyl-5,6,7,8-tetrahydro-1,8-naphthyridin-2-yl)ethoxy-)-1H-indazol-1-yl)-3-(2-methylpyrimidin-5-yl)propanoic acid; (S)-3-(5-(2-((S)-7-methyl-5,6,7,8-tetrahydro-1,8-naphthyridin-2-yl)ethoxy-)-1H-indazol-1-yl)-3-(2-methylpyrimidin-5-yl)propanoic acid; 3-(6-Methoxypyridin-3-yl)-3-(5-(2-(1-methyl-1,2,3,4-tetrahydropyrido[2,3-b]pyrazin-6-yl)ethoxy)-1H-indazol-1-yl)propanoic acid; (S)-3-(5-(2-(3,4-dihydro-2H-pyrido[3,2-b][1,4]oxazin-6-yl)ethoxy)-1H-inda-zol-1-yl)-3-(6-methoxypyridin-3-yl)propanoic acid; and (R)-3-(5-(2-(3,4-dihydro-2H-pyrido[3,2-b][1,4]oxazin-6-yl)ethoxy)-1H-inda-zol-1-yl)-3-(6-methoxypyridin-3-yl)propanoic acid; or a pharmaceutically acceptable salt thereof. See US 2019/0256496, which is hereby incorporated by reference it its entirety.

In some embodiments, the integrin antagonist is (S)-3-(3-(2-methoxyethoxy)phenyl)-4-((R)-3-(2-(5,6,7,8-tetrahydro-1,8-nap-hthyridin-2-yl)ethyl)pyrrolidin-1-yl)butanoic acid; (S)-3-(3-((R)-2-methoxypropoxylphenyl)-4-((R)-3-(2-(5,6,7,8-tetrahydro-1,-8-naphthyridin-2-yl)ethyl)pyrrolidin-1-yl)butanoic acid; (S)-3-(3-((S)-2-methoxypropoxylphenyl)-4-4R)-3-(2-(5,6,7,8-tetrahydro-1,8- -naphthyridin-2-yl)ethyl)pyrrolidin-1-yl)butanoic acid; (S)-3-(3-(2-methoxy-2-methylpropoxy)phenyl)-4-((R)-3-(2-(5,6,7,8-tetrahydr-o-1,8-naphthyridin-2-yl)ethyl)pyrrolidin-1-yl)butanoic acid; 3-(3-(((S)-1-methoxypropan-2-yl)oxy)phenyl)-4-((R)-3-(2-(5,6,7,8-tetrahyd-ro-1,8-naphthyridin-2-yl)ethyl)pyrrolidin-1-yl)butanoic acid; (S)-4-((R)-3-(2-(5,6,7,8-tetrahydro-1,8-naphthyridin-2-yl)ethyl)pyrrolidi-n-1-yl)-3-(3-(((S)-tetrahydrofuran-3-yl)oxy)phenyl)butanoic acid; (S)-4-((R)-3-(2-(5,6,7,8-tetrahydro-1,8-naphthyridin-2-yl)ethyl)pyrrolidi-n-1-yl)-3-(3-(((R)-tetrahydrofuran-3-yl)oxy)phenyl)butanoic acid; (S)-3-(3,5-Bis(2-methoxyethoxy)phenyl)-4-((R)-3-(2-(5,6,7,8-tetrahydro-1,-8-naphthyridin-2-yl)ethyl)pyrrolidin-1-yl)butanoic acid; (3S)-4-((R)-3-(2-(5,6,7,8-Tetrahydro-1,8-naphthyridin-2-yl)ethyl)pyrrolid-in-1-yl)-3-(3-(tetrahydrofuran-3-yl)phenyebutanoic acid; (3S)-4-((R)-3-(2-(5,6,7,8-Tetrahydro-1,8-naphthyridin-2-yl)ethyl)pyrrolid-in-1-yl)-3-(3-(tetrahydrofuran-3-yl)phenyebutanoic acid; (S)-3-(3-((1-methoxy-2-methylpropan-2-yl)oxy)phenyl)-4-4R)-3-(2-(5,6,7,8-tetrahydro-1,8-naphthyridin-2-yl)ethyl)pyrrolidin-1-yl)butanoic acid; (S)-3-(3-(((R)-1-methoxypropan-2-yl)oxy)phenyl)-4-((R)-3-(2-(5,6,7,8-tetr-ahydro-1,8-naphthyridin-2-yl)ethyl)pyrrolidin-1-yl)butanoic acid; (S)-3-(3-(2-Isopropoxyethoxy)phenyl)-4-((R)-3-(2-(5,6,7,8-tetrahydro-1,8-naphthyridin-2-yl)ethyl)pyrrolidin-1-yl)butanoic acid; (S)-4-((R)-3-(2-(5,6,7,8-Tetrahydro-1,8-naphthyridin-2-yl)ethyl)pyrrolidi-n-1-yl)-3-(3-(((R)-tetrahydrofuran-2-yl)methoxy)phenyl)butanoic acid; (S)-4-((R)-3-(2-(5,6,7,8-tetrahydro-1,8-naphthyridin-2-yl)ethyl)pyrrolidi-n-1-yl)-3-(3-(((S)-tetrahydrofuran-2-yl)methoxy)phenyl)butanoic acid; (S)-3-(2-Fluoro-5-(2-methoxyethoxy)phenyl)-4-((R)-3-(2-(5,6,7,8-tetrahydr-o-1,8-naphthyridin-2-yl)ethyl)pyrrolidin-1-yl)butanoic acid; 3-(3-((1,3-Dimethoxypropan-2-yeoxy)phenyl)-4-((R)-3-(2-(5,6,7,8-tetrahydr-o-1,8-naphthyridin-2-yl)ethyl)pyrrolidin-1-yl)butanoic acid; 3-(3-(2-Fluoroethoxy)-5-(2-methoxyethoxy)phenyl)-4-((R)-3-(2-(5,6,7,8-tet-rahydro-1,8-naphthyridin-2-yl)ethyl)pyrrolidin-1-yl)butanoic acid; 3-(3-(3-Methoxypropoxy)phenyl)-4-((R)-3-(2-(5,6,7,8-tetrahydro-1,8-naphth-yridin-2-yl)ethyl)pyrrolidin-1-yl)butanoic acid; or 3-(3-(Oxetan-3-ylmethoxy)phenyl)-4-((R)-3-(2-(5,6,7,8-tetrahydro-1,8-naph-thyridin-2-yl)ethyl)pyrrolidin-1-yl)butanoic acid; or a pharmaceutically acceptable salt thereof. See US 2019/0112306, which is hereby incorporated by reference it its entirety.

In some embodiments, the integrin antagonist has the structure

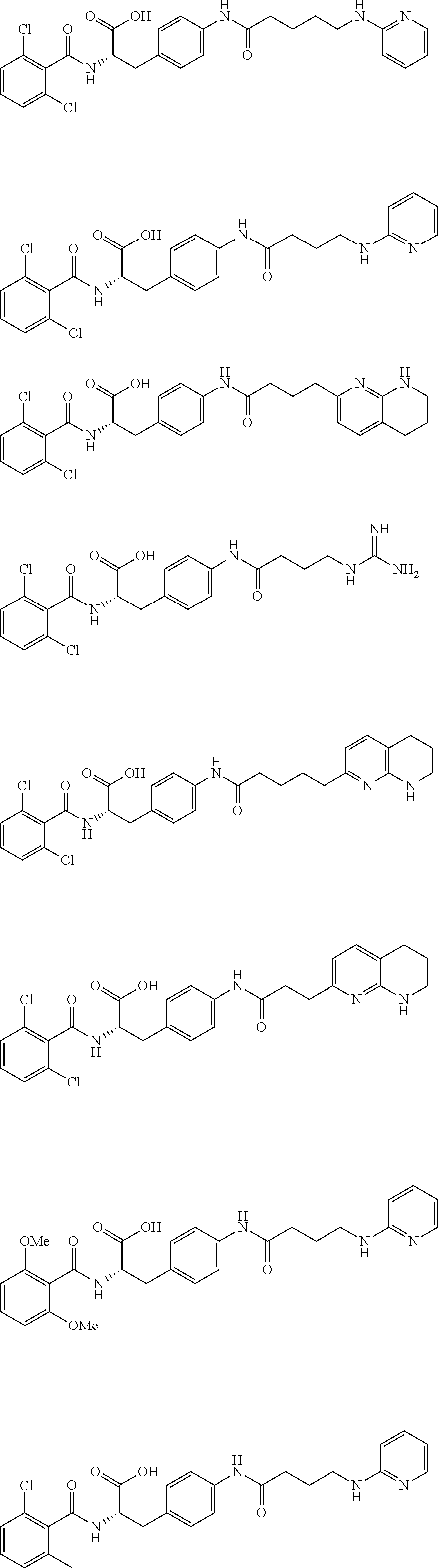

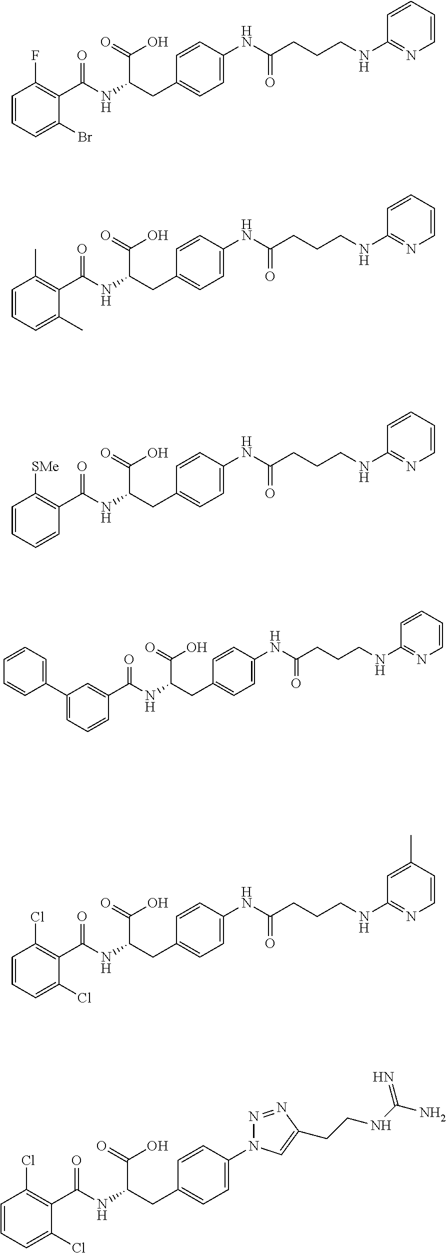 [SMILES: O=C(CCCNc1ccccn1)Nc1ccc(C[C@H](NC(=O)c2c(F)cccc2Br)C(=O)O)cc1 ; Cc1cccc(C)c1C(=O)N[C@@H](Cc1ccc(NC(=O)CCCNc2ccccn2)cc1)C(=O)O ; CSc1ccccc1C(=O)N[C@@H](Cc1ccc(NC(=O)CCCNc2ccccn2)cc1)C(=O)O ; O=C(CCCNc1ccccn1)Nc1ccc(C[C@H](NC(=O)c2cccc(-c3ccccc3)c2)C(=O)O)cc1 ; Cc1ccnc(NCCCC(=O)Nc2ccc(C[C@H](NC(=O)c3c(Cl)cccc3Cl)C(=O)O)cc2)c1 ; N=C(N)NCCc1cn(-c2ccc(C[C@H](NC(=O)c3c(Cl)cccc3Cl)C(=O)O)cc2)nn1]

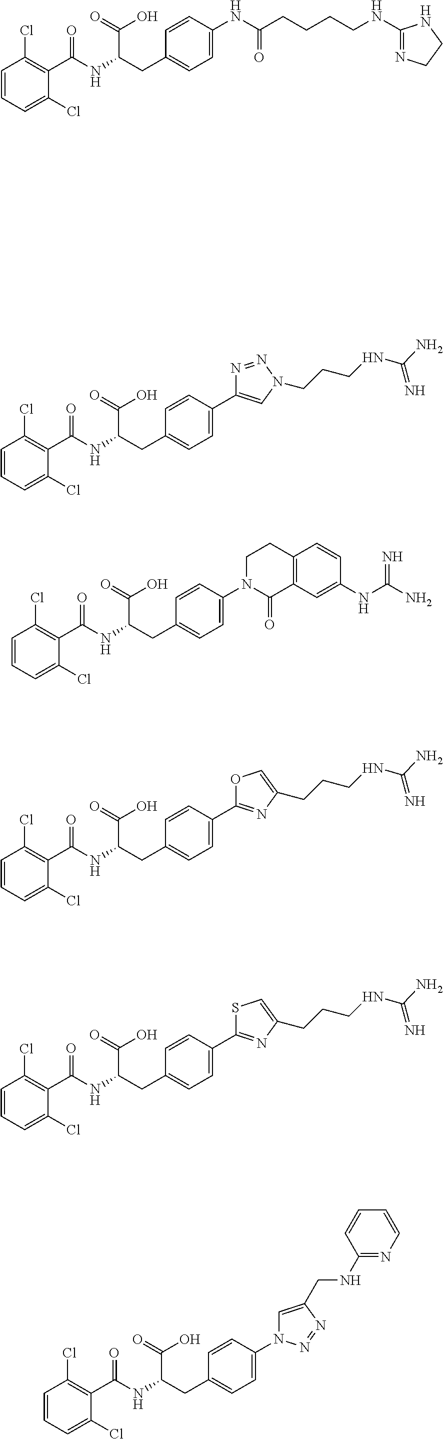 [SMILES: O=C(CCCCNC1=NCCN1)Nc1ccc(C[C@H](NC(=O)c2c(Cl)cccc2Cl)C(=O)O)cc1 ; N=C(N)NCCCn1cc(-c2ccc(C[C@H](NC(=O)c3c(Cl)cccc3Cl)C(=O)O)cc2)nn1 ; N=C(N)Nc1ccc2c(c1)C(=O)N(c1ccc(C[C@H](NC(=O)c3c(Cl)cccc3Cl)C(=O)O)cc1)CC2 ; N=C(N)NCCCc1coc(-c2ccc(C[C@H](NC(=O)c3c(Cl)cccc3Cl)C(=O)O)cc2)n1 ; N=C(N)NCCCc1csc(-c2ccc(C[C@H](NC(=O)c3c(Cl)cccc3Cl)C(=O)O)cc2)n1 ; O=C(N[C@@H](Cc1ccc(-n2cc(CNc3ccccn3)nn2)cc1)C(=O)O)c1c(Cl)cccc1Cl]

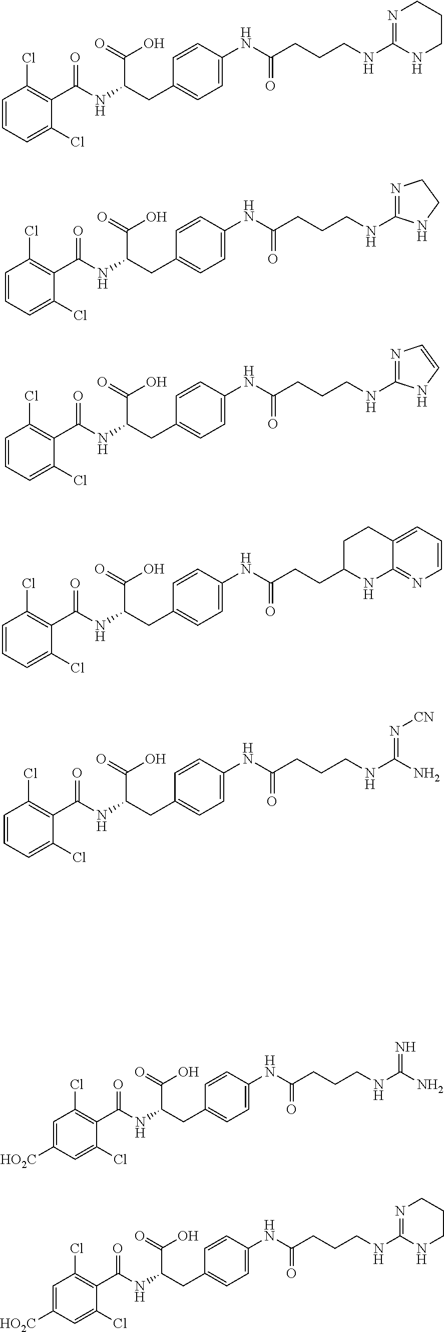

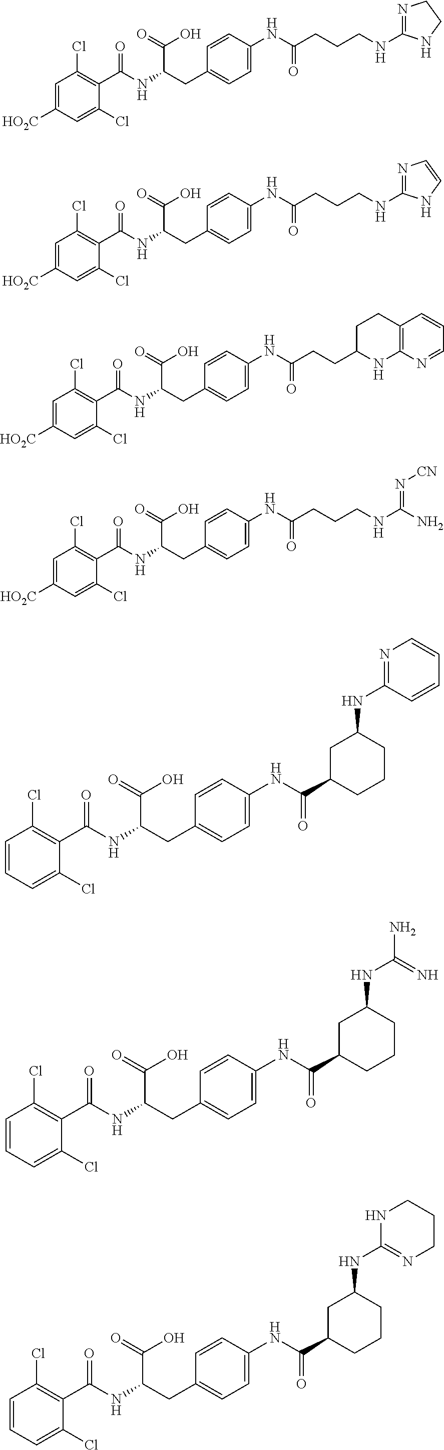

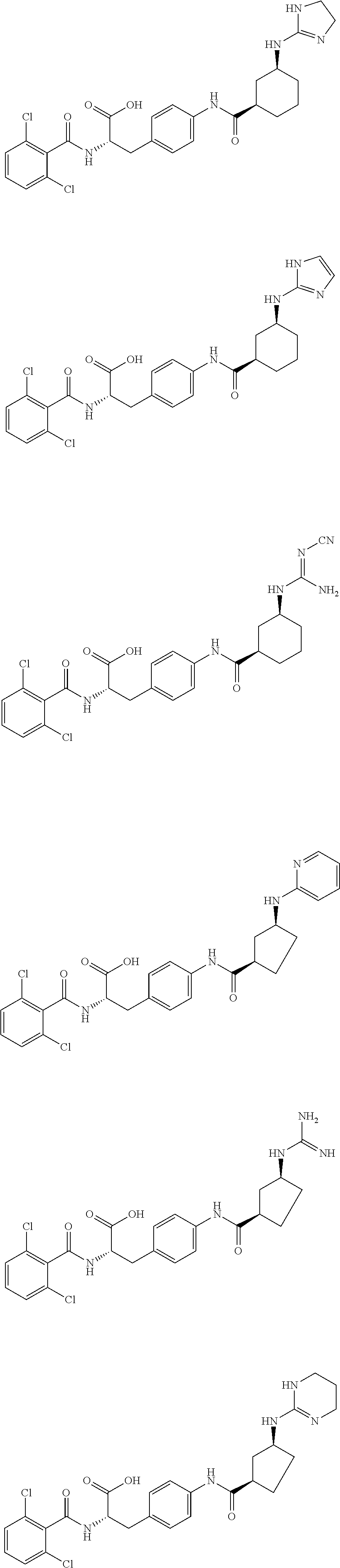 [SMILES: O=C(N[C@@H](Cc1ccc(NC(=O)[C@@H]2CCC[C@H](NC3=NCCN3)C2)cc1)C(=O)O)c1c(Cl)cccc1Cl ; O=C(N[C@@H](Cc1ccc(NC(=O)[C@@H]2CCC[C@H](Nc3ncc[nH]3)C2)cc1)C(=O)O)c1c(Cl)cccc1Cl ; N#C/N=C(\N)N[C@H]1CCC[C@@H](C(=O)Nc2ccc(C[C@H](NC(=O)c3c(Cl)cccc3Cl)C(=O)O)cc2)C1 ; O=C(N[C@@H](Cc1ccc(NC(=O)[C@@H]2CC[C@H](Nc3ccccn3)C2)cc1)C(=O)O)c1c(Cl)cccc1Cl ; N=C(N)N[C@H]1CC[C@@H](C(=O)Nc2ccc(C[C@H](NC(=O)c3c(Cl)cccc3Cl)C(=O)O)cc2)C1 ; O=C(N[C@@H](Cc1ccc(NC(=O)[C@@H]2CC[C@H](NC3=NCCCN3)C2)cc1)C(=O)O)c1c(Cl)cccc1Cl]

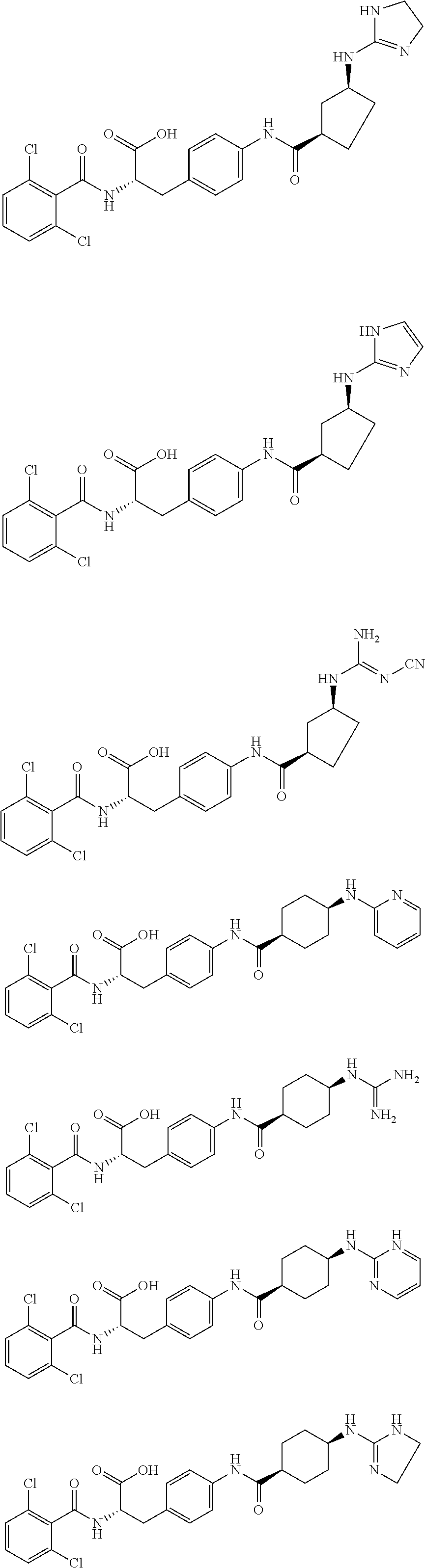

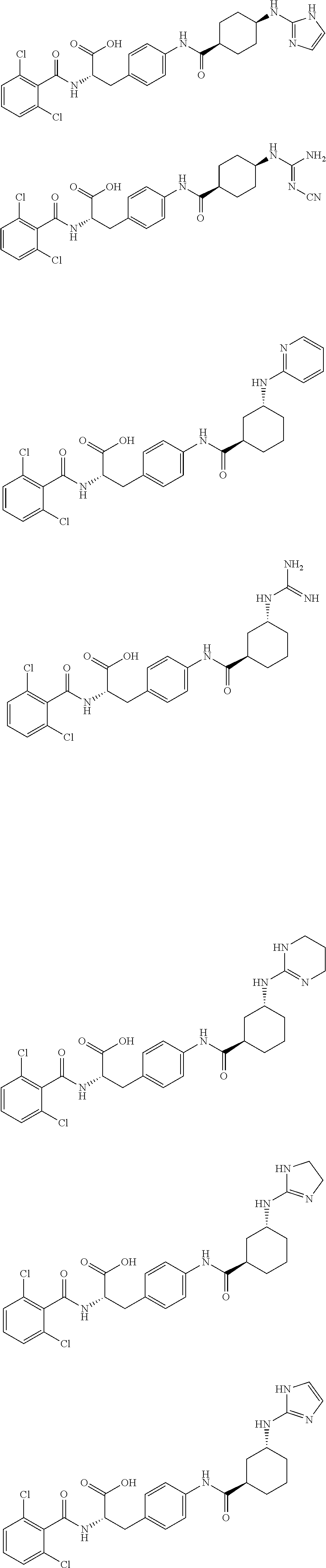

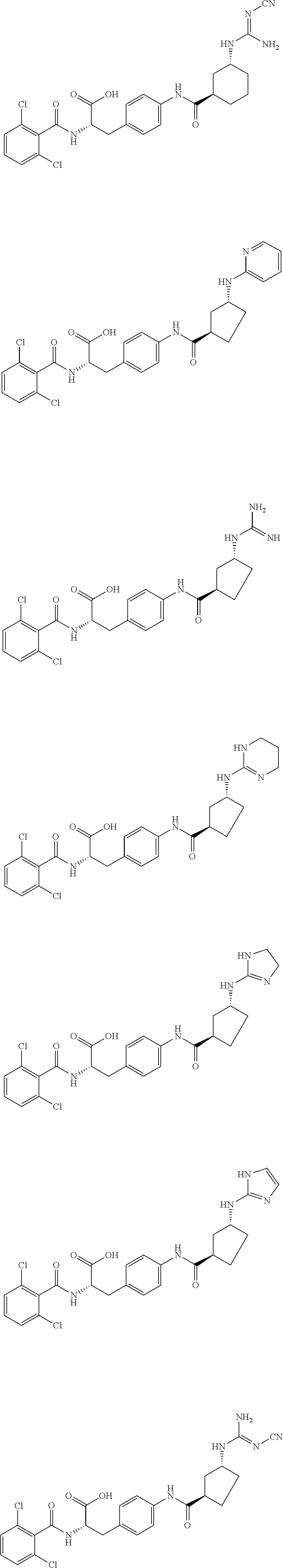

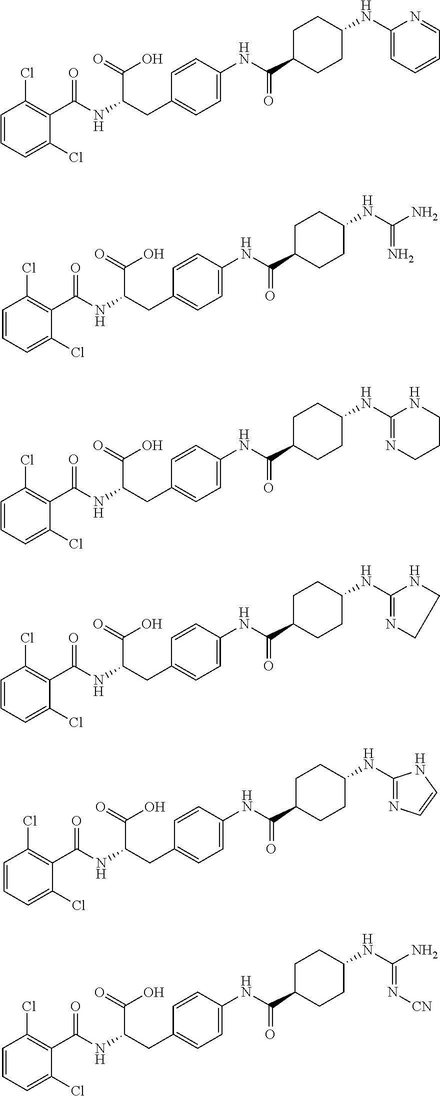 [SMILES: O=C(N[C@@H](Cc1ccc(NC(=O)[C@H]2CC[C@H](Nc3ccccn3)CC2)cc1)C(=O)O)c1c(Cl)cccc1Cl ; N=C(N)N[C@H]1CC[C@H](C(=O)Nc2ccc(C[C@H](NC(=O)c3c(Cl)cccc3Cl)C(=O)O)cc2)CC1 ; O=C(N[C@@H](Cc1ccc(NC(=O)[C@H]2CC[C@H](NC3=NCCCN3)CC2)cc1)C(=O)O)c1c(Cl)cccc1Cl ; O=C(N[C@@H](Cc1ccc(NC(=O)[C@H]2CC[C@H](NC3=NCCN3)CC2)cc1)C(=O)O)c1c(Cl)cccc1Cl ; O=C(N[C@@H](Cc1ccc(NC(=O)[C@H]2CC[C@H](Nc3ncc[nH]3)CC2)cc1)C(=O)O)c1c(Cl)cccc1Cl ; N#C/N=C(\N)N[C@H]1CC[C@H](C(=O)Nc2ccc(C[C@H](NC(=O)c3c(Cl)cccc3Cl)C(=O)O)cc2)CC1]

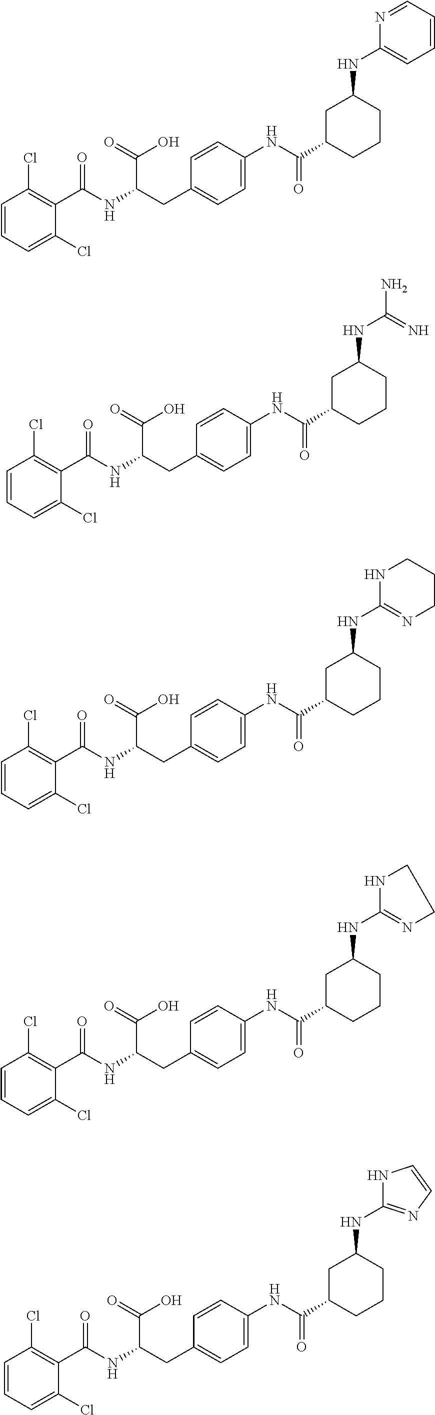 [SMILES: O=C(N[C@@H](Cc1ccc(NC(=O)[C@H]2CCC[C@H](Nc3ccccn3)C2)cc1)C(=O)O)c1c(Cl)cccc1Cl ; N=C(N)N[C@H]1CCC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CCC[C@H](NC3=NCCCN3)C2)cc1)C(=O)O)c1c(Cl)cccc1Cl ; O=C(N[C@@H](Cc1ccc(NC(=O)[C@H]2CCC[C@H](NC3=NCCN3)C2)cc1)C(=O)O)c1c(Cl)cccc1Cl ; O=C(N[C@@H](Cc1ccc(NC(=O)[C@H]2CCC[C@H](Nc3ncc[nH]3)C2)cc1)C(=O)O)c1c(Cl)cccc1Cl]

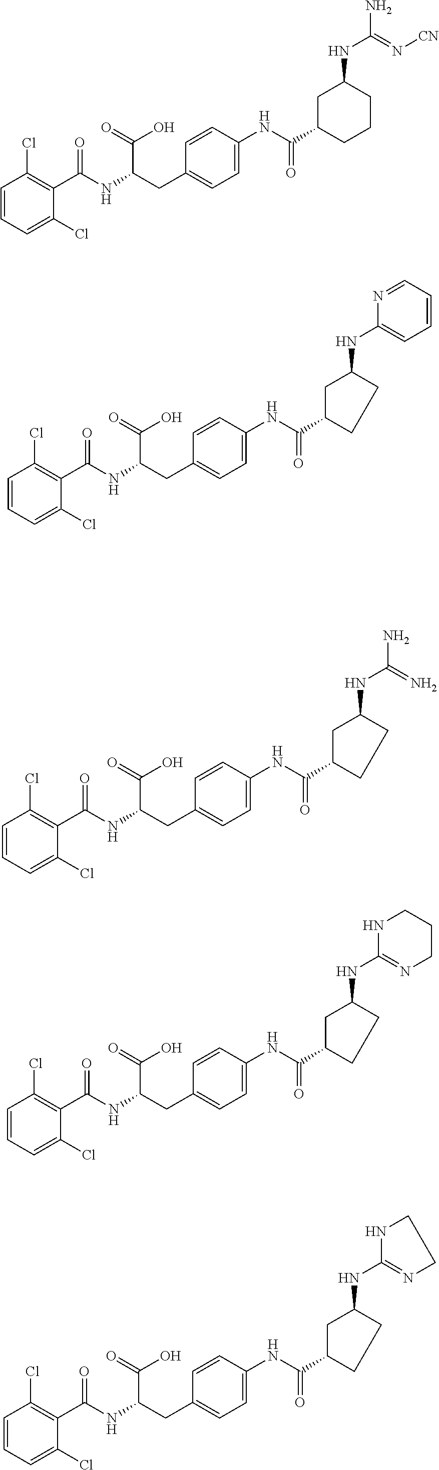 [SMILES: N#C/N=C(\N)N[C@H]1CCC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CC[C@H](Nc3ccccn3)C2)cc1)C(=O)O)c1c(Cl)cccc1Cl ; N=C(N)N[C@H]1CC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CC[C@H](NC3=NCCCN3)C2)cc1)C(=O)O)c1c(Cl)cccc1Cl ; O=C(N[C@@H](Cc1ccc(NC(=O)[C@H]2CC[C@H](NC3=NCCN3)C2)cc1)C(=O)O)c1c(Cl)cccc1Cl]

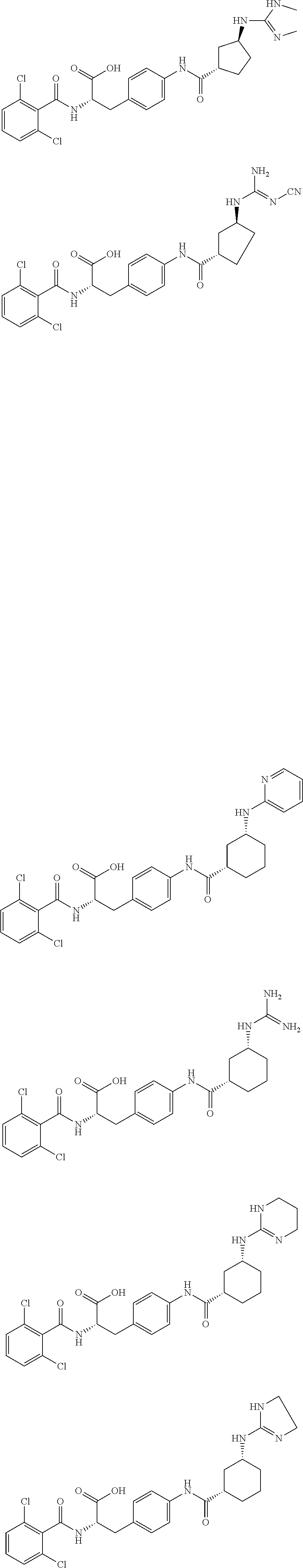 [SMILES: C/N=C(\NC)N[C@H]1CC[C@H](C(=O)Nc2ccc(C[C@H](NC(=O)c3c(Cl)cccc3Cl)C(=O)O)cc2)C1 ; N#C/N=C(\N)N[C@H]1CC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CCC[C@@H](Nc3ccccn3)C2)cc1)C(=O)O)c1c(Cl)cccc1Cl ; N=C(N)N[C@@H]1CCC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CCC[C@@H](NC3=NCCCN3)C2)cc1)C(=O)O)c1c(Cl)cccc1Cl ; O=C(N[C@@H](Cc1ccc(NC(=O)[C@H]2CCC[C@@H](NC3=NCCN3)C2)cc1)C(=O)O)c1c(Cl)cccc1Cl]

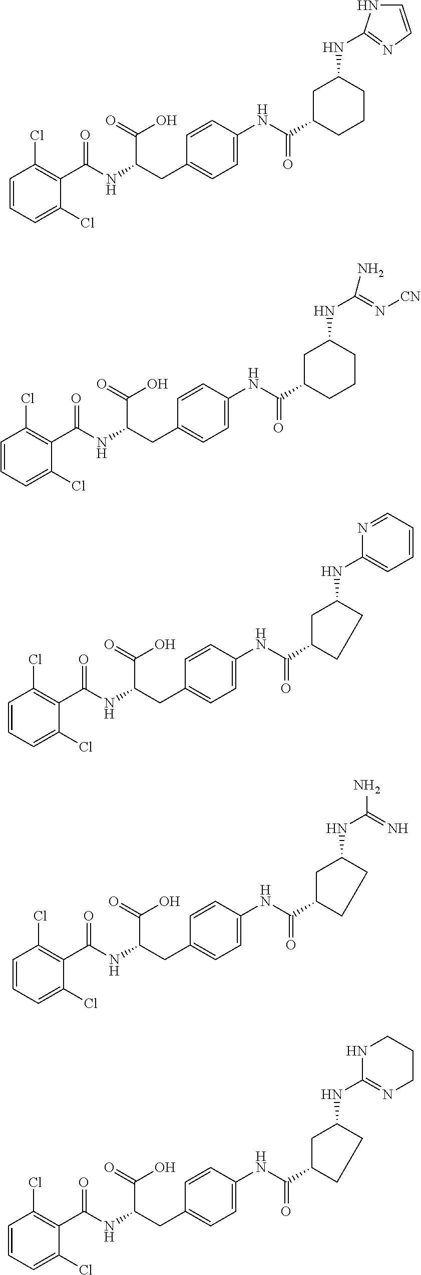 [SMILES: O=C(N[C@@H](Cc1ccc(NC(=O)[C@H]2CCC[C@@H](Nc3ncc[nH]3)C2)cc1)C(=O)O)c1c(Cl)cccc1Cl ; N#C/N=C(\N)N[C@@H]1CCC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CC[C@@H](Nc3ccccn3)C2)cc1)C(=O)O)c1c(Cl)cccc1Cl ; N=C(N)N[C@@H]1CC[C@H](C(=O)Nc2ccc(C[C@H](NC(=O)c3c(Cl)cccc3Cl)C(=O)O)cc2)C1 ; O=C(N[C@@H](Cc1ccc(NC(=O)[C@H]2CC[C@@H](NC3=NCCCN3)C2)cc1)C(=O)O)c1c(Cl)cccc1Cl]

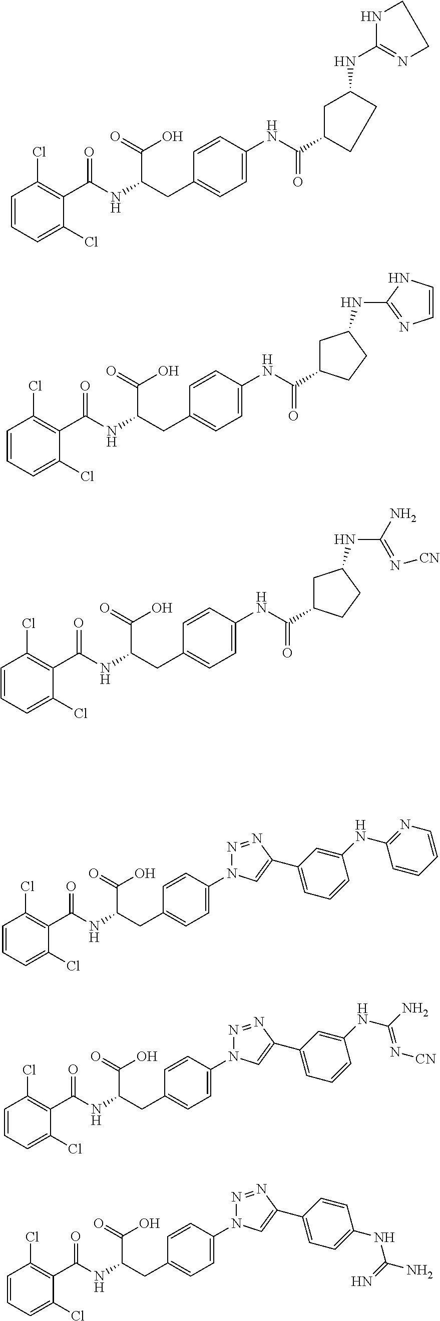 [SMILES: O=C(N[C@@H](Cc1ccc(NC(=O)[C@H]2CC[C@@H](NC3=NCCN3)C2)cc1)C(=O)O)c1c(Cl)cccc1Cl ; O=C(N[C@@H](Cc1ccc(NC(=O)[C@H]2CC[C@@H](Nc3ncc[nH]3)C2)cc1)C(=O)O)c1c(Cl)cccc1Cl ; N#C/N=C(\N)N[C@@H]1CC[C@H](C(=O)Nc2ccc(C[C@H](NC(=O)c3c(Cl)cccc3Cl)C(=O)O)cc2)C1 ; O=C(N[C@@H](Cc1ccc(-n2cc(-c3cccc(Nc4ccccn4)c3)nn2)cc1)C(=O)O)c1c(Cl)cccc1Cl ; N#C/N=C(\N)Nc1cccc(-c2cn(-c3ccc(C[C@H](NC(=O)c4c(Cl)cccc4Cl)C(=O)O)cc3)nn2)c1 ; N=C(N)Nc1ccc(-c2cn(-c3ccc(C[C@H](NC(=O)c4c(Cl)cccc4Cl)C(=O)O)cc3)nn2)cc1]

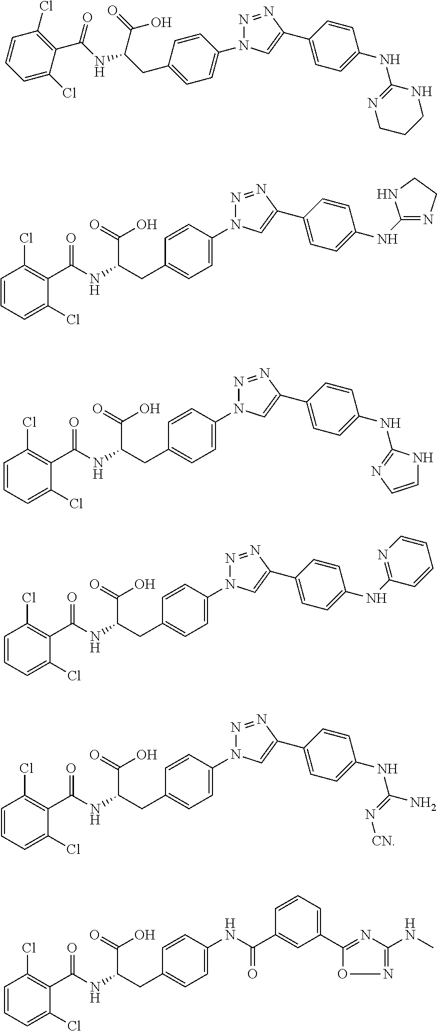 [SMILES: O=C(N[C@@H](Cc1ccc(-n2cc(-c3ccc(NC4=NCCCN4)cc3)nn2)cc1)C(=O)O)c1c(Cl)cccc1Cl ; O=C(N[C@@H](Cc1ccc(-n2cc(-c3ccc(NC4=NCCN4)cc3)nn2)cc1)C(=O)O)c1c(Cl)cccc1Cl ; O=C(N[C@@H](Cc1ccc(-n2cc(-c3ccc(Nc4ncc[nH]4)cc3)nn2)cc1)C(=O)O)c1c(Cl)cccc1Cl ; O=C(N[C@@H](Cc1ccc(-n2cc(-c3ccc(Nc4ccccn4)cc3)nn2)cc1)C(=O)O)c1c(Cl)cccc1Cl ; N#C/N=C(\N)Nc1ccc(-c2cn(-c3ccc(C[C@H](NC(=O)c4c(Cl)cccc4Cl)C(=O)O)cc3)nn2)cc1 ; CNc1noc(-c2cccc(C(=O)Nc3ccc(C[C@H](NC(=O)c4c(Cl)cccc4Cl)C(=O)O)cc3)c2)n1]

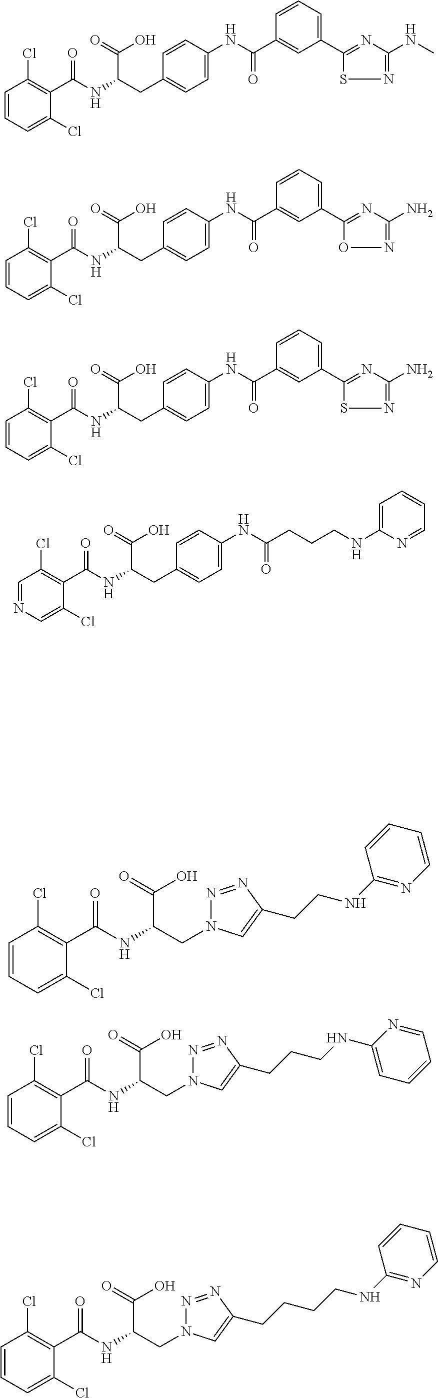

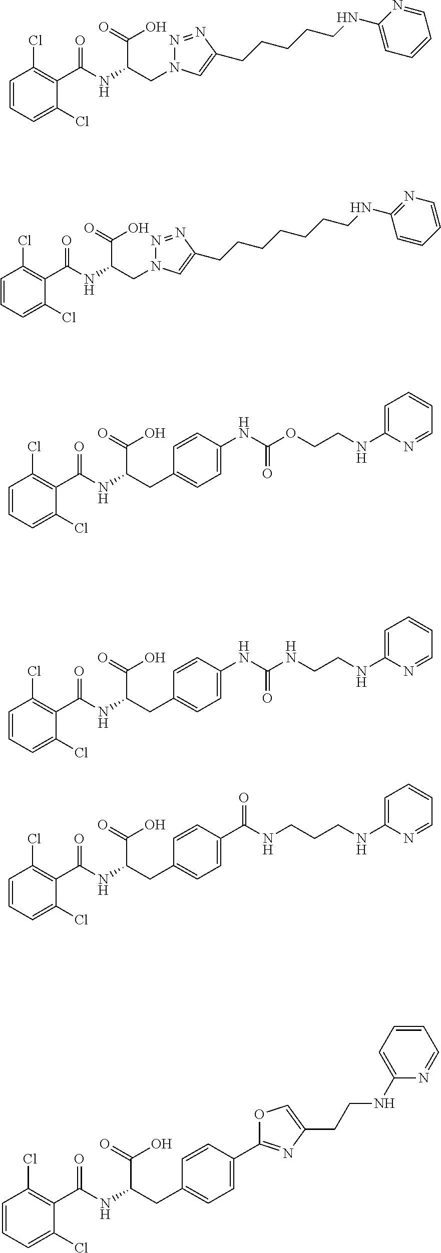 [SMILES: O=C(N[C@@H](Cn1cc(CCCCCNc2ccccn2)nn1)C(=O)O)c1c(Cl)cccc1Cl ; O=C(N[C@@H](Cn1cc(CCCCCCCNc2ccccn2)nn1)C(=O)O)c1c(Cl)cccc1Cl ; O=C(Nc1ccc(C[C@H](NC(=O)c2c(Cl)cccc2Cl)C(=O)O)cc1)OCCNc1ccccn1 ; O=C(NCCNc1ccccn1)Nc1ccc(C[C@H](NC(=O)c2c(Cl)cccc2Cl)C(=O)O)cc1 ; O=C(NCCCNc1ccccn1)c1ccc(C[C@H](NC(=O)c2c(Cl)cccc2Cl)C(=O)O)cc1 ; O=C(N[C@@H](Cc1ccc(-c2nc(CCNc3ccccn3)co2)cc1)C(=O)O)c1c(Cl)cccc1Cl]

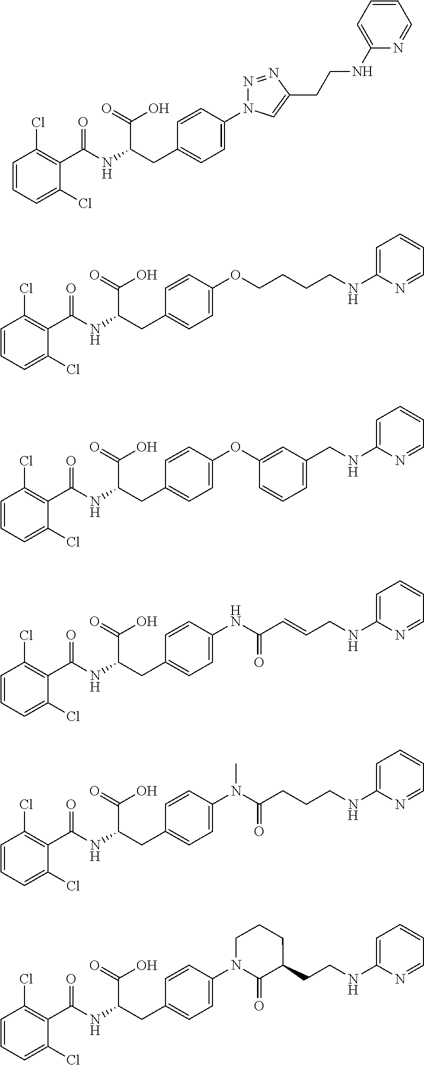 [SMILES: O=C(N[C@@H](Cc1ccc(-n2cc(CCNc3ccccn3)nn2)cc1)C(=O)O)c1c(Cl)cccc1Cl ; O=C(N[C@@H](Cc1ccc(OCCCCNc2ccccn2)cc1)C(=O)O)c1c(Cl)cccc1Cl ; O=C(N[C@@H](Cc1ccc(Oc2cccc(CNc3ccccn3)c2)cc1)C(=O)O)c1c(Cl)cccc1Cl ; O=C(/C=C/CNc1ccccn1)Nc1ccc(C[C@H](NC(=O)c2c(Cl)cccc2Cl)C(=O)O)cc1 ; CN(C(=O)CCCNc1ccccn1)c1ccc(C[C@H](NC(=O)c2c(Cl)cccc2Cl)C(=O)O)cc1 ; O=C(N[C@@H](Cc1ccc(N2CCC[C@@H](CCNc3ccccn3)C2=O)cc1)C(=O)O)c1c(Cl)cccc1Cl]

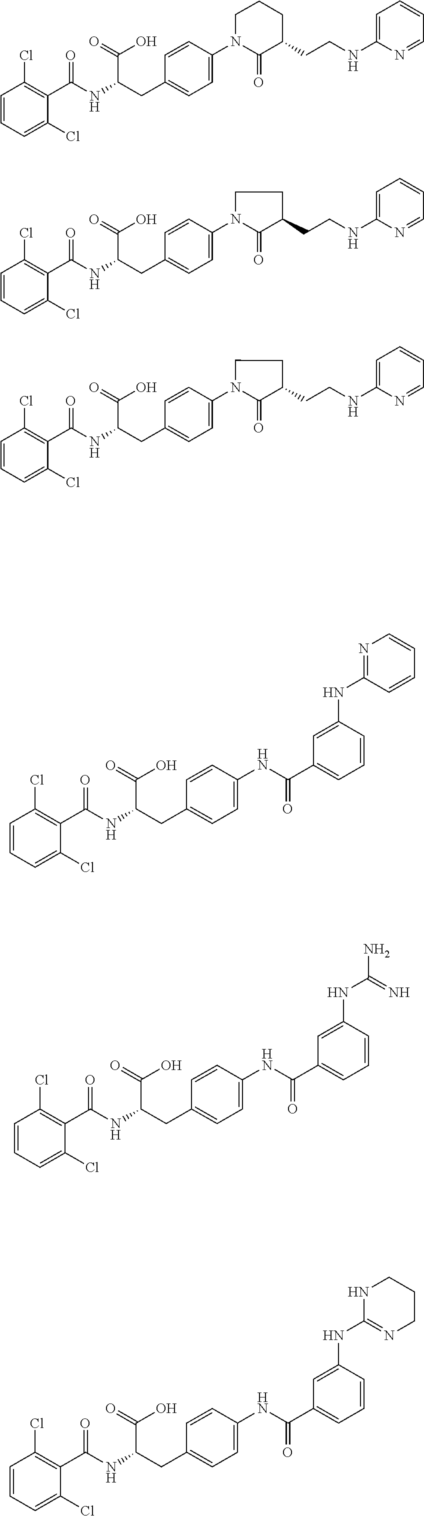 [SMILES: O=C(N[C@@H](Cc1ccc(N2CCC[C@H](CCNc3ccccn3)C2=O)cc1)C(=O)O)c1c(Cl)cccc1Cl ; O=C(N[C@@H](Cc1ccc(N2CC[C@@H](CCNc3ccccn3)C2=O)cc1)C(=O)O)c1c(Cl)cccc1Cl ; O=C(N[C@@H](Cc1ccc(N2CC[C@H](CCNc3ccccn3)C2=O)cc1)C(=O)O)c1c(Cl)cccc1Cl ; O=C(Nc1ccc(C[C@H](NC(=O)c2c(Cl)cccc2Cl)C(=O)O)cc1)c1cccc(Nc2ccccn2)c1 ; N=C(N)Nc1cccc(C(=O)Nc2ccc(C[C@H](NC(=O)c3c(Cl)cccc3Cl)C(=O)O)cc2)c1 ; O=C(Nc1ccc(C[C@H](NC(=O)c2c(Cl)cccc2Cl)C(=O)O)cc1)c1cccc(NC2=NCCCN2)c1]

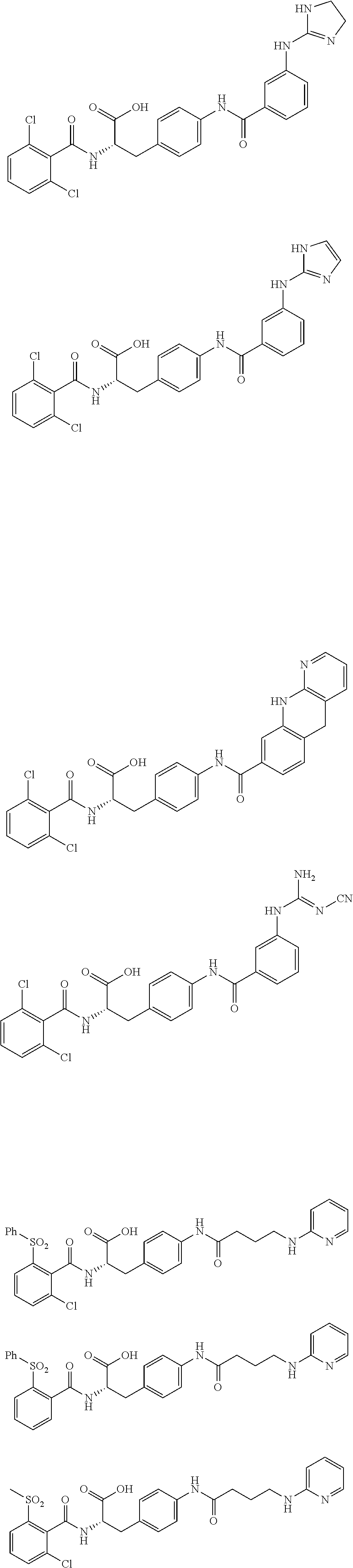

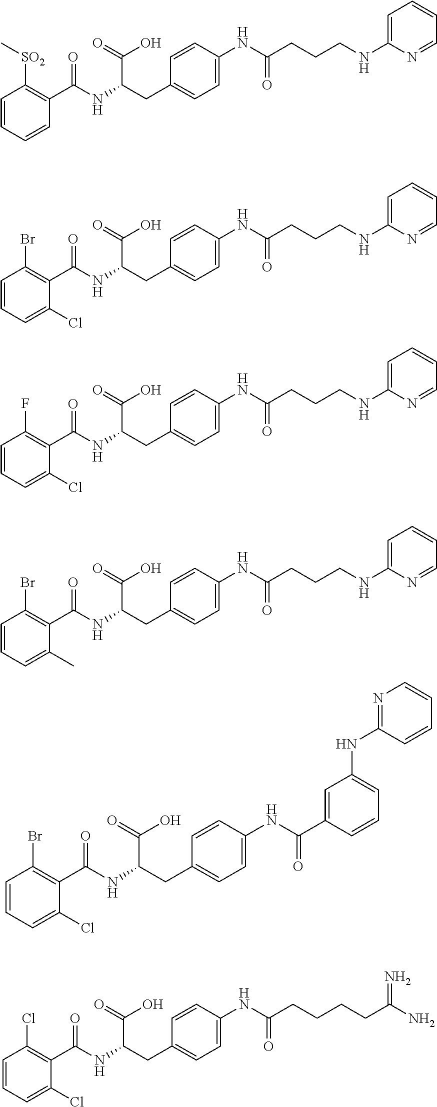 [SMILES: CS(=O)(=O)c1ccccc1C(=O)N[C@@H](Cc1ccc(NC(=O)CCCNc2ccccn2)cc1)C(=O)O ; O=C(CCCNc1ccccn1)Nc1ccc(C[C@H](NC(=O)c2c(Cl)cccc2Br)C(=O)O)cc1 ; O=C(CCCNc1ccccn1)Nc1ccc(C[C@H](NC(=O)c2c(F)cccc2Cl)C(=O)O)cc1 ; Cc1cccc(Br)c1C(=O)N[C@@H](Cc1ccc(NC(=O)CCCNc2ccccn2)cc1)C(=O)O ; O=C(Nc1ccc(C[C@H](NC(=O)c2c(Cl)cccc2Br)C(=O)O)cc1)c1cccc(Nc2ccccn2)c1 ; N=C(N)CCCCC(=O)Nc1ccc(C[C@H](NC(=O)c2c(Cl)cccc2Cl)C(=O)O)cc1]

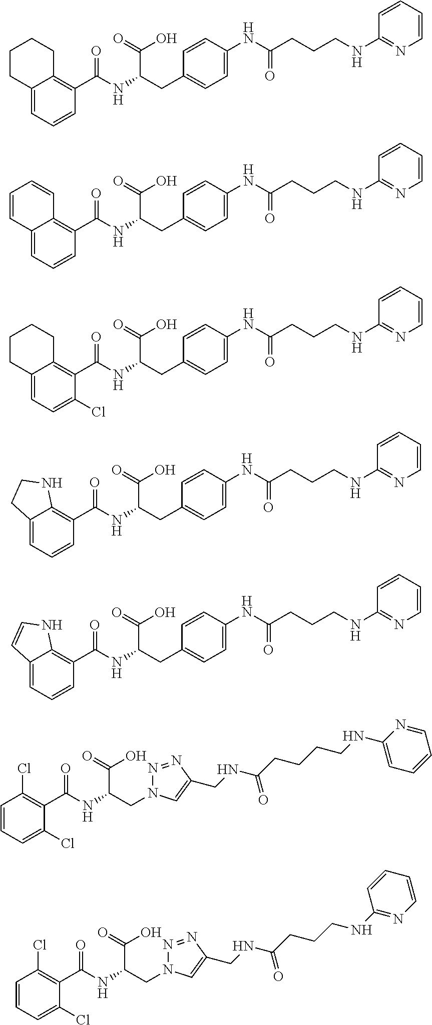

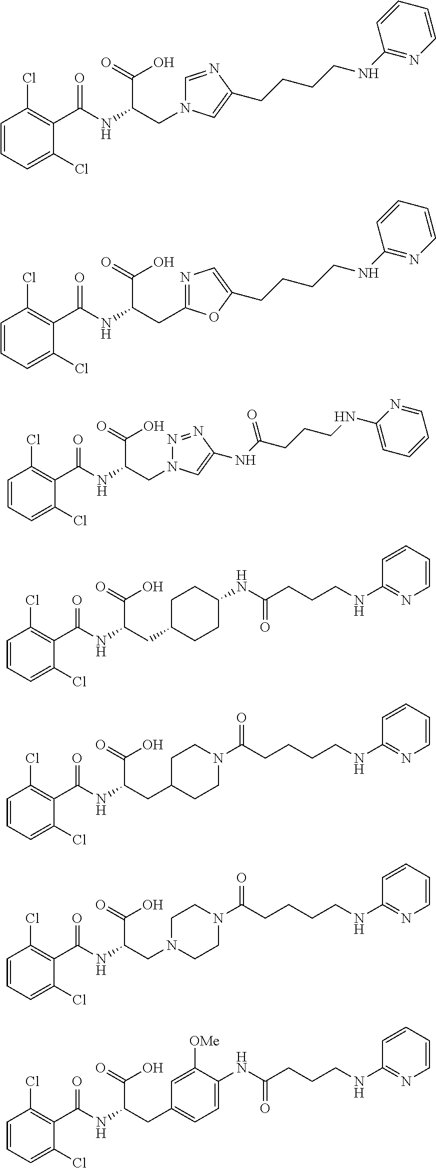

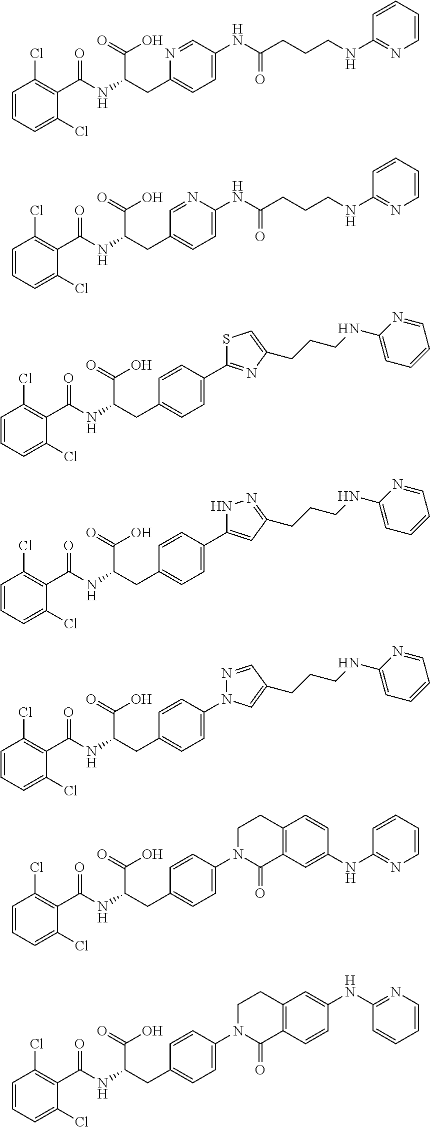

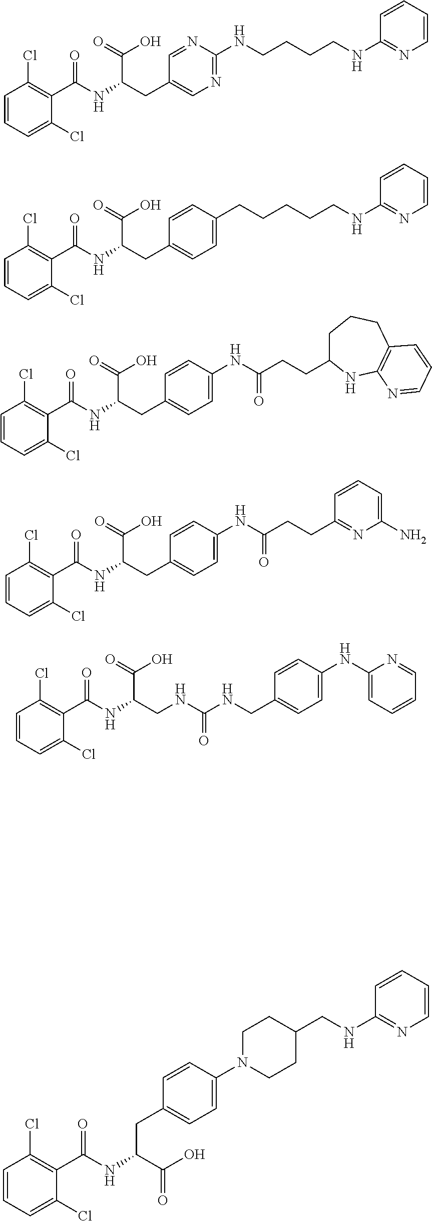 [SMILES: O=C(N[C@@H](Cc1cnc(NCCCCNc2ccccn2)nc1)C(=O)O)c1c(Cl)cccc1Cl ; O=C(N[C@@H](Cc1ccc(CCCCCNc2ccccn2)cc1)C(=O)O)c1c(Cl)cccc1Cl ; O=C(CCC1CCCc2cccnc2N1)Nc1ccc(C[C@H](NC(=O)c2c(Cl)cccc2Cl)C(=O)O)cc1 ; Nc1cccc(CCC(=O)Nc2ccc(C[C@H](NC(=O)c3c(Cl)cccc3Cl)C(=O)O)cc2)n1 ; O=C(NCc1ccc(Nc2ccccn2)cc1)NC[C@H](NC(=O)c1c(Cl)cccc1Cl)C(=O)O ; O=C(N[C@H](Cc1ccc(N2CCC(CNc3ccccn3)CC2)cc1)C(=O)O)c1c(Cl)cccc1Cl]

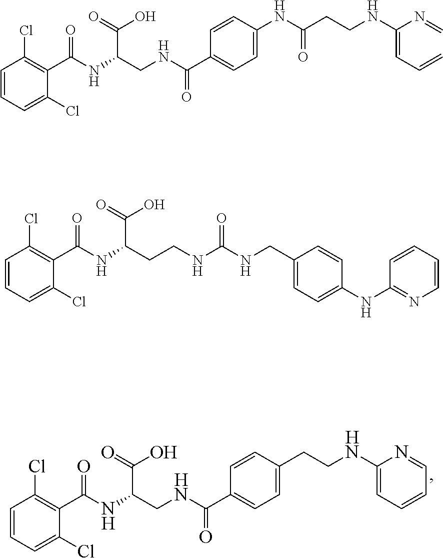 [SMILES: C=C/C=C(\N=C)NCCC(=O)Nc1ccc(C(=O)NC[C@H](NC(=O)c2c(Cl)cccc2Cl)C(=O)O)cc1 ; O=C(NCC[C@H](NC(=O)c1c(Cl)cccc1Cl)C(=O)O)NCc1ccc(Nc2ccccn2)cc1 ; O=C(NC[C@H](NC(=O)c1c(Cl)cccc1Cl)C(=O)O)c1ccc(CCNc2ccccn2)cc1]

or a pharmaceutically acceptable salt thereof. See U.S. Pat. No. 10,214,522, which is hereby incorporated by reference it its entirety.

In some embodiments, the integrin antagonist has the structure

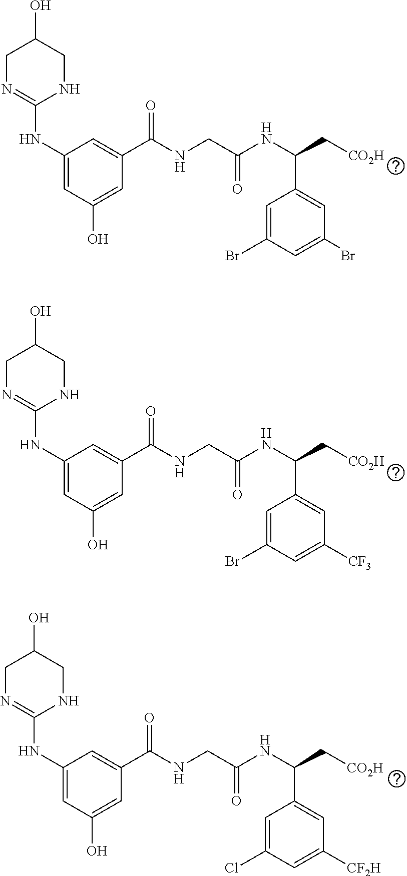 [SMILES: O=C(O)C[C@H](NC(=O)CNC(=O)c1cc(O)cc(NC2=NCC(O)CN2)c1)c1cc(Br)cc(Br)c1 ; O=C(O)C[C@H](NC(=O)CNC(=O)c1cc(O)cc(NC2=NCC(O)CN2)c1)c1cc(Br)cc(C(F)(F)F)c1 ; O=C(O)C[C@H](NC(=O)CNC(=O)c1cc(O)cc(NC2=NCC(O)CN2)c1)c1cc(Cl)cc(C(F)F)c1]

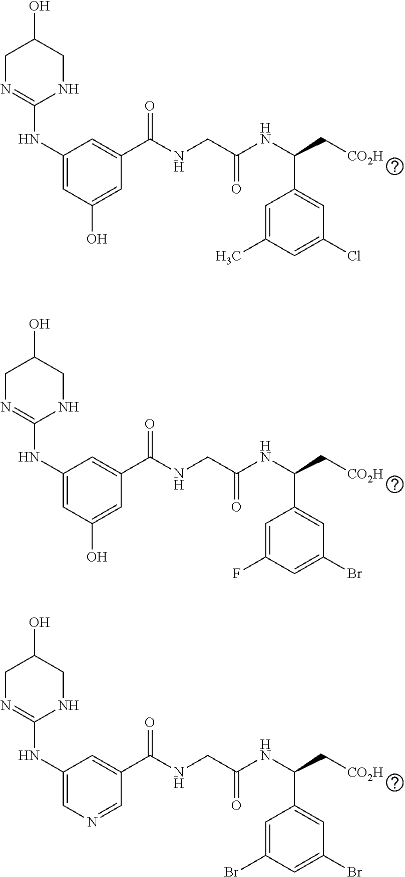 [SMILES: Cc1cc(Cl)cc([C@H](CC(=O)O)NC(=O)CNC(=O)c2cc(O)cc(NC3=NCC(O)CN3)c2)c1 ; O=C(O)C[C@H](NC(=O)CNC(=O)c1cc(O)cc(NC2=NCC(O)CN2)c1)c1cc(F)cc(Br)c1 ; O=C(O)C[C@H](NC(=O)CNC(=O)c1cncc(NC2=NCC(O)CN2)c1)c1cc(Br)cc(Br)c1]

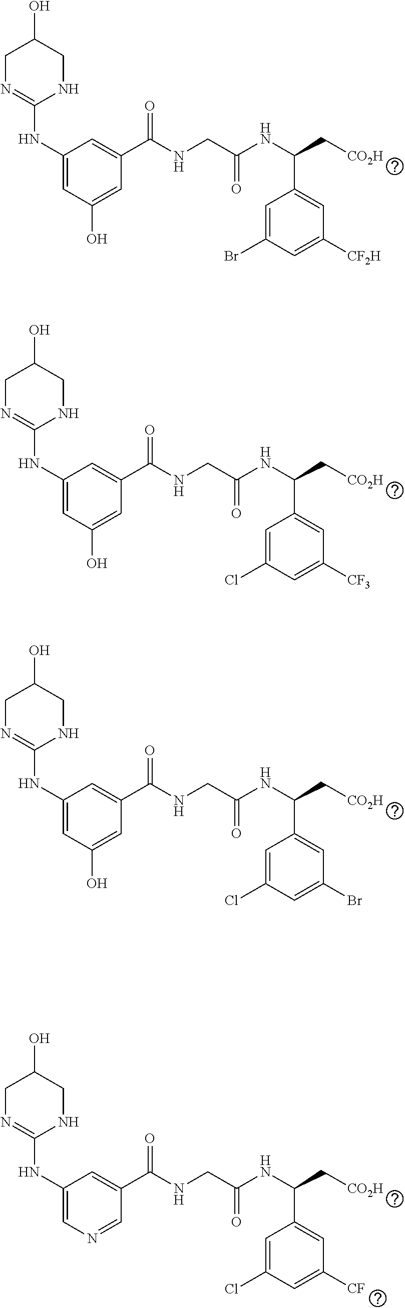

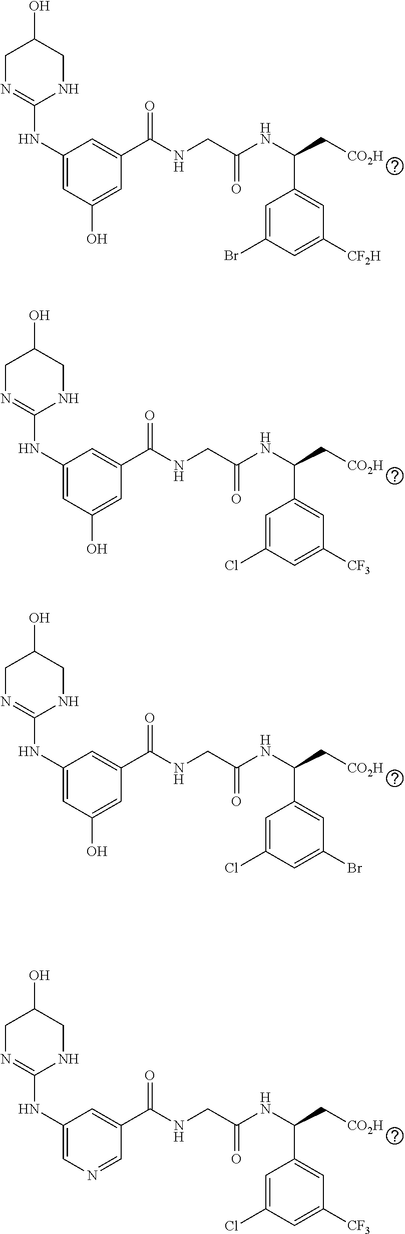 [SMILES: O=C(O)C[C@H](NC(=O)CNC(=O)c1cc(O)cc(NC2=NCC(O)CN2)c1)c1cc(Br)cc(C(F)F)c1 ; O=C(O)C[C@H](NC(=O)CNC(=O)c1cc(O)cc(NC2=NCC(O)CN2)c1)c1cc(Cl)cc(C(F)(F)F)c1 ; O=C(O)C[C@H](NC(=O)CNC(=O)c1cc(O)cc(NC2=NCC(O)CN2)c1)c1cc(Cl)cc(Br)c1 ; O=C(O)C[C@H](NC(=O)CNC(=O)c1cncc(NC2=NCC(O)CN2)c1)c1cc(Cl)cc(C(F)(F)F)c1]

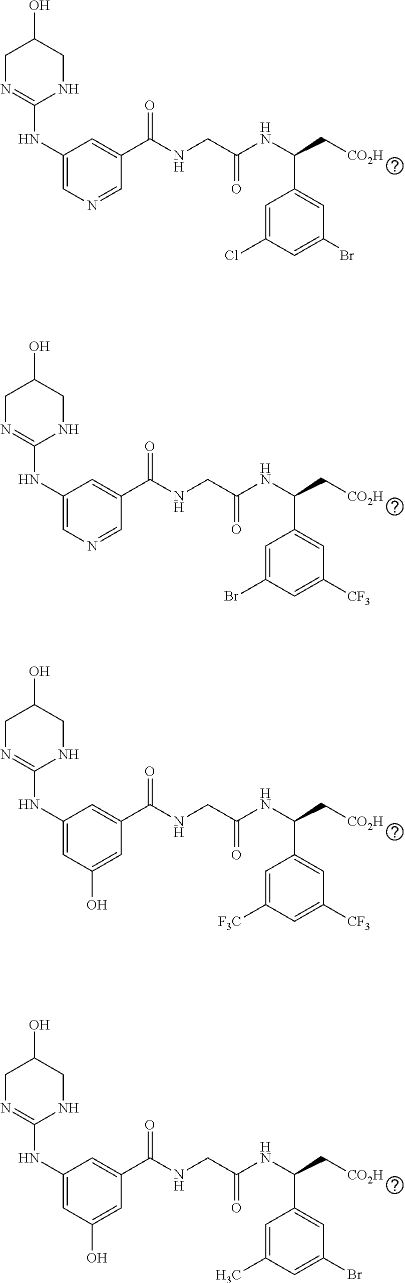 [SMILES: O=C(O)C[C@H](NC(=O)CNC(=O)c1cncc(NC2=NCC(O)CN2)c1)c1cc(Cl)cc(Br)c1 ; O=C(O)C[C@H](NC(=O)CNC(=O)c1cncc(NC2=NCC(O)CN2)c1)c1cc(Br)cc(C(F)(F)F)c1 ; O=C(O)C[C@H](NC(=O)CNC(=O)c1cc(O)cc(NC2=NCC(O)CN2)c1)c1cc(C(F)(F)F)cc(C(F)(F)F)c1 ; Cc1cc(Br)cc([C@H](CC(=O)O)NC(=O)CNC(=O)c2cc(O)cc(NC3=NCC(O)CN3)c2)c1]

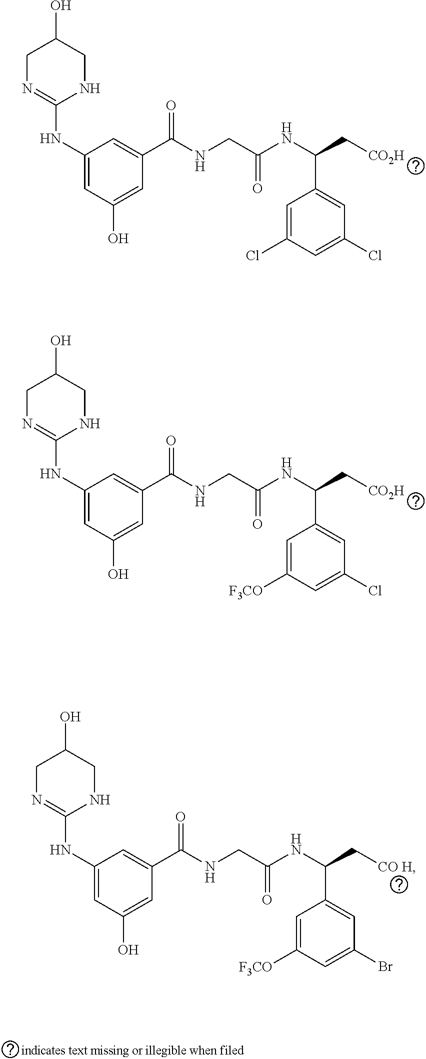

or a pharmaceutically acceptable salt thereof. See US 2018/0072684, which is hereby incorporated by reference it its entirety.

In some embodiments, the non-steroidal Farnesoid X receptor (FXR) agonist is cilofexor.

In some embodiments, the combinations of the invention comprise selonsertib, firsocostat or cilofexor. In some embodiments, the combinations of the invention comprise selonsertib and firsocostat. In some embodiments, the combinations of the invention comprises selonsertib and cilofexor. In some embodiments, the combinations of the invention comprises fircosostat and cilofexor. In some embodiments, the combinations of the invention comprise selonsertib, firsocostat and cilofexor.

In some embodiments, the pharmaceutically acceptable carrier or vehicle includes, but is not limited to, a binder, filler, diluent, disintegrant, wetting agent, lubricant, glidant, coloring agent, dye-migration inhibitor, sweetening agent or flavoring agent.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof.

Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. In some embodiments, the binder is hydroxypropylcellulose.

The binder or filler can be present from about 2% to about 49% by weight of the compositions of the invention provided herein or any range within these values. In some embodiments, the binder or filler is present in the composition of the invention from about 5% to about 15% by weight. In some embodiments, the binder or filler is present in the composition of the invention at about 5%, about 6%, about 7%, about 8%, about 9%, about 8%, about 10%, about 11%, about 12%, about 13%, about 14%, or about 15% by weight or any range within any of these values.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. In some embodiments, the diluent is lactose monohydrate. In some embodiments, the diluent is lactose monohydrate Fast-Flo 316 NF.

The compositions of the invention can comprise a diluent, e.g., from about 5% to about 49% of a diluent by weight of composition or any range between any of these values. In some embodiments, the diluent is present in the compositions of the invention from about 15% to about 30% by weight. In some embodiments, the diluent is present in the composition of the invention at about 15%, about 16%, about 17%, about 18%, about 19%, about 18%, about 20%, about 21%, about 22%, about 23%, about 24%, about 25%, about 26%, about 27%, about 28%, about 29%, or about 30% by weight or any range within any of these values.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the compositions of the invention can vary. In some embodiments, the disintegrant is croscarmellose sodium. In some embodiments, the disintegrant is croscarmellose sodium NF (Ac-Di-Sol).

The compositions of the invention can comprise a disintegrant, e.g., from about 0.5% to about 15% or from about 1% to about 10% by weight of a disintegrant. In some embodiments, the compositions of the invention comprise a disintegrant in an amount of about 5%, about 6%, about 7%, about 8%, about 9%, about 8%, about 10%, about 11%, about 12%, about 13%, about 14%, or about 15% by weight of the composition or in any range within any of these values.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. In some embodiments, the lubricant is magnesium stearate.

The compositions of the invention can comprise a lubricant, e.g., about 0.1 to about 5% by weight of a lubricant. In some embodiments, the compositions of the invention comprise a lubricant in an amount of about 0.5%, 0.6%, 0.7%, 0.8%, 0.9%, 0.8%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%, by weight of the composition or in any range within any of these values.

Suitable glidants include colloidal silicon dioxide, CAB-O-SIL® (Cabot Co. of Boston, Mass.), and talc, including asbestos-free talc.

Coloring agents include any of the approved, certified, water soluble FD&C dyes, and water insoluble FD&C dyes suspended on alumina hydrate, and color lakes and mixtures thereof.

Flavoring agents include natural flavors extracted from plants, such as fruits, and synthetic blends of compounds that provide a pleasant taste sensation, such as peppermint and methyl salicylate.

Sweetening agents include sucrose, lactose, mannitol, syrups, glycerin, sucralose, and artificial sweeteners, such as saccharin, stevioside (Stevia) and aspartame.

Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether.

Solvents include glycerin, sorbitol, ethyl alcohol, and syrup.

Examples of non-aqueous liquids utilized in emulsions include mineral oil and cottonseed oil. Organic acids include citric and tartaric acid. Sources of carbon dioxide include sodium bicarbonate and sodium carbonate.

The compounds of the invention and the compositions of the invention can be formulated for administration by a variety of means including orally, parenterally, by inhalation spray, topically, or rectally in formulations containing pharmaceutically acceptable carriers, adjuvants and vehicles. The term “parenteral” as used here includes subcutaneous, intravenous, intramuscular, and intraarterial injections with a variety of infusion techniques. Intraarterial and intravenous injection as used herein includes administration through catheters.

The compounds of the invention and the compositions of the invention can be formulated in accordance with the routine procedures adapted for desired administration route. Accordingly, the compositions of the invention can take such forms as suspensions, solutions or emulsions in oily or aqueous vehicles, and can contain formulatory agents such as suspending, stabilizing and/or dispersing agents. The compounds of the invention and the compositions of the invention can be formulated as a preparation suitable for implantation or injection. Thus, for example, the compositions of the invention can be formulated with suitable polymeric or hydrophobic materials (e.g., as an emulsion in an acceptable oil) or ion exchange resins, or as sparingly soluble derivatives (e.g., as a sparingly soluble salt). The compounds of the invention and the compositions of the invention can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In some embodiments, the compositions of the invention are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some embodiments, compositions of the invention suitable for oral administration are in the form of a tablet or a capsule. In some embodiments, the composition of the invention is in a form of a tablet. In some embodiments, the composition of the invention is in a form of a capsule. In some embodiments, the compound of the invention is contained in a capsule.

In some embodiments, capsules are immediate release capsules. Non-limiting example of a capsule is a Coni-snap® hard gelatin capsule.

The compositions of the invention can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some embodiments, the coating is a film coating. In some embodiments, the film coating comprises Opadry White and simethicone emulsion 30% USP.

In some embodiments, the compound of the invention is contained in a tablet. In some embodiments, the compound of the invention is contained in a compressed tablet. In some embodiments, the compound of the invention is contained in a film-coated compressed tablet. In some embodiments, the compositions of the invention are in the form of film-coated compressed tablets.

In some embodiments, the compositions of the invention is prepared by fluid bed granulation of the compound of the invention with one or more pharmaceutically acceptable carrier, vehicle, or excipients. In some embodiments, the compositions of the invention prepared by fluid bed granulation process can provide tablet formulation with good flowability, good compressibility, fast dissolution, good stability, and/or minimal to no cracking. In some embodiments, the fluid bed granulation process allows preparation of formulations having high drug loading, such as over 70% or over 75% of a compound of the invention.

The compositions of the invention can be in the form of soft or hard capsules, which can be made from gelatin, methylcellulose, starch, or calcium alginate. The hard gelatin capsule, also known as the dry-filled capsule (DFC), can comprise of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells can contain a preservative to prevent the growth of microorganisms. Suitable preservatives are those as described herein, including methyl- and propyl-parabens, and sorbic acid. The liquid, semisolid, and solid dosage forms provided herein can be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules can also be coated as known by those of skill in the art in order to modify or sustain dissolution of the active ingredient.

The compositions of the invention can be in liquid or semisolid dosage forms, including emulsions, solutions, suspensions, elixirs, and syrups. An emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

The compositions of the invention for oral administration can be also provided in the forms of liposomes, micelles, microspheres, or nanosystems. Miccellar dosage forms can be prepared as described in U.S. Pat. No. 6,350,458.

The compositions of the invention can be provided as non-effervescent or effervescent, granules and powders, to be reconstituted into a liquid dosage form. Pharmaceutically acceptable carriers and excipients used in the non-effervescent granules or powders can include diluents, sweeteners, and wetting agents. Pharmaceutically acceptable carriers and excipients used in the effervescent granules or powders can include organic acids and a source of carbon dioxide.

Coloring and flavoring agents can be used in all of the above dosage forms. And, flavoring and sweetening agents are especially useful in the formation of chewable tablets and lozenges.

The compositions of the invention can be formulated as immediate or modified release dosage forms, including delayed-, extended, pulsed-, controlled, targeted-, and programmed-release forms.

In some embodiments, the compositions of the invention comprise a film-coating.

The compositions of the invention can comprise another active ingredient that does not impair the composition's therapeutic or prophylactic efficacy or can comprise a substance that augments or supplements the composition's efficacy.

The tablet dosage forms can comprise the compound of the invention in powdered, crystalline, or granular form, and can further comprise a carrier or vehicle described herein, including binder, disintegrant, controlled-release polymer, lubricant, diluent, or colorant.

In some embodiments, the compositions of the invention can further comprise an excipient such as a diluent, a disintegrant, a wetting agent, a binder, a glidant, a lubricant, or any combination thereof. In some embodiments, a tablet comprises a binder. And, in some embodiments, the binder comprises microcrystalline cellulose, dibasic calcium phosphate, sucrose, corn starch, polyvinylpyrridone, hydroxypropyl cellulose, hydroxymethyl cellulose, or any combination thereof. In other embodiments, the tablet comprises a disintegrant. In other embodiments, the disintegrant comprises sodium croscarmellose, sodium starch glycolate, or any combination thereof. In other embodiments, the tablet comprises a lubricant. And, in some embodiments, the lubricant comprises magnesium stearate stearic acid, hydrogenated oil, sodium stearyl fumarate, or any combination thereof.

In some embodiments, the compositions of the invention are in the form of a tablet that comprises a binder such as any of the binders described herein.

In some embodiments, the compositions of the invention are in the form of a tablet that comprises a disintegrant such as any of the disintegrants described herein.

In some embodiments, the compositions of the invention are in the form of a tablet that comprises a lubricant such as any of the lubricants described herein.

In some embodiments, the compositions of the invention can be in a modified release or a controlled release dosage form. In some embodiments, the compositions of the invention can comprise particles exhibiting a particular release profile. For example, the composition of the invention can comprise a compound of the invention in an immediate release form while also comprising a statin or a pharmaceutically acceptable salt, solvate, ester, amide, or prodrug thereof in a modified release form, both compressed into a single tablet. Other combination and modification of release profile can be achieved as understood by one skilled in the art. Examples of modified release dosage forms suited for pharmaceutical compositions of the instant invention are described, without limitation, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

In some embodiments, the compositions of the invention are a matrix-controlled release dosage form. In some embodiments, the release profile of the compound of the invention and of the other pharmaceutically active agent is the same or different. Suitable matrix-controlled release dosage forms are described, for example, in Takada et al in “Encyclopedia of Controlled Drug Delivery,” Vol. 2, Mathiowitz ed., Wiley, 1999.

In some embodiments, the matrix-controlled release form comprises an erodible matrix comprising water-swellable, erodible, or soluble polymers, including synthetic polymers, and naturally occurring polymers and derivatives, such as polysaccharides and proteins.

In some embodiments, the erodible matrix of the matrix-controlled release form comprises chitin, chitosan, dextran, or pullulan; gum agar, gum arabic, gum karaya, locust bean gum, gum tragacanth, carrageenans, gum ghatti, guar gum, xanthan gum, or scleroglucan; starches, such as dextrin or maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), carrrboxymethyl ethyl cellulose (CMEC,) hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate phthalate (CAP), cellulose acetate trimellitate (CAT), hydroxypropyl methyl cellulose (HPMC), HPMCP, HPMCAS, hydroxypropyl methyl cellulose acetate trimellitate (HPMCAT), or ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; or other acrylic acid derivatives, such as homopolymers and copolymers of butylmethacrylate, methylmethacrylate, ethylmethacrylate, ethylacrylate, (2-dimethylaminoethyl)methacrylate, or (trimethylaminoethyl)methacrylate chloride; or any combination thereof.

In other embodiments, the compositions of the invention are in a matrix-controlled modified release form comprising a non-erodible matrix. In some embodiments, the statin, the compound of the invention is dissolved or dispersed in an inert matrix and is released primarily by diffusion through the inert matrix once administered. In some embodiments, the non-erodible matrix of the matrix-controlled release form comprises an insoluble polymer, such as polyethylene, polypropylene, polyisoprene, polyisobutylene, polybutadiene, polymethylmethacrylate, polybutylmethacrylate, chlorinated polyethylene, polyvinylchloride, a methyl acrylate-methyl methacrylate copolymer, an ethylene-vinylacetate copolymer, an ethylene/propylene copolymer, an ethylene/ethyl acrylate copolymer, a vinylchloride copolymer with vinyl acetate, a vinylidene chloride, an ethylene or a propylene, an ionomer polyethylene terephthalate, a butyl rubber epichlorohydrin rubber, an ethylene/vinyl alcohol copolymer, an ethylene/vinyl acetate/vinyl alcohol terpolymer, an ethylene/vinyloxyethanol copolymer, a polyvinyl chloride, a plasticized nylon, a plasticized polyethyleneterephthalate, a natural rubber, a silicone rubber, a polydimethylsiloxane, a silicone carbonate copolymer, or a hydrophilic polymer, such as an ethyl cellulose, a cellulose acetate, a crospovidone, or a cross-linked partially hydrolyzed polyvinyl acetate; a fatty compound, such as a carnauba wax, a microcrystalline wax, or a triglyceride; or any combination thereof.

The compositions of the invention that are in a modified release dosage form can be prepared by methods known to those skilled in the art, including direct compression, dry or wet granulation followed by compression, melt-granulation followed by compression.

In some embodiments, the compositions of the invention comprise a tablets-in-capsule system, which can be a multifunctional and multiple unit system comprising versatile mini-tablets in a hard gelatin capsule. The mini-tablets can be rapid-release, extended-release, pulsatile, delayed-onset extended-release minitablets, or any combination thereof. In some embodiments, combinations of mini-tablets or combinations of mini-tablets and minibeads comprising multiple active pharmaceutical agents can each have specific lag times, of release multiplied pulsatile drug delivery system (DDS), site-specific DDS, slow-quick DDS, quick/slow DDS and zero-order DDS.

In some embodiments, the compositions of the invention are in an osmotic-controlled release dosage form.

In some embodiments, the osmotic-controlled release device comprises a one-chamber system, a two-chamber system, asymmetric membrane technology (AMT), an extruding core system (ECS), or any combination thereof. In some embodiments, such devices comprise at least two components: (a) the core which contains the active pharmaceutical agent(s); and (b) a semipermeable membrane with at least one delivery port, which encapsulates the core. The semipermeable membrane controls the influx of water to the core from an aqueous environment of use so as to cause drug release by extrusion through the delivery port(s).

In some embodiments, the core of the osmotic device optionally comprises an osmotic agent, which creates a driving force for transport of water from the environment of use into the core of the device. One class of osmotic agents useful in the compositions of invention comprises water-swellable hydrophilic polymers, which are also referred to as “osmopolymers” or “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), cross-linked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

Another class of osmotic agents useful in the compositions of the invention comprises osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the compound of the invention dissolves following administration. For example, an amorphous sugar, such as Mannogeme EZ (SPI Pharma, Lewes, Del.) can be included to provide faster delivery during the first couple of hours (e.g., about 1 to about 5 hrs) to promptly produce prophylactic or therapeutic efficacy, and gradually and continually release of the remaining amount to maintain the desired level of therapeutic or prophylactic effect over an extended period of time. In some embodiments, the compound of the invention is released from the compositions of the invention at such a rate to replace the amount of the compound of the invention metabolized or excreted by the subject.

The core can also include a wide variety of other excipients and carriers as described herein to enhance the performance of the dosage form or to promote stability or processing.

Materials useful for forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

The semipermeable membranes can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the compound of the invention released and the release rate can substantially be modulated via the thickness and porosity of the semipermeable membrane, the composition of the core, and the number, size, and position of the delivery ports.

In some embodiments, the pharmaceutical composition in an osmotic controlled-release dosage form can further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical composition provided herein is formulated as asymmetric membrane technology (AMT) controlled-release dosage form that comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In some embodiments, the pharmaceutical composition provided herein is formulated as ESC controlled-release dosage form that comprises an osmotic membrane that coats a core comprising the compound of the invention, hydroxylethyl cellulose, and other pharmaceutically acceptable excipients.

In some embodiments, the compositions of the invention are a modified release dosage form that is fabricated as a multiparticulate-controlled release dosage form that comprises a plurality of particles, granules, or pellets, microparticulates, beads, microcapsules and microtablets, ranging from about 10 μm to about 3 mm, about 50 μm to about 2.5 mm, or from about 100 μm to 1 mm in diameter.

The multiparticulate-controlled release dosage forms can provide a prolonged release dosage form with an improved bioavailability. Suitable carriers to sustain the release rate of the compound of the invention include, without limitation, ethyl cellulose, HPMC, HPMC-phtalate, colloidal silicondioxide and Eudragit-RSPM.

Compositions of the invention in pellet form can comprise 50-80% (w/w) of a drug and 20-50% (w/w) of microcrystalline cellulose or other polymers. Suitable polymers include, but are not limited to, microcrystalline wax, pregelatinized starch and maltose dextrin.

Beads can be prepared in capsule and tablet dosage forms. Beads in tablet dosage form can demonstrate a slower dissolution profile than microparticles in capsule form. Microparticle fillers suitable for compositions and therapeutic or prophylactic methods of the invention include, without limitation, sorbitan monooleate (Span 80), HPMC, or any combination thereof. Suitable dispersions for controlled release latex include, for example, ethyl-acrylate and methyl-acrylate.

In some embodiments, the compositions of the invention are in the form or microcapsules and/or microtablets. In some embodiments, microcapsules comprise extended release polymer microcapsules containing a statin and a compound of the invention with various solubility characteristics. Extended release polymer microcapsules can be prepared with colloidal polymer dispersion in an aqueous environment. In other embodiments, microcapsules suitable for the compositions and methods provided herein can be prepared using conventional microencapsulating techniques (Bodmeier & Wang, 1993).

Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989. Excipients for such technologies are commercially available and described in US Pharmacopeia.

Other excipients as described herein can be blended with the compositions of the invention to aid in processing and forming the multiparticulates. The resulting particles can themselves constitute the multiparticulate dosage form or can be coated by various film-forming materials, such as enteric polymers, water-swellable, or water-soluble polymers. The multiparticulates can be further processed as a capsule or a tablet.

In other embodiments, the compositions of the invention are in a dosage form that has an instant releasing component and at least one delayed releasing component, and is capable of giving a discontinuous release of the compound in the form of at least two consecutive pulses separated in time from 0.1 hr to 24 hrs.

In some embodiments, the compositions of the invention comprise from about 1 mg to about 1000 mg of a compound of the invention or any amount ranging from and to these values. In some embodiments, the compositions of the invention comprise from about 1 mg to about 500 mg of a compound of the invention or any amount ranging from and to these values. In some embodiments, the compositions of the invention comprise from about 1 mg to about 400 mg of a compound of the invention or any amount ranging from and to these values.

In other embodiments, the compositions of the invention comprise a compound of the invention in an amount that is a molar equivalent to about 1 mg to about 1000 mg of a compound of the invention or any amount ranging from and to these values. In other embodiments, the compositions of the invention comprise a compound of the invention in an amount that is a molar equivalent to about 1 mg to about 500 mg of a compound of the invention or any amount ranging from and to these values. In other embodiments, the compositions of the invention comprise a compound of the invention in an amount that is a molar equivalent to about 1 mg to about 400 mg of a compound of the invention or any amount ranging from and to these values.

In some embodiments, the compositions of the invention comprise a compound of the invention in an amount of about 10 wt % to about 99 wt % of the total weight of the composition of the invention.

### Methods of the Invention

The present invention provides methods for treating or preventing a liver disorder, dyslipidemia, dyslipoproteinemia, a renal disease, a disorder of glucose metabolism, a disorder of lipid metabolism, a disorder of glucid metabolism, a cardiovascular disease, a vascular disease, a metabolic syndrome, a complication associated with metabolic syndrome, a PPAR-associated disorder, septicemia, a thrombotic disorder, obesity, diabetic nephropathy, diabetic retinopathy, atherosclerosis, pancreatitis, a cerebrovascular disease, a disorder related to neovascularization, hypertension, cancer, inflammation, an inflammatory disease, a neurodegenerative disease, an autoimmune disease, a neoplastic disease, muscle atrophy, cholestasis, mitochondrial dysfunction, an ocular disease, a lysosomal storage disease, or impotence, comprising administering to a subject in need thereof an effective amount of a compound of the invention or a composition of the invention. The present invention provides methods for treating or preventing a kidney disease, comprising administering to a subject in need thereof an effective amount of a compound of the invention or a composition of the invention.

In some embodiments, the present invention provides methods for treating or preventing a liver disorder, comprising administering to a subject in need thereof an effective amount of a compound of the invention or a composition of the invention. In some embodiments, the liver disorder involves pathological disruption, inflammation, degeneration, apoptosis, or proliferation of liver cells. In some embodiments, the liver disorder is liver fibrosis, fatty liver disease, non-alcoholic fatty liver disease (NAFLD) or non-alcoholic steatohepatitis (NASH).

In some embodiments, the present invention provides methods for reducing an abnormally high concentration of alanine transaminase (ALT), aspartate transaminase (AST), alkaline phosphatase (ALP), bilirubin, gamma-glutamyltransferase (GGT), L-lactate dehydrogenase (LD), prothrombin time (PT), creatinine, or total protein in a subject's blood plasma or blood serum, comprising administering to a subject in need thereof an effective amount of a compound of the invention or a composition of the invention. In some embodiments, the present invention provides methods for elevating an abnormally low concentration of albumin or total protein, in a subject's blood plasma or blood serum, comprising administering to a subject in need thereof an effective amount of a compound of the invention or a composition of the invention.

An “abnormally high concentration” of ALT in a subject's blood plasma or blood serum is greater than 56 units/liter. In some embodiments, the reducing is to a normal concentration. In some embodiments, the normal concentration of ALT in a subject's blood plasma or blood serum ranges from about 7 units/liter to about 56 units/liter.

An “abnormally high concentration” of AST in a subject's blood plasma or blood serum is greater than 48 units/liter. In some embodiments, the reducing is to a normal concentration. In some embodiments, the normal concentration of AST in a subject's blood plasma or blood serum ranges from about 8 units/liter to about 48 units/liter.

An “abnormally high concentration” of ALP in a subject's blood plasma or blood serum is greater than 129 units/liter. In some embodiments, the reducing is to a normal concentration. In some embodiments, the normal concentration of ALP in a subject's blood plasma or blood serum ranges from about 40 units/liter to about 129 units/liter.

An “abnormally low concentration” of albumin in a subject's blood plasma or blood serum is less than 3.5 g/dL. In some embodiments, the elevating is to a normal concentration. In some embodiments, the normal concentration of albumin in a subject's blood plasma or blood serum ranges from about 3.5 g/dL to about 5.0 g/dL.

An “abnormally high concentration” of bilirubin in a subject's blood plasma or blood serum is greater than 1.2 mg/dL. In some embodiments, the reducing is to a normal concentration. In some embodiments, the normal concentration of bilirubin in a subject's blood plasma or blood serum ranges from about 0.1 mg/dL to about 1.2 mg/dL.

An “abnormally high concentration” of GGT in a subject's blood plasma or blood serum is greater than 61 units/liter. In some embodiments, the reducing is to a normal concentration. In some embodiments, the normal concentration of GGT in a subject's blood plasma or blood serum ranges from about 8 units/liter to about 61 units/liters.

An “abnormally high concentration” of LD in a subject's blood plasma or blood serum is greater than 222 units/liter. In some embodiments, the reducing is to a normal concentration. In some embodiments, the normal concentration of LD in a subject's blood plasma or blood serum ranges from about 122 units/liter to about 222 units/liters.

An “abnormally high concentration” of PT in a subject's blood plasma or blood serum is greater than 12.5 seconds. In some embodiments, the reducing is to a normal concentration. In some embodiments, the normal concentration of PT in a subject's blood plasma or blood serum ranges from about 9.4 seconds to about 12.5 seconds.

An “abnormally high concentration” of creatinine in a subject's blood plasma or blood serum is greater than 1.5 mg/dL, which corresponds to a glomerular filtration rate (GFR) of approximately 30 mL/min and indicative of renal failure. In some embodiments, the reducing is to a normal concentration. In some embodiments, the normal concentration of creatinine in a subject's blood plasma or blood serum ranges from about 0.84 mg/dL to about 1.21 mg/dL. (about 74.3 μmol/L to about 107 μmol/L).

An “abnormally high concentration” of total protein in a subject's blood plasma or blood serum is greater than 7.9 g/dL. An “abnormally low concentration” of total protein in a subject's blood plasma or blood serum is less than 6.3 g/dL. In some embodiments, the reducing is to a normal concentration. In some embodiments, the elevating is to a normal concentration. In some embodiments, the normal concentration of total protein in a subject's blood plasma or blood serum ranges from about 6.3 g/dL to about 7.9 g/dL.

In some embodiments, the present invention provides methods for treating or preventing NAFLD or NASH, comprising administering to a subject in need thereof an effective amount of a compound of the invention or the composition of the invention.

In some embodiments, the present invention provides methods for treating or preventing dyslipidemia, comprising administering to a subject in need thereof an effective amount of the compound of the invention or the composition of the invention. In some embodiments, the dyslipidemia is hyperlipidemia or an abnormally low concentration of high density lipoprotein cholesterol (HDL-C) in the subject's blood plasma or blood serum. The term “dyslipidemia” refers to a disorder that leads to or is manifested by an aberrant level of circulating lipids.

In some embodiments, the present invention provides methods for restoring blood plasma or blood serum concentration of total-cholesterol, low density lipoprotein cholesterol (LDL-C), HDL-C, non-HDL-C or free triglycerides to a normal or recommended concentration or ratio. Accordingly, to the extent that levels of lipids in the blood plasma or blood serum are abnormally high, the compounds of the invention or the compositions of the invention can be administered to a patient to restore normal levels. Normal levels of lipids are well known to those skilled in the art. For example, normal blood levels of total-cholesterol, low density lipoprotein cholesterol (LDL-C), HDL-C, non-HDL-C, free triglycerides and others parameters relating to lipid metabolism can be found at the web site of the American Heart Association, The National Lipid Association and that of the National Cholesterol Education Program of the National Heart, Lung and Blood Institute. In some embodiments, a recommended concentration of HDL-C in the blood plasma or the blood serum is above 35 mg/dl. In some embodiments, a recommended concentration of LDL-C in the blood plasma or the blood serum is below 100 mg/dl. In some embodiments, a recommended LDL-C:HDL-C ratio in the blood plasma or in the blood serum is below 5:1, in some embodiments, 3.5:1. In some embodiments, a recommended concentration of free triglycerides in the blood plasma or the blood serum is less than 200 mg/dl.

In some embodiments, the present invention provides methods for treating or preventing hyperlipidemia, comprising administering to a subject in need thereof an effective amount of a compound of the invention or a composition of the invention. In some embodiments, hyperlipidemia is hypercholesterolemia, familial hypercholesterolemia, hypertriglyceridemia, or familial combined hyperlipidemia. In some embodiments, hyperlipidemia is characterized by an abnormally reduced or deficient lipoprotein lipase level or activity in the subject's blood plasma or blood serum, or an abnormally high concentration of ketone bodies, lipoprotein(a) cholesterol (Lp(a)-C), low density lipoprotein (LDL), very low density lipoproteins cholesterol (VLDL-C) or non-esterified fatty acids (NEFA) in the subject's blood plasma or blood serum. In some embodiments, the reduced or deficient lipoprotein lipase level or activity is a result of a lipoprotein lipase mutation. In some embodiments, the reduced or deficient lipoprotein lipase level or activity is a result of a mutation in a gene encoding a lipoprotein lipase.

Non-limiting examples of ketone bodies include acetoacetate, beta-hydroxybutyrate, and acetone. An “abnormally high concentration” of ketone bodies in a subject's blood plasma or blood serum is 1 mg/dL or greater (<0.1 mmol/L). In some embodiments, the present invention provides methods for reducing an abnormally high concentration of ketone bodies in a subject's blood plasma or blood serum, wherein the concentration is 1 mg/dL or greater. In some embodiments, the reducing is to a normal level. In some embodiments, the normal level is less than 1 mg/dL (<0.1 mmol/L). See Devkota, B. P. et al. Medscape emedicine, updated Oct. 30, 2015.

An “abnormally high concentration” of VLDL-C in a subject's blood plasma or blood serum is greater than 30 mg/dL (1.7 mmol/L). In some embodiments, the present invention provides methods for reducing VLDL-C concentration in a subject's blood plasma or blood serum, wherein the VLDL-C concentration is greater than 30 mg/dL. In some embodiments, the reducing is to a normal level. In some embodiments, the normal level ranges from 2 mg/dL to 30 mg/dL (0.1 to 1.7 mmol/L).

An “abnormally high concentration” of NEFA is in a subject's blood plasma or blood serum in a non-fasting state is 0.9 mM or greater. An “abnormally high concentration” of NEFA in a subject's blood plasma or blood serum in a fasting state is greater than 1.8 mM at a fasting state. An “abnormally high concentration” of NEFA in a subject's blood plasma or blood serum at 15-hour fasting is greater than 1.1 nM. An “abnormally high concentration” of NEFA in a subject's blood plasma or blood serum at 20-hour fasting is greaterthan 1.3 mM. An “abnormally high concentration” of NEFA in a subject's blood plasma or blood serum at 15-hour fasting is greater than 1.1 nM. An “abnormally high concentration” of NEFA in a subject's blood plasma or blood serum at 24-hour fasting is greater than 1.8 mM. In some embodiments, the present invention provides methods for reducing NEFA concentration in a subject's blood plasma or blood serum, wherein the NEFA concentration is greater than 0.9 mM, in some embodiments greater than 1.1 mM, in some embodiments greater than 1.5 mM and in some embodiments greater than 1.8 mM. In some embodiments, the reducing is to a normal level. In some embodiments, the normal level is 1.8 mM or less, in some embodiments 1.5 mM or less, in some embodiments 1.1 mM or less and in some embodiments 0.9 mM or less. See Horowitz, G. L. et al. Medscape emedicine, updated Jul. 25, 2019.

In some embodiments, the present invention provides methods for treating or preventing dyslipoproteinemia, comprising administering to a subject in need thereof an effective amount of a compound of the invention or a composition of the invention. In some embodiments, the dyslipoproteinemia is characterized by an abnormally high concentration of LDL, apolipoprotein (a) or VLDL in a subject's blood plasma or blood serum, or an abnormally low concentration of high density lipoprotein (HDL) or lipoprotein lipase in a subject's blood plasma or blood serum. In some embodiments, the abnormally low concentration of the lipoprotein lipase is associated with: a lipoprotein lipase mutation, hypoalphalipoproteinemia, a lipoprotein abnormality associated with diabetes, a lipoprotein abnormality associated with obesity, a lipoprotein abnormality associated with Alzheimer's disease, or familial combined hyperlipidemia. The term “dyslipoproteinemia” refers to a disorder that leads to or is manifested by an aberrant concentration of circulating lipoproteins in a subject's blood plasma or blood serum. To the extent that the concentrations of lipoproteins in the blood plasma or blood serum are too high, the compounds of the invention or the compositions of the invention can be administered to the subject to restore to normal concentrations of lipoproteins. Conversely, to the extent that the concentrations of lipoproteins in the blood plasma or blood serum are too low, the compounds of the invention or the compositions of the invention can be administered to the subject to restore to normal concentrations. Normal concentrations of lipoproteins are reported in medical treatises known to those of skill in the art.

In some embodiments, the present invention provides methods for treating or preventing a renal disease, comprising administering to a subject in need thereof an effective amount of a compound of the invention or a composition of the invention. In some embodiments, the renal disease is a glomerular disease, a tubular disease, a tubulointerstitial disease, acute or rapidly progressive renal failure, chronic renal failure, nephrolithiasis, or a tumor. In some embodiments, the renal disease is hypertension, nephrosclerosis, microangiopathic hemolytic anemia, atheroembolic renal disease, diffuse cortical necrosis, or a renal infarct.

In some embodiments, the glomerular disease is an acute glomerulonephritis, a chronic glomerulonephritis, a rapidly progressive glomerulonephritis, a nephrotic syndrome, a focal proliferative glomerulonephritis, a glomerular lesion associated with systemic disease, Goodpasture syndrome, multiple myeloma, diabetes, neoplasia, sickle cell disease or a chronic inflammatory disease. In some embodiments, the glomerular lesion associated with systemic disease is systemic lupus erythematosus.

In some embodiments, the tubular disease is an acute tubular necrosis, an acute renal failure, a polycystic renal disease, medullary sponge kidney, a medullary cystic disease, nephrogenic diabetes, or a renal tubular acidosis.

In some embodiments, the tubulointerstitial disease is pyelonephritis, a drug- or toxin-induced tubulointerstitial nephritis, a hypercalcemic nephropathy, or a hypokalemic nephropathy.

In some embodiments, the tumor is renal cell carcinoma or nephroblastoma.

In some embodiments, the renal disease is hypertension. In some embodiments, the hypertension is an essential hypertension, hyperpiesa, hyperpiersis, a malignant hypertension, a secondary hypertension, or a white-coat hypertension.

In some embodiments, the renal disease is a kidney disease.

In some embodiments, the present invention provides methods for treating or preventing a disorder of glucose metabolism, comprising administering to a subject in need thereof an effective amount of a compound of the invention or a composition of the invention. The term “disorder of glucose metabolism” refers to a disorder that leads to or is manifested by aberrant glucose storage and/or utilization. To the extent that indicia of glucose metabolism (i.e. insulin, glucose, or glycated hemoglobin in a subject's blood plasma or blood serum) are too high, the compounds of the invention or the compositions of the invention can be administered to a subject to restore to normal levels. Normal indicia of glucose metabolism are reported in medical treatises known to those of skill in the art. See U.S. Pat. No. 7,709,682 B2.

In some embodiments, the present invention provides methods for reducing an abnormally high concentration of glucose in a subject's blood plasma or blood serum, comprising administering to a subject in need thereof an effective amount of a compound of the invention or a composition of the invention. An “abnormally high concentration” of glucose in a subject's blood plasma or blood serum at a fasted state (10-16 hours without eating) is greater than 5.6 mmol/L (100 mg/dL). In some embodiments, the reducing is to a normal concentration. In some embodiments, the normal concentration of glucose is less than 5.6 mmol/L at fasted state. In some embodiments, a fasted glucose blood plasma or blood serum concentration in the range of 5.6 mmol/L to 6 mmol/L (100-109 mg/dL) may indicate prediabetes. In some embodiments, a fasted glucose blood plasma or blood serum concentration in the range of 6.1 mmol/L to 6.9 mmol/L (110-125 mg/dL) can indicate diabetes. In some embodiments, a fasted glucose blood plasma or blood serum concentration of 7 mmol/L (126 mg/dL) and above indicates diabetes.

In some embodiments, the abnormally high concentration of glucose in a subject's blood plasma or blood serum is measured in a glucose tolerance test (GTT).

An “abnormally high concentration” of glucose in a subject's blood plasma or blood serum in a one-hour GTT is greater than 10 mmol/L (180 mg/dL). In some embodiments, the reducing is to a normal concentration. In some embodiments, the normal concentration in a one-hour GTT is less than 10 mmol/L (180 mg/dL).

An “abnormally high concentration” of glucose in a subject's blood plasma or blood serum in a two-hour GTT with 75 g intake is greater than 7.8 mmol/L (140 mg/dL), which indicates hyperglycemia. In some embodiments, the reducing is to a normal concentration. In some embodiments, the normal concentration in two-hour GTT with 75 g intake is less than 7.8 mmol/L (140 mg/dL). In some embodiments, a glucose concentration in a subject's blood plasma or blood serum between 7.8 mmol/L (140 mg/dL) and 11.1 mmol/L (200 mg/dL) in two-hour GTT with 75 g intake indicates impaired glucose tolerance. In some embodiments, a glucose concentration above 11.1 mmol/L in two-hour GTT with 75 g intake indicates diabetes.

In some embodiments, the present invention provides methods for increasing abnormally low glucose metabolism in a subject, wherein the subject's glucose concentration in the subject's blood plasma or blood serum is greater than 7.8 mmol/L (140 mg/dL) in a two-hour GTT. In some embodiments, the present invention provides methods treating or preventing a disorder of glucose metabolism in a subject, wherein the subject's glucose concentration in the subject's blood plasma or blood serum is in the range of 7.8 mmol/L (140 mg/dL) to 11.1 mmol/L (200 mg/dL) in a two-hour GTT. In some embodiments, the present invention provides methods for treating or preventing a disorder of glucose metabolism in a subject, wherein the subject's glucose concentration in the subject's blood plasma or blood serum is greater than 11.1 mmol/L (200 mg/dL) in a two-hour GTT.

In some embodiments, the present invention provides methods for reducing an abnormally high level of HbA_{1c} in a subject's blood plasma or blood serum, comprising administering to a subject in need thereof an effective amount of a compound of the invention or a composition of the invention. An “abnormally high level” of hemoglobin A1c (HbA_{1c}) in a subject's blood plasma or blood serum is 6.5% or greater (expressed in % NGSP units). In some embodiments, the reducing is to a normal level. In some embodiments, the normal levels of HbA_{1c} is in the range of about 4% to about 5.9%. In some embodiments, the present invention provides methods for reducing HbA_{1c} level in a subject's blood plasma or blood serum, wherein the HbA_{1c} level is greater than 7%, greater than 8%, or greater than 9%. See Horowitz, G. L. et al. Medscape emedicine, updated Jul. 25, 2019.

In some embodiments, the present invention provides methods for increasing abnormally low glucose metabolism in a subject, wherein the subject's HbA_{1c} level is 6.5% or greater and the subject's fasting glucose concentration is 126 mg/dL or greater (≥7.0 mmol/L), in the subject's blood plasma or blood serum. See Selvin, E. et al. Ann Intern Med. Published online Jun. 18, 2018.

In some embodiments, the present invention provides methods for treating or preventing a disorder of glucose metabolism in a subject, wherein the subject has HbA_{1c} greater than or equal to 6.5%. In some embodiments, the present invention provides methods for treating or preventing a disorder of glucose metabolism in a subject, wherein the subject has HbA_{1c} greater than or equal to 6.5% and fasting glucose concentration greater than or equal to 126 mg/dL (7.0 mmol/L) in the subject's blood plasma or blood serum.

In some embodiments, the present invention provides methods for reducing an abnormally high concentration of glucose in a subject's blood plasma or blood serum, comprising administering to a subject in need thereof an effective amount of a compound of the invention or a composition of the invention, wherein the subject is pregnant. An “abnormally high concentration” of glucose in a pregnant subject's blood plasma or blood serum at fasted state is greater than 5.3 mmol/L (95 mg/dL).

In some embodiments, the present invention provides methods for reducing an abnormally high concentration of glucose in a subject's blood plasma or blood serum, comprising administering to a subject in need thereof (i) a glucose solution as part of a two-step gestational diabetes test and (ii) an effective amount of a compound of the invention or a composition of the invention. An “abnormally high concentration” of glucose in a subject's blood plasma or blood serum at 1 hour after drinking the glucose solution in a two-step gestational diabetes test is greater than 10 mmol/L (180 mg/dL). In the two-step procedure, the first step is a 50 g glucose dose. If it results in a blood glucose level of more than 7.8 mmol/L (140 mg/dL), it is followed by a 100 g glucose dose. An “abnormally high concentration: of glucose in a subject's blood plasma or blood serum at 2 hour after drinking the glucose solution in a two-step gestational diabetes test is greater than 8.6 mmol/L (155 mg/dL). An “abnormally high concentration: of glucose in a subject's blood plasma or blood serum at 3 hour after drinking the glucose solution in a two-step gestational diabetes test is greater than 7.8 mmol/L (140 mg/dL).

In some embodiments, the present invention provides methods for treating or preventing a disorder of glucose metabolism in a subject, wherein the subject has impaired glucose tolerance. In some embodiments, the present invention provides methods for treating or preventing a disorder of glucose metabolism in a subject, wherein the subject has diabetes. In some embodiments, the present invention provides methods for treating or preventing a disorder of glucose metabolism in a subject, wherein the subject has confirmed undiagnosed diabetes. In some embodiments, the present invention provides methods for treating or preventing a disorder of glucose metabolism in a subject, wherein the subject has gestational diabetes.

In some embodiments, the present invention provides methods for reducing abnormally high concentration of insulin in a subject's blood plasma or blood serum, comprising administering to a subject in need thereof an effective amount of a compound of the invention or a composition of the invention. An “abnormally high concentration” of insulin in a subject's blood plasma or blood serum at a fasted state is greater than 25 mlU/L (>174 μmol/L). In some embodiments, the reducing is to a normal concentration. In some embodiments, the normal concentration of insulin in a subject's blood plasma or blood serum at a fasted state is less than 25 mlU/L (<174 μmol/L). See Buppajarntham, S. et al. Medscape emedicine, updated Jan. 2, 2019.

In some embodiments, the present invention provides methods for reducing abnormally high concentration of insulin in a subject's blood plasma or blood serum, comprising administering to a subject in need thereof an effective amount of a compound of the invention or a composition of the invention.

In some embodiments, the methods further comprise administering glucose to the subject. In some embodiments, the methods do not comprise administering glucose to the subject. In some embodiments, the subject is in a fasted state.

In some embodiments, the subject has an abnormally concentration of insulin in the subject's blood plasma or blood glucose at 30 minutes after glucose administration. An “abnormally high concentration” of insulin in a subject's blood plasma or blood serum at 30 minutes after glucose administration is greater than 230 mlU/L (>1597 μmol/L). In some embodiments, the reducing is to a normal concentration. In some embodiments, the normal concentration of insulin in a subject's blood plasma or blood serum at 30 minutes after glucose administration is in the range of about 30 mlU/L to about 230 mlU/L (208-1597 μmol/L). See Buppajarntham, 2019.

In some embodiments, the subject has an abnormally concentration of insulin in the subject's blood plasma or blood serum at 1 hour after glucose administration. An “abnormally high concentration” of insulin in a subject's blood plasma or blood serum at 1 hour after glucose administration is greater than 276 mlU/L (>1917 μmol/L). In some embodiments, the present invention provides methods for reducing abnormally high concentration of insulin in a subject's blood plasma or blood serum, comprising administering to a subject in need thereof an effective amount of a compound of the invention or a composition of the invention. In some embodiments, the reducing is to a normal concentration. In some embodiments, the normal concentration of insulin in a subject's blood plasma or blood serum at 1 hour after glucose administration is in the range of about 18 mlU/L to about 276 mlU/L (125-1917 μmol/L). See Buppajarntham, 2019.

In some embodiments, the subject has an abnormally concentration of insulin in the subject's blood plasma or blood glucose at 2 hours after glucose administration. An “abnormally high concentration” of insulin in a subject's blood plasma or blood serum at 2 hour after glucose administration is greater than 166 mlU/L (>1153 μmol/L). In some embodiments, the present invention provides methods for reducing an abnormally high concentration of insulin in a subject's blood plasma or blood serum, comprising administering to a subject in need thereof an effective amount of a compound of the invention or a composition of the invention. In some embodiments, the normal concentration of insulin in a subject's blood plasma or blood serum at 2 hours after glucose administration is in the range of about 16 mlU/L to about 166 mlU/L (111-1153 μmol/L). See Buppajarntham, 2019.

In some embodiments, the subject has an abnormally concentration of insulin in the subject's blood plasma or blood glucose at 3 hours after glucose administration. An “abnormally high concentration” of insulin in a subject's blood plasma or blood serum at 3 hours after glucose administration is greater than 25 mlU/L (>174 μmol/L). In some embodiments, the present invention provides methods for reducing an abnormally high concentration of insulin in a subject's blood plasma or blood serum, comprising administering to a subject in need thereof an effective amount of a compound of the invention or a composition of the invention. In some embodiments, the normal concentration of insulin in a subject's blood plasma or blood serum at 3 hours or later after glucose administration is less than 25 mlU/L (<174 μmol/L). See Buppajarntham, 2019.

In some embodiments, the present invention provides methods for treating or preventing a disorder of glucose metabolism in a subject, wherein the subject has insulin concentration in the subject's blood plasma or blood serum above 25 mlU/L at a fasted state or after 3 hours after glucose administration. See Buppajarntham, 2019.

In some embodiments, the disorder of glucose metabolism is an impaired glucose tolerance; an insulin resistance; an insulin resistance-related breast, colon or prostate cancer; diabetes; pancreatitis; hypertension; polycystic ovarian disease; or an abnormally high concentration of blood insulin or glucose in the subject's blood plasma or blood serum. In some embodiments, the diabetes is non-insulin dependent diabetes mellitus (NIDDM), insulin dependent diabetes mellitus (IDDM), gestational diabetes mellitus (GDM), or maturity onset diabetes of the young (MODY).

In some embodiments, the present invention provides methods for treating or preventing a metabolic syndrome (syndrome X), comprising administering to a subject in need thereof an effective amount of a compound of the invention or a composition of the invention. In some embodiments, the present invention provides methods for treating or preventing a symptom of a metabolic syndrome (syndrome X), comprising administering to a subject in need thereof an effective amount of a compound of the invention or a composition of the invention. In some embodiments, the symptom is impaired glucose tolerance, hyper tension, dyslipidemia, or dyslipoproteinemia.

In some embodiments, the present invention provides methods for treating or preventing a vascular disease or cardiovascular disease, comprising administering to a subject in need thereof an effective amount of a compound of the invention or a composition of the invention. The term “cardiovascular disease” refers to a disease of the heart or circulatory system. In some embodiments, the vascular disease or the cardiovascular disease is a peripheral vascular disease, a coronary heart disease, stroke, restenosis, arteriosclerosis, ischemia, an endothelium dysfunction, an ischemia-reperfusion injury, a myocardial infarction, or a cerebral infarction.

In some embodiments, the present invention provides methods for treating or preventing a PPAR-associated disorder, comprising administering to a subject in need thereof an effective amount of a compound of the invention or a composition of the invention. In some embodiments, the PPAR-associated disorder is rheumatoid arthritis, multiple sclerosis, psoriasis, an inflammatory bowel disease, breast cancer, colon cancer, or prostate cancer. In some embodiments, the PPAR-associated disorder is a vascular disease, a muscular disease, a demyelinating disease, a muscle structure disorder, a neuronal activation disorder, a muscle fatigue disorder, a muscle mass disorder, a mitochondrial disease, a mitochondrial dysfunction, a beta oxidation disease, or a metabolic disease. In some embodiments, the PPAR-associated disorder is an abnormally low concentration of HDL, an abnormally low concentration of apolipoprotein A-I (apo A-I), an abnormally high concentration of VLDL-C, an abnormally high concentration of low density lipoprotein cholesterol (LDL-C), an abnormally high concentration of triglyceride, an abnormally high concentration of apolipoprotein B (apo B), an abnormally high concentration of apolipoprotein C-III (apo C-III) or an abnormally reduced ratio of post-heparin hepatic lipase to lipoprotein lipase activity in the subject's blood plasma or blood serum. In some embodiments, the PPAR-associated disorder is an abnormally high concentration of HDL or an abnormally low concentration of apo A-I in the subject's lymph or cerebral fluid.

In some embodiments, the PPAR-associated disorder is abnormally low concentration of lipoprotein lipase activity in the post-heparin plasma in a subject (subject's blood plasma after intravenous injection of heparin). In some embodiments, the present invention provides methods for elevating an abnormally low concentration of lipoprotein lipase activity in the post-heparin plasma, comprising administering to a subject in need thereof an effective amount of a compound of the invention or a composition of the invention. An “abnormally low concentration” of lipoprotein lipase activity in the post-heparin plasma is less than 30 U/L. In some embodiments, the elevating is to a normal concentration. In some embodiments, the normal concentration is in the range from about 30 U/L to about 153 U/L (See Nakajima et al. Clin Chim Acta. 2018 December; 487:54-59).

In some embodiments, the present invention provides methods for treating or preventing a PPAR-associated disorder in a subject, wherein the subject has lipoprotein lipase activity in the post-heparin plasma of less than 30 U/L.

In some embodiments, the subject is a male subject. An “abnormally high concentration” of HDL in a male subject is greater than 75 mg/dL.

In some embodiments, the subject is a female subject. An “abnormally high concentration” of HDL for a female subject is greater than 90 mg/dL.

In some embodiments, the present invention provides methods for reducing an abnormally high concentration of HDL in a subject's blood plasma or blood serum, comprising administering to a subject in need thereof an effective amount of a compound of the invention or a composition of the invention. In some embodiments, the reducing is to a normal concentration. In some embodiments, the normal concentration for a male subject is less than 75 mg/dL. In some embodiments, the normal concentration for a female subject is less than 90 mg/dL. In some embodiments, the present invention provides methods for treating or preventing a PPAR-associated disorder in a male subject, wherein the subject has HDL concentration in the subject's blood plasma or blood serum greater than 75 mg/dL. In some embodiments, the present invention provides methods for treating or preventing a PPAR-associated disorder in a female subject, wherein the subject has HDL concentration in the subject's blood plasma or blood serum greater than 90 mg/dL. See Hassan, M. et. al. Glob Cardiol Sci Pract. 2016 Dec. 30; 2016(4): e201634.

In some embodiments, the muscular disease is a muscular dystrophy disease. In some embodiments, the muscular dystrophy disease is Duchenne muscular dystrophy, Becker muscular dystrophy, a limb-girdle muscular dystrophy, congenital muscular dystrophy, facioscapulohumeral muscular dystrophy, myotonic muscular dystrophy, oculopharyngeal muscular dystrophy, distal muscular dystrophy, or Emery-Dreifuss muscular dystrophy.

In some embodiments, the demyelinating disease is multiple sclerosis, Charcot-Marie-Tooth disease, Pelizaeus-Merzbacher disease, encephalomyelitis, neuromyelitis optica, adrenoleukodystrophy, or Guillian-Barre syndrome.

In some embodiments, the muscle structure disorder is Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorder, myotonic chondrodystrophy, myotonic dystrophy, myotubular myopathy, nemaline body disease, oculopharyngeal MD, or stress urinary incontinence.

In some embodiments, the neuronal activation disorder is amyotrophic lateral sclerosis, Charcot-Marie-Tooth disease, Guillain-Barre syndrome, Lambert-Eaton syndrome, multiple sclerosis, myasthenia gravis, a nerve lesion, peripheral neuropathy, spinal muscular atrophy, tardy ulnar nerve palsy, or toxic myoneural disorder.

In some embodiments, the muscle fatigue disorder is chronic fatigue syndrome, diabetes (type I or II), a glycogen storage disease, fibromyalgia, Friedreich's ataxia, intermittent claudication, lipid storage myopathy, MELAS (mitochondrial encephalopathy, lactic acidosis, and stroke-like episodes) syndrome, mucopolysaccharidosis, Pompe disease, or thyrotoxic myopathy.

In some embodiments, the muscle mass disorder is cachexia, cartilage degeneration, cerebral palsy, compartment syndrome, critical illness myopathy, inclusion body myositis, muscular atrophy (disuse), sarcopenia, steroid myopathy, or systemic lupus erythematosus.

In some embodiments, the mitochondrial disease is Alpers's disease, chronic progressive external ophthalmoplegia (CPEO), Kearns-Sayra syndrome (KSS), Leber hereditary optic neuropathy (LHON), MELAS, myoclonic epilepsy and ragged-red fiber disease (MERRF), neurogenic muscle weakness (NARP), ataxia, retinitis pigmentosa, Pearson syndrome, a mitochondrial malfunction, or a mitochondrial loss of functionality (for example, due to a drug affecting mitochondrial functions).

In some embodiments, the mitochondrial dysfunction is a drug induced mitochondrial dysfunction.

In some embodiments, the beta oxidation disease is systemic camitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, or riboflavin-responsive disorders of β-oxidation (RR-MADD).

In some embodiments, the metabolic disease is hyperlipidemia, dyslipidemia, hyperchlolesterolemia, hypertriglyceridemia, HDL hypocholesterolemia, LDL hypercholesterolemia, HLD non-cholesterolemia, VLDL hyperproteinemia, dyslipoproteinemia, apolipoprotein A-I hypoproteinemia, atherosclerosis, a disease of arterial sclerosis, a disease of cardiovascular system, cerebrovascular disease, peripheral circulatory disease, metabolic syndrome, syndrome X, obesity, diabetes, type I diabetes, type II diabetes, hyperglycemia, insulin resistance, impaired glucose tolerance, hyperinsulinism, a diabetic complication, cardiac insufficiency, cardiac infarction, cardiomyopathy, hypertension, non-alcoholic fatty liver disease (NAFLD), non-alcoholic steatohepatitis (NASH), a thrombus, Alzheimer disease, a neurodegenerative disease, a demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, or pancreatitis.

In some embodiments, the present invention provides methods for treating or preventing septicemia, comprising administering to a subject in need thereof an effective amount of a compound of the invention or a composition of the invention. In some embodiments, septicemia is septic shock.

In some embodiments, the present invention provides methods for treating or preventing a thrombotic disorder, comprising administering to a subject in need thereof an effective amount of a compound of the invention or a composition of the invention. In some embodiments, the thrombotic disorder is high concentration of fibrinogen in the subject's blood plasma or blood serum, or promotion of fibrinolysis.

In some embodiments, the present invention provides methods for treating or preventing obesity, comprising administering to a subject in need thereof an effective amount of a compound of the invention or a composition of the invention. In some embodiments, the obesity is abdominal obesity. In some embodiments, the methods for treating or preventing obesity further comprise promoting weight reduction in the subject.

In some embodiments, the present invention provides methods for treating or preventing diabetic nephropathy, comprising administering to a subject in need thereof an effective amount of a compound of the invention or a composition of the invention. In some embodiments, the methods for treating or preventing diabetic nephropathy further comprise treating or preventing a kidney disease that develops as a result of diabetes mellitus. In some embodiments, the present invention provides methods for treating or preventing diabetes mellitus, comprising administering to a subject in need thereof an effective amount of a compound of the invention or a composition of the invention.

In some embodiments, the present invention provides methods for treating or preventing diabetic retinopathy, comprising administering to a subject in need thereof an effective amount of a compound of the invention or a composition of the invention. In some embodiments, the methods for treating or preventing diabetic retinopathy result in treating or preventing a complication of diabetes that can lead to or cause blindness.

In some embodiments, the present invention provides methods for treating or preventing a cerebrovascular disease, comprising administering to a subject in need thereof an effective amount of a compound of the invention or a composition of the invention. In some embodiments, the cerebrovascular disease is cerebral ischemia.

In some embodiments, the present invention provides methods for treating or preventing a disorder related to neovascularization, comprising administering to a subject in need thereof an effective amount of a compound of the invention or a composition of the invention. In some embodiments, the disorder related to neovascularization is retinopathy or diabetes.

In some embodiments, the present invention provides methods for treating or preventing hypertension, comprising administering to a subject in need thereof an effective amount of a compound of the invention or a composition of the invention. In some embodiments, the methods for treating or preventing hypertension result in treating or preventing blood flow that occurs through the subject's vessels at a greater than normal force.

In some embodiments, the present invention provides methods for treating or preventing cancer, comprising administering to a subject in need thereof an effective amount of a compound of the invention or a composition of the invention. In some embodiments, the cancer is a human sarcoma or human carcinoma. In some embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, bone cancer, breast cancer, ovarian cancer, prostate cancer, esophageal cancer, oral cancer, nasal cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, leukemia, acute lymphoblastic B-cell leukemia, acute lymphoblastic T-cell leukemia, acute promyelocytic leukemia, acute monoblastic leukemia, acute erythroleukemic leukemia, acute megakaryoblastic leukemia, acute myelomonocytic leukemia, acute nonlymphocytic leukemia, acute undifferentiated leukemia, chronic myelocytic leukemia, hairy cell leukemia, lymphoblastic leukemia, myelogenous leukemia, lymphocyticleukemia, myelocytic leukemia, polycythemia vera, multiple myeloma, lymphoma, Hodgkin's disease, non-Hodgkin's lymphoma, Waldenstrom's macroglobulinemia, or a heavy chain disease.

In some embodiments, the leukemia is acute or chronic lymphoblastic leukemia, myelogenous leukemia, lymphocyticleukemia, lymphocytic leukemia, or myelocytic leukemia. In some embodiments, the myelocytic leukemia is acute and is myeloblastic, promyclocytic, myelomonocytic, monocytic or erythroleukemia

In some embodiments, the lymphoma is Hodgkin's lymphoma or non-Hodgkin's lymphoma.

In some embodiments, the present invention provides methods for treating or preventing an inflammatory disease, comprising administering to a subject in need thereof an effective amount of a compound of the invention or a composition of the invention. In some embodiments, the inflammatory disease is multiple sclerosis, a chronic inflammatory disorder of a joint, arthritis, a respiratory distress syndrome, an inflammatory bowel disease, an inflammatory lung disorder, an inflammatory disorder, an inflammatory disorder of the gum, tuberculosis, leprosy, an inflammatory disease of the kidney, an inflammatory disorder of the skin, an inflammatory disease of the central nervous system, a systemic lupus erythematosus (SLE) or an inflammatory disease of the heart.

In some embodiments, the arthritis is rheumatoid arthritis or osteoarthritis.

In some embodiments, the inflammatory bowel disease is ileitis, ulcerative colitis or Crohn's disease.

In some embodiments, the inflammatory lung disorder is asthma or chronic obstructive airway disease.

In some embodiments, the inflammatory disorder of the eye is corneal dystrophy, trachoma, onchocerciasis, uveitis, sympathic ophthalmitis or endophthalmitis.

In some embodiments, the inflammatory disorder of the gum is periodontitis or gingivitis.

In some embodiments, the inflammatory disease of the kidney is glomerulonephritis or nephrosis.

In some embodiments, the inflammatory disorder of the skin is acne, sclerodermatitis, psoriasis, eczema, photoaging or wrinkles.

In some embodiments, the inflammatory disease of the central nervous system is AIDS-related neurodegeneration, stroke, neurotrauma, Alzheimer's disease, encephalomyelitis, or viral or autoimmune encephalitis.

In some embodiments, the inflammatory disease of the heart is cardiomyopathy.

In some embodiments, the present invention provides methods for treating or preventing a neurodegenerative disease, comprising administering to a subject in need thereof an effective amount of a compound of the invention or a composition of the invention. In some embodiments, the neurodegenerative disease is Alzheimer's disease or Huntington's disease.

In some embodiments, the present invention provides methods for treating or preventing an autoimmune disease, comprising administering to a subject in need thereof an effective amount of a compound of the invention or a composition of the invention. In some embodiments, the autoimmune disease is immune-complex vasculitis, systemic lupus or erythematodes.

In some embodiments, the present invention provides methods for treating or preventing a neoplastic disease, comprising administering to a subject in need thereof an effective amount of a compound of the invention or a composition of the invention. In some embodiments, the neoplastic disease is carcinogenesis.

In some embodiments, the present invention provides methods for treating or preventing cholestasis, comprising administering to a subject in need thereof an effective amount of the compound of the invention or the composition of the invention.

In some embodiments, the cholestasis is intrahepatic cholestatic disease or extrahepatic cholestatic disease. In some embodiments, the intrahepatic cholestatic disease is primary biliary cholangitis (PBC), primary sclerosing cholangitis (PSC), progressive familial intrahepatic cholestasis (PFIC), or Alagille syndrome (AS). In some embodiments, the methods for treating or preventing intrahepatic cholestatic disease result in preventing or reducing the risk of developing an intrahepatic cholestatic disease, e.g., causing the clinical symptoms of an intra hepatic cholestatic disease to not develop in a subject who may be predisposed to an intrahepatic cholestatic disease by who does not yet experience or display symptoms of the intrahepatic cholestatic disease (i.e., prophylaxis). In some embodiments, the methods for treating or preventing intrahepatic cholestatic disease comprise inhibiting an intrahepatic cholestatic disease, e.g., arresting or reducing the development of the intrahepatic cholestatic disease or reducing the number, frequency, duration or severity of one or more of its clinical symptoms.

In some embodiments, the present invention provides methods for treating or preventing an ocular disease, comprising administering to a subject in need thereof an effective amount of a compound of the invention or a composition of the invention. In some embodiments, the ocular disease is dry eye, meibomian gland dysfunction, a keratoconjunctiva epithelial disorder, a corneal epithelial disorder, or a corneal ulcer. In some embodiments, the ocular disease is dry eye syndrome, corneal ulcer, superficial punctuate keratitis, corneal epithelial erosion, an ocular allergic disease associated with corneal lesion such as vernal conjunctivitis, or atopic keratoconjunctivitis. In some embodiments, the ocular disease is hyperevaporative dry eye. In some embodiments, the ocular disease is injury of corneal epithelial cells. In some embodiments, the injury of corneal epithelial cells is associated with endogenous diseases such as Sjogren's syndrome, Stevens-Johnson syndrome, keratoconjunctivitis sicca (dry eye) or the like. In some embodiments, the injury of corneal epithelial cells is associated with exogenous diseases such as post-operation, drug use, trauma, corneal ulcer, meibomianitis, exogenous diseases during wearing contact lenses or the like. In some embodiments, the injury of corneal epithelial cells is associated with ocular allergic diseases accompanying corneal lesion such as vernal conjunctivitis, atopic keratoconjunctivitis or the like. In some embodiments, the ocular disease is superficial punctuate keratitis and corneal epithelial erosion.

In some embodiments, the methods for treating or preventing an ocular disease result in promoting proliferation of meibomian gland epithelial cells and corneal epithelial cells.

In some embodiments, the present invention provides methods for treating or preventing a lysosomal storage disorder, comprising administering to a subject in need thereof an effective amount of a compound of the invention or a composition of the invention. In some embodiments, the lysosomal storage disorder is neuronal ceroid lipofuscinosis, Alzheimer's disease, Huntington's disease, amyotrophic lateral sclerosis (ALS), Parkinson's disease, multiple system atrophy (MSA), progressive supranuclear palsy (PSP), corticobasal degeneration (CBD), dementia with Lewy bodies (DLB), a disorder of the autophagy pathway, Tay-Sach's disease, Fabry disease, Niemann-Pick disease, Gaucher disease, Hunter syndrome, alpha-mannosidosis, aspartylglucosaminuria, cholesteryl ester storage disease, chronic hexosaminidase A deficiency, cystinosis, Danon disease, Farber disease, fucosidosis, galactosialidosis, or Batten disease.

In some embodiments, the present invention provides methods for treating or preventing a kidney disease, comprising administering to a subject in need thereof an effective amount of a compound of the invention or a composition of the invention. In some embodiments, the kidney disease is renal ischemia reperfusion injury. In some embodiments, the kidney disease is acute kidney injury. In some embodiments, the methods for treating or preventing a kidney disease result in lowering the subject's risk for acute kidney injury following coronary artery bypass graft, transplantation, and/or valve surgery.

In some embodiments, the present invention provides methods for treating or preventing impotence, comprising administering to a subject in need thereof an effective amount of a compound of the invention or a composition of the invention. In some embodiments, the impotence results from damages to a nerve, artery, smooth muscles, or fibrous tissue; diabetes; kidney disease; alcoholism; multiple sclerosis; atherosclerosis; vascular disease; or neurologic disease. In some embodiments, the methods for treating or preventing impotence results in treating or preventing erectile dysfunction.

The present invention provides methods for treating or preventing hyperlipemia, hyperlipidemia, hyperlipoproteinemia, hypercholesterolemia, hypertriglyceridemia, or dyslipidemia, comprising administering to a subject in need thereof an effective amount of a compound of the invention or a composition of the invention. In some embodiments, the hypercholesterolemia is homozygous familial hypercholesterolemia.

The present invention provides methods for treating a subject having or preventing a subject from having an abnormally high concentration in a subject's blood plasma or blood serum of high low-density lipoprotein (LDL), apolipoprotein B (apo B), lipoprotein(a) (Lp(a)), apolipoprotein (a), or very low-density lipoprotein (VLDL), comprising administering to a subject in need thereof an effective amount of a compound of the invention or a composition of the invention.

An “abnormally high concentration” of lipoprotein-cholesterol can depend on the number of risk factors and whether the treatment is for a primary or a secondary prevention. As used herein a “primary prevention” refers to a treatment aimed to avoid a subject developing or getting a disease or a condition. As used herein a “secondary prevention” refers to a treatment aimed to detect a disease or a condition early and prevent the disease or condition from getting worse or advancing. Recommended lipoprotein-cholesterol concentrations can be found in guidelines published by the National Lipid Association or by the National Institute of Health National Heart, Lung, and Blood Institute such as the Third Report of the Expert Panel on Detection, Evaluation, and Treatment of High Blood Cholesterol in Adults (ATP III Final Report), 2002. The ATP III Final Report is hereby incorporated by reference in its entirety for all purposes.

In some embodiments, the present invention provides methods for reducing an abnormally high concentration of apo B in a subject's blood plasma or blood serum, comprising administering to a subject in need thereof an effective amount of a compound of the invention or a composition of the invention. In some embodiments, an “abnormally high concentration” of apo B in a subject's blood plasma or blood serum is greater than 130 mg/dL. In some embodiments, the reducing is to a normal concentration. In some embodiments, the normal concentration of apo B in a subject's blood plasma or blood serum is less than 130 mg/dL. In some embodiments, the subject is a male subject. In some embodiments, the subject is a female subject. In some embodiments, the present invention provides methods for treating a subject with apo B blood plasma or blood serum concentration of greaterthan 130 mg/dL. In some embodiments, the present invention provides methods for treating a subject with apo B blood plasma or blood serum concentration of greater than 130 mg/dL, when the subject is at low risk of coronary heart disease (CHD) having 0-1 CHD risk factors.

In some embodiments, the present invention provides methods for treating a subject with apo B blood plasma or blood serum concentration of greater than 110 mg/dL, when the subject is at moderate risk of CHD having 2 or more CHD risk factors. In some embodiments, an “abnormally high concentration” of apo B in a subject's blood plasma or blood serum is greater than 110 mg/dL. In some embodiments, the subject has 2 or more CHD risk factors. In some embodiments, the subject has a CHD or a CHD risk equivalent. In some embodiments, the present invention provides methods for treating a subject with apo B blood plasma or blood serum concentration of greater than 90 mg/dL, when the subject has a CHD or a CHD risk equivalent.

In some embodiments, the present invention provides methods for reducing an abnormally high concentration of Lp(a) in a subject's blood plasma or blood serum, comprising administering to a subject in need thereof an effective amount of a compound of the invention or a composition of the invention. An “abnormally high concentration” of Lp(a) in a subject's blood plasma or blood serum is greater than 10 mg/dL. In some embodiments, the abnormally high concentration of Lp(a) is associated with an increase in cardiovascular risk. In some embodiments, the reducing is to a normal concentration. In some embodiments, the normal concentration of Lp(a) in a subject's blood plasma or blood serum is less than 10 mg/dL. In some embodiments, the normal concentration of Lp(a) in a subject's blood plasma or blood serum is less than 50 mg/dL (See, Banach, M. J Am Heart Assoc. 2016 April; 5(4): e003597). In some embodiments, the present invention provides methods for treating a subject with Lp(a) blood plasma concentration of greater than 50 mg/dL, comprising administering to a subject in need thereof an effective amount of a compound of the invention or a composition of the invention.

The present invention provides methods for treating a subject having or preventing a subject from having an abnormally high apo B/apo A-I ratio in a subject's blood plasma or blood serum, comprising administering to a subject in need thereof an effective amount of a compound of the invention or a composition of the invention. In some embodiments, the present invention provides methods for reducing an abnormally high apo B/apo A-I ratio in a subject's blood plasma or blood serum, comprising administering to a subject in need thereof an effective amount of a compound of the invention or a composition of the invention. An “abnormally high” apo B/apo A-I ratio in a subject's blood plasma or blood serum is greater than 0.9. In some embodiments, the reducing is to a normal level. In some embodiments, a normal apo B/apo A-I ratio in a subject's blood plasma or blood serum is less than 0.9. In some embodiments, the normal apo B/apo A-I ratio in a subject's blood plasma or blood serum is less than 0.7. In some embodiments, the subject has an apo B/apo A-I ratio in a subject's blood plasma or blood serum of greater than 0.9. In some embodiments, the subject has an apo B/apo A-I ratio in a subject's blood plasma or blood serum of greater than 0.7. See Walldius, G. et al. J Intern Med. 2006 May; 259(5):493-519.

In some embodiments, a male subject having an apo B/apo A-I ratio in the male subject's blood plasma or blood serum of greater than 0.9 is considered for a primary prevention treatment. In some embodiments, a female subject having an apo B/apo A-I ratio in the female subject's blood plasma or blood serum of greater than 0.8 is considered for a primary prevention treatment. In some embodiments, the subject is a male subject and has an apo B/apo A-I ratio in the subject's blood plasma or blood serum of greater than 0.9. In some embodiments, the subject is a female subject and has an apo B/apo A-I ratio in the subject's blood plasma or blood serum of greater than 0.8. See Walldius, 2006.

In some embodiments, a male subject having an apo B/apo A-I ratio in the male subject's blood plasma or blood serum of greater than 0.7 is considered for a secondary prevention treatment. In some embodiments, a female subject having an apo B/apo A-I ratio in the female subject's blood plasma or blood serum of greater than 0.6 is considered for a secondary prevention treatment. In some embodiments, the subject is a male subject and has an apo B/apo A-I ratio in the subject's blood plasma or blood serum of greater than 0.7. In some embodiments, the subject is a female subject and has an apo B/apo A-I ratio in the subject's blood plasma or blood serum of greater than 0.6. See Walldius, 2006.

The present invention provides methods for treating a subject having or preventing a subject from having an abnormally low concentration in a subject's blood plasma or blood serum of high-density lipoprotein (HDL), comprising administering to a subject in need thereof an effective amount of the compound of the invention or the composition of the invention.

The present invention provides methods for treating a subject having or preventing a subject from having an abnormally reduced or deficient lipoprotein lipase concentration or activity in a subject's blood plasma or blood serum, comprising administering to a subject in need thereof an effective amount of a compound of the invention or a composition of the invention. In some embodiments, the reduced or deficient lipoprotein lipase level or activity is a result of a lipoprotein lipase mutation. In some embodiments, the reduced or deficient lipoprotein lipase level or activity is a result of a mutation in a gene encoding a lipoprotein lipase.

In some embodiments, the present invention provides methods for elevating an abnormally low concentration of lipoprotein lipase in a subject's blood plasma or blood serum, comprising administering to a subject in need thereof an effective amount of a compound of the invention or a composition of the invention. An “abnormally reduced concentration” of lipoprotein lipase in a subject's blood serum is less than 46 ng/mL. In some embodiments, the subject has an increased risk for future coronary artery disease. In some embodiments, the elevating is to a normal concentration. In some embodiments, the normal concentration of lipoprotein lipase in a subject's blood serum is greater than 46 ng/mL. In some embodiments, the present invention provides methods for treating a subject with lipoprotein lipase blood plasma or blood serum concentration of less than 46 ng/mL, comprising administering to a subject in need thereof an effective amount of a compound of the invention or a composition of the invention. See Rip, J. et al. Arterioscler Thromb Vasc Biol. 2006 March; 26(3):637-42. Epub 2005 Dec. 22.

The present invention provides methods for treating a subject having or preventing a subject from having an abnormally high concentration of lipoprotein-associated phospholipase A₂ (Lp-PLA₂) in a subject's blood plasma or blood serum, comprising administering to a subject in need thereof an effective amount of a compound of the invention or a composition of the invention. An “abnormally high concentration” of Lp-PLA₂ in a subject's blood plasma or blood serum is greater than 200 ng/mL. In some embodiments, the subject has a risk for developing cardiovascular disease. In some embodiments, the risk is high.

In some embodiments, the present invention provides methods for reducing an abnormally high concentration of Lp-PLA₂ in a subject's blood plasma or blood serum, comprising administering to a subject in need thereof an effective amount of a compound of the invention or a composition of the invention. In some embodiments, the reducing is to a normal concentration. In some embodiments, the normal concentration of Lp-PLA₂ in a subject's blood plasma or blood serum concentration is less than 200 ng/mL. In some embodiments, the present invention provides methods for treating a subject with Lp-PLA₂ blood plasma or blood serum concentration of greater than 200 ng/mL. See Davidson, M. H. et al, The American Journal of Cardiology, 2008, 101, S51.

The present invention provides methods for treating or preventing hypoalphalipoproteinemia, a lipoprotein abnormality associated with diabetes, a lipoprotein abnormality associated with obesity, a lipoprotein abnormality associated with Alzheimer's Disease, or familial combined hyperlipidemia, comprising administering to a subject in need thereof an effective amount of a compound of the invention or a composition of the invention.

The present invention provides methods for reducing in a subject's blood plasma or blood serum an abnormally high concentration of triglyceride, low-density lipoprotein cholesterol (LDL-C), very low-density lipoprotein cholesterol (VLDL-C), non-high-density lipoprotein cholesterol, (non-HDL-C), lipoprotein(a) (Lp(a)), apolipoprotein B, HDL/(VLDL+LDL) ratio, apolipoprotein C-II (apo C-II) or apolipoprotein C-III (apo C-III), comprising administering to a subject in need thereof an effective amount of a compound of the invention or a composition of the invention.

An “abnormally high concentration” of triglyceride in a subject's blood serum is greater than 150 mg/dL. In some embodiments, the reducing is to a normal concentration. In some embodiments, the normal concentration of triglyceride in a subject's blood serum is less than 150 mg/dL. In some embodiments, the present invention provides methods for reducing a subject's blood serum triglyceride concentration, where the subject has a blood serum triglyceride concentration greater than or equal to 200 mg/dL. In some embodiments, the present invention provides methods for reducing a subject's blood serum triglyceride concentration, where the subject has a blood serum triglyceride concentration greater than or equal to 500 mg/dL.

An “abnormally high concentration” of LDL-C in a subject's blood plasma or blood serum for primary prevention is greater than 100 mg/dL. An “abnormally high concentration” of LDL-C in a subject's blood plasma or blood serum for secondary prevention in a subject with risk factors is greater than 70 mg/dL. In some embodiments, the reducing is to a normal concentration. In some embodiments, the normal concentration of LDL-C in a subject's blood plasma or blood serum is less than 100 mg/dL, wherein the subject is being considered for primary prevention. In some embodiments, the normal concentration of LDL-C in a subject's blood plasma or blood serum is less than 70 mg/dL. In some embodiments, the subject has risk factors and is being considered for secondary prevention. See Walldius, 2006.

In some embodiments, an “abnormally high concentration” of LDL-C in a subject's blood plasma or blood serum is greater than 160 mg/dL. In some embodiments, the reducing is to a normal concentration. In some embodiments, the normal concentration of LDL-C in a subject's blood plasma or blood serum is less than 160 mg/dL. In some embodiments, the subject has 0-1 CHD risk factors. In some embodiments, the present invention provides methods for treating a subject with LDL-C blood plasma or blood serum concentration of greater than 160 mg/dL. In some embodiments, the present invention provides methods for treating a subject with LDL-C blood plasma or blood serum concentration of greater than 160 mg/dL. In some embodiments, the subject has 0-1 CHD risk factors. See Walldius, 2006.

In some embodiments, an “abnormally high concentration” of LDL-C in a subject's blood plasma or blood serum is greater than 130 mg/dL. In some embodiments, the subject has 2 or more CHD risk factors. In some embodiments, the reducing is to a normal concentration. In some embodiments, the normal concentration of LDL-C in a subject's blood plasma or blood serum is less than 130 mg/dL. In some embodiments, the subject has 2 or more CHD risk factors. In some embodiments, the present invention provides methods for treating a subject with LDL-C blood plasma or blood serum concentration of greater than 130 mg/dL. In some embodiments, the subject has 2 or more CHD risk factors. See Walldius, 2006.

In some embodiments, an “abnormally high concentration” of LDL-C in a subject's blood plasma or blood serum is greater than 100 mg/dL. In some embodiments, the subject has a CHD or a CHD risk equivalent. In some embodiments, the reducing is to a normal concentration. In some embodiments, the normal concentration of LDL-C in a subject's blood plasma or blood serum is less than 100 mg/dL. In some embodiments, the subject has a CHD or a CHD risk equivalent. In some embodiments, the present invention provides methods for treating a subject with LDL-C blood plasma or blood serum concentration of greaterthan 100 mg/dL. In some embodiments, the subject has a CHD or a CHD risk equivalent. See Walldius, 2006.

An “abnormally high concentration” of apo C-III concentration in a subject's blood plasma or blood serum is greater than 7.87 mg/dL. In some embodiments, the reducing is to a normal concentration. In some embodiments, the normal concentration of apo C-III concentration in a subject's blood plasma or blood serum is less than 7.87 mg/dL. In some embodiments, the present invention provides methods for treating a subject, where the subject has an apo C-III blood plasma or blood serum concentration greater than 8 mg/dL. In some embodiments, the present invention provides methods for treating a subject, where the subject has an apo C-III blood plasma or blood serum concentration greater than 7.9 mg/dL. In some embodiments, the present invention provides methods for treating a subject, where the subject has an apo C-III blood plasma or blood serum concentration greater than 7.87 mg/dL. In some embodiments, the abnormally high concentration of apo C-III is associated with high risk of coronary artery disease. See Capelleveen et al. Arterioscler Thromb Vasc Biol. 2017 June; 37(6): 1206-1212.

The present invention provides methods for reducing in a subject's blood plasma or blood serum an abnormally high LDL-C/HDL-C ratio, comprising administering to a subject in need thereof an effective amount of a compound of the invention or a composition of the invention.

In some embodiments, an “abnormally high ratio” of LDL-C/HDL-C in a male subject's blood plasma or blood serum for primary prevention is greater than 3.0. In some embodiments, the reducing is to a normal ratio. In some embodiments, the normal ratio of LDL-C/HDL-C in a male subject's blood plasma or blood serum is less than 3.0, wherein the subject is being considered for primary prevention. In some embodiments, the present invention provides methods for treating a male subject with an LDL-C/HDL-C ratio in a subject's blood plasma or blood serum of greater than 3.0. In some embodiment, the me method is for primary prevention.

In some embodiments, an “abnormally high ratio” of LDL-C/HDL-C in a female subject's blood plasma or blood serum for primary prevention is greater than 2.5. In some embodiments, the reducing is to a normal ratio. In some embodiments, the normal ratio of LDL-C/HDL-C in a female subject's blood plasma or blood serum is less than 2.5, wherein the subject is being considered for primary prevention. In some embodiments, the present invention provides methods for treating a female subject with an LDL-C/HDL-C ratio in a subject's blood plasma or blood serum of greater than 2.5. In some embodiment, the me method is for primary prevention. See Walldius, 2006.

In some embodiments, an “abnormally high ratio” of LDL-C/HDL-C in a male subject's blood plasma or blood serum for secondary prevention is greater than 2.5. In some embodiments, the reducing is to a normal ratio. In some embodiments, the normal ratio of LDL-C/HDL-C in a male subject's blood plasma or blood serum is less than 2.5, wherein the subject is being considered for secondary prevention. In some embodiments, the present invention provides methods for treating a male subject with an LDL-C/HDL-C ratio in a subjects blood plasma or blood serum of greater than 2.5. In some embodiment, the me method is for secondary prevention. See Walldius, 2006.

In some embodiments, an “abnormally high ratio” of LDL-C/HDL-C in a female subject's blood plasma or blood serum for secondary prevention is greater than 2.0. In some embodiments, the reducing is to a normal ratio. In some embodiments, the normal ratio of LDL-C/HDL-C in a female subject's blood plasma or blood serum is less than 2.0, wherein the subject is being considered for secondary prevention. In some embodiments, the present invention provides methods for treating a female subject with an LDL-C/HDL-C ratio in a subject's blood plasma or blood serum of greater than 2.0. In some embodiment, the me method is for secondary prevention. See Walldius, 2006.

The present invention provides methods for reducing in a subject's blood plasma or blood serum an abnormally high concentration of non-HDL-C, comprising administering to a subject in need thereof an effective amount of a compound of the invention or a composition of the invention.

In some embodiments, an “abnormally high concentration” of non-HDL-C in a subject's blood plasma or blood serum is greater than 190 mg/dL. In some embodiments, the reducing is to a normal concentration. In some embodiments, the normal concentration of non-HDL-C in a subject's blood plasma or blood serum is less than 190 mg/dL. In some embodiments, the subject has 0-1 CHD risk factors. In some embodiments, the present invention provides methods for reducing a subject's non-HDL-C concentration in the subject's blood plasma or blood serum, wherein the non-HDL-C concentration is greater than 190 mg/dL. In some embodiments, the subject has 0-1 CHD risk factors. See Walldius, 2006.

In some embodiments, an “abnormally high concentration” of non-HDL-C in a subject's blood plasma or blood serum is greater than 160 mg/dL. In some embodiments, the subject has 2 or more CHD risk factors. In some embodiments, the reducing is to a normal concentration. In some embodiments, the normal concentration of non-HDL-C in a subject's blood plasma or blood serum is less than 160 mg/dL. In some embodiment, the subject has 2 or more CHD risk factors. In some embodiments, the present invention provides methods for reducing a subject's non-HDL-C concentration in the subject's blood plasma or blood serum, wherein the subject's non-HDL-C concentration is greater than 160 mg/dL. In some embodiments, the subject has 2 or more CHD risk factors. See Walldius, 2006.

In some embodiments, an “abnormally high concentration” of non-HDL-C in a subject's blood plasma or blood serum is greater than 130 mg/dL. In some embodiments, the subject has a CHD or a CHD risk equivalent. In some embodiments, the reducing is to a normal concentration. In some embodiments, the normal concentration of non-HDL-C in a subject's blood plasma or blood serum is less than 130 mg/dL. In some embodiments, the subject has a CHD or a CHD risk equivalent. In some embodiments, the present invention provides methods for reducing a subject's non-HDL-C concentration in the subject's blood plasma or blood serum, wherein the subject's non-HDL-C concentration is greater than 130 mg/dL. In some embodiments, the subject has a CHD or a CHD risk equivalent. See Walldius, 2006.

The present invention provides methods for elevating in a subject's blood plasma or blood serum an abnormally low concentration of a high-density lipoprotein (HDL)-associated protein, HDL-cholesterol (HDL-C), apolipoprotein A-I, orapolipoprotein E, comprising administering to a subject in need thereof an effective amount of a compound of the invention or a composition of the invention. In some embodiments, the HDL-associated protein is apolipoprotein A-I (apo A-I), apolipoprotein A-II (apo A-II), apolipoprotein A-IV (apo A-IV) or apolipoprotein E (apo E).

In some embodiments, an “abnormally low concentration” of HDL-C in a subject's blood plasma or blood serum is less than 40 mg/dL. In some embodiments, the elevating is to a normal concentration. In some embodiments, the normal concentration of HDL-C in a subject's blood plasma or blood serum is greater than 40 mg/dL. In some embodiments, the present invention provides methods for elevating HDL-C concentration in a subject's blood plasma or blood serum, where the subject's HDL-C concentration is less than 40 mg/dL. In some embodiments, the subject is a male subject. In some embodiments, the subject is a female subject.

In some embodiments, an “abnormally low concentration” of HDL-C in a male subject's blood plasma or blood serum is less than 45 mg/dL. In some embodiments, the normal concentration of HDL-C in a male subject's blood plasma or blood serum is greater than 45 mg/dL. In some embodiments, the present invention provides methods for elevating HDL-C concentration in a male subject's blood plasma or blood serum, where the subject's HDL-C concentration is less than 45 mg/dL.

In some embodiments, an “abnormally low concentration” of HDL-C in a female subject's blood plasma or blood serum is less than 50 mg/dL. In some embodiments, the normal concentration of HDL-C in a female subject's blood plasma or blood serum is greater than 50 mg/dL. In some embodiments, the normal concentration of HDL-C in a female subject's blood plasma or blood serum is greater than 55 mg/dL. In some embodiments, the present invention provides methods for elevating HDL-C concentration in a female subject's blood plasma or blood serum, where the subject's HDL-C concentration is less than 50 mg/dL. In some embodiments, the present invention provides methods for elevating HDL-C concentration in a female subject's blood plasma or blood serum where the subject's HDL-C concentration is lower than 55 mg/dL.

In some embodiments, the present invention provides methods for elevating HDL-C concentration in a subject's blood plasma or blood serum to 45 mg/dL or greater, where the subject's HDL-C concentration in a subject's blood plasma or blood serum is less than 40 mg/dL. In some embodiments, the present invention provides methods for elevating HDL-C concentration in a subject's blood plasma or blood serum to 50 mg/dL or greater, where the subject's HDL-C concentration in a subject's blood plasma or blood serum is less than 40 mg/dL. In some embodiments, the present invention provides methods for elevating HDL-C concentration in a subject's blood plasma or blood serum to 55 mg/dL or greater, where the subject's HDL-C concentration in a subject's blood plasma or blood serum is less than 40 mg/dL.

In some embodiments, the present invention provides methods for elevating HDL-C concentration in a male subject's blood plasma or blood serum to 50 mg/dL or greater, where the subject's HDL-C concentration in a subject's blood plasma or blood serum is less than 45 mg/dL. In some embodiments, the present invention provides methods for elevating HDL-C concentration in a female subject's blood plasma or blood serum to 50 mg/dL or greater, where the subject's HDL-C concentration in a subject's blood plasma or blood serum is less than 50 mg/dL. In some embodiments, the present invention provides methods for elevating HDL-C concentration in a female subject's blood plasma or blood serum to 55 mg/dL or greater, where the subject's HDL-C concentration in a subject's blood plasma or blood serum is less than 50 mg/dL.

The present invention provides methods for promoting clearance of triglyceride from a subject's blood plasma or blood serum, comprising administering to a subject in need thereof an effective amount of the compound of the invention or the composition of the invention.

The present invention provides methods for increasing an abnormally low glucose metabolism or increasing an abnormally low lipid metabolism in a subject, comprising administering to a subject in need thereof an effective amount of a compound of the invention or a composition of the invention. In some embodiments, the method for increasing an abnormally low glucose metabolism increases insulin sensitivity or oxygen consumption in a subject. In some embodiments, the method for increasing an abnormally low glucose metabolism reduces blood insulin, blood glucose, or glycated hemoglobin in a subject's blood plasma or blood serum. In some embodiments, the methods for increasing an abnormally low lipid metabolism reduces a concentration of LDL or free triglyceride in a subject's blood plasma or blood serum, or inhibits saponified or non-saponified fatty acid synthesis.

In some embodiments, a subject with abnormally low glucose metabolism has an abnormally high concentration of glucose or hemoglobin (Hb) in the subject's blood plasma or blood serum. In some embodiments, the present invention provides methods for reducing an abnormally high concentration of glucose or hemoglobin in a subject's blood plasma or blood serum. In some embodiments, the method increases abnormally low glucose metabolism. An “abnormally high concentration” of glucose in a subject's blood plasma or blood serum in a two-hour GTT is greater than 7.8 mmol/L (140 mg/dL). In some embodiments, reducing is to a normal concentration. In some embodiments, the normal concentration of glucose in a subject's blood plasma or blood serum in a two-hour GTT is less than 7.8 mmol/L (140 mg/dL). In some embodiments, the present invention provides methods for increasing abnormally low glucose metabolism in a subject's blood plasma or blood serum, where the subject's glucose concentration in the subject's blood plasma or blood serum in a two-hour GTT is greater than 7.8 mmol/L (140 mg/dL). In some embodiments, the present invention provides methods for increasing abnormally low glucose metabolism in a subject, where the subject's glucose concentration in the subject's blood plasma or blood serum in a two-hour GTT ranges from 7.8 mmol/L (140 mg/dL) to 11.1 mmol/L (200 mg/dL). In some embodiments, the present invention provides methods for increasing abnormally low glucose metabolism in a subject, where the subject's glucose concentration in the subject's blood plasma or blood serum in a two-hour GTT is above 11.1 mmol/L (200 mg/dL).

In some embodiments, the present invention provides methods for increasing abnormally low glucose metabolism in a subject, where the subject has impaired glucose tolerance. In some embodiments, the present invention provides methods for increasing abnormally low glucose metabolism in a subject, where the subject has diabetes. In some embodiments, the present invention provides methods for increasing abnormally low glucose metabolism in a subject, where the subject has gestational diabetes.

A low lipid metabolism can be characterized by dyslipidemia (using the LDL-C, TGs, non-HDL-chol, apo B, apo C-III or apo C-II values) but also with elevated concentration of transaminases. In some embodiments, the subject having a low lipid metabolism also has dyslipidemia.

The present invention provides methods for treating or preventing a symptom of a disease selected from inflammation, systemic lupus erythematosus, lupus nephritis, or arthritis, comprising administering to a subject in need thereof an effective amount of a compound of the invention or a composition of the invention. In some embodiments, the arthritis is adjuvant arthritis or type II collagen-induced arthritis. In some embodiments, the symptom is nephritis, kidney failure, or kidney function reduction. In some embodiments, the kidney function reduction requires renal dialysis.

The present invention provides methods for reducing fat content of meat in livestock, comprising administering to livestock an effective amount of a compound of the invention or a composition of the invention.

The present invention provides methods for reducing cholesterol content of a fowl egg, comprising administering to a fowl species an effective amount of a compound of the invention or a composition of the invention.

In some embodiments, the methods of the invention provide administering a dosage form of the compounds of the invention or the compositions of the invention hourly, daily, weekly, or monthly. The dosage forms and formulations of the present invention may be administered three times a day, twice a day or once a day. The dosage forms of the present invention can be administered with food or without food. An appropriate length of the treatment, dosages, and route of administration can be determined and/or adjusted by a physician.

In some embodiments of the methods of the invention, the subject is a human subject.

### EXAMPLES

### Example 1. Synthesis of Compound I

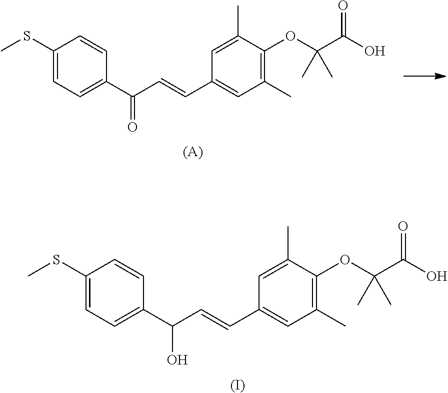 [SMILES: CSc1ccc(C(=O)/C=C/c2cc(C)c(OC(C)(C)C(=O)O)c(C)c2)cc1 ; "(A)" ; CSc1ccc(C(O)/C=C/c2cc(C)c(OC(C)(C)C(=O)O)c(C)c2)cc1 ; "(I)"]

(Z)-2-(2,6-dimethyl-4-(3-(4-(methylthio)phenyl)-3-oxoprop-1-en-1-yl)phenoxy)-2-methylpropanoic acid (“Compound A”) is synthesized according to US2006/0142611. Compound A is then reduced using a reducing agent, such as NaBH₄, optionally in the presence of a Lewis acid, such as CeCl₃, or in an alcohol solvent, such as methanol, to provide Compound I racemate. Compound I racemate is then resolved to provide its (S)- and (R)-enantiomers, each being substantially free of its corresponding opposite enantiomer, using a chiral high performance liquid chromatography.

### Example 2. Synthesis of Compound II

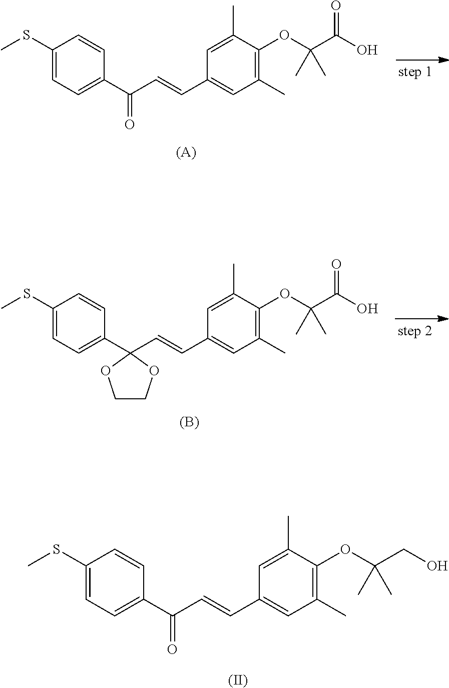 [SMILES: CSc1ccc(C(=O)/C=C/c2cc(C)c(OC(C)(C)C(=O)O)c(C)c2)cc1 ; "(A)" ; CSc1ccc(C2(/C=C/c3cc(C)c(OC(C)(C)C(=O)O)c(C)c3)OCCO2)cc1 ; "(B)" ; CSc1ccc(C(=O)/C=C/c2cc(C)c(OC(C)(C)CO)c(C)c2)cc1 ; "(II)"]

Acetal Compound B is synthesized from Compound A and ethylene glycol in the presence of catalytic acid, such as 0.1 M HCl. See, e.g., Dong, J-L., et al. ACS Omega, 2018, 3, 4974 for acetal formation specifically on □□□-unsaturated ketones. The carboxyl group of Compound B is subsequently reduced to a hydroxymethyl group using a reducing reagent, such as LiAlH₄, followed by an aqueous acid workup, which removes the acetal to provide Compound II.

### Example 3. Synthesis of Compound III

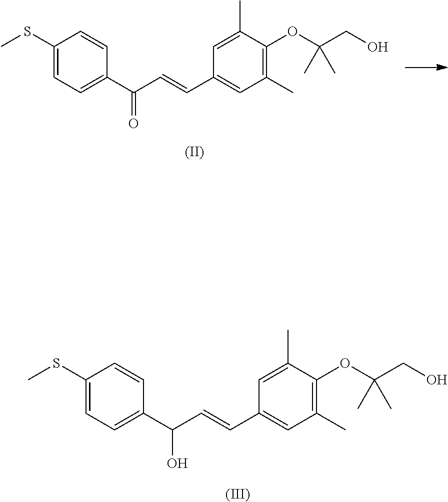

Compound II is reduced using a reducing agent, such as NaBH₄, optionally in the presence of a Lewis acid, such as CeCl₃, or in an alcohol solvent, such as methanol, to provide Compound III racemate. Compound III racemate is then resolved to provide its (S)- and (R)-enantiomers, each being substantially free of its corresponding opposite enantiomer, using a chiral high performance liquid chromatography.

### Example 4. Hepatic and Peripheral Insulin Sensitive Effects in Rats Fed a High Fat/Medium Fructose Diet (Western Diet)

After 8 weeks of high fat diet, rats are treated for 5 weeks with pioglitazone (10 mg/kg), metformin (50 mg/kg), an illustrative compound of the invention (3 and 10 mg/kg), alone or in association with metformin (50 mg/kg), and GW501516 (2-[2-methyl-4-[[4-methyl-2-[4-(trifluoromethyl)phenyl]-1,3-thiazol-5-yl]methylsulfanyl]phenoxy]acetic acid) (10 mg/kg; ligand/GSK). Body weight can be decreased by metformin and this effect can be emphasized when it is associated with the illustrative compound of the invention in a dose of 10 mg/kg. After 17 days of treatment, rats are fasted for 4 hours and blood is sampled.

The glucose tolerance is assessed after 21 days of treatment by an oral glucose load performed after 6 hours of fasting.

After 5 weeks of treatment, an euglycemic-hyperinsulinemic clamp procedure is performed in awake rats. Two doses of insulin are infused with 3H-glucose: 0.2 U/kg/h to assess an effect on hepatic glucose production (HGP) and 0.8 U/kg/h to inhibit HGP and then to assess an effect on whole body glucose utilization.

### Animal Model

Male Sprague Dawley (SD) rats (250-275 g) are first fed with the western diet during 8 weeks for inducing insulin resistance. The first set of rats undergoes an oral glucose tolerance test (OGTT) after 3 weeks of treatment (half rats/group of the clamp arm) and the second set also undergoes an OGTT after 3 weeks of treatment (half rats/group of clamp arm) and the histology study (3 rats/group).

Rats of each set are screened and randomized into the several groups according to their fasted (4 h) plasma glucose, insulin levels (for homeostatic model assessment of insulin resistance (HOMA-IR) calculation) and body weight. Rats that do not respond to the western diet are excluded from the study.

Homogenous mild obese and insulin resistant rats are allocated into the different treatment groups and western diet is continued until the end of the experiment. Given that, the 2 series started with a gap of one week.

### Treatments

Dosage regimen: Rats are treated once daily via the oral route, in the morning. The duration of the treatment is between 5 and 5.5 weeks.

### Test Groups

Group 1: vehicle (n=12)

Group 2: pioglitazone, 10 mg/kg (n=12)

Group 3: metformin, 50 mg/Kg (n=12)

Group 4: an illustrative compound of the invention, 3 mg/Kg (n=12)

Group 5: an illustrative compound of the invention, 10 mg/Kg (n=12)

Group 6: an illustrative compound of the invention, 3 mg/Kg+metformin 50 mg/kg (n=12)

Group 7: an illustrative compound of the invention, 10 mg/Kg+metformin 50 mg/kg (n=12)

Group 8: GW501216, 10 mg/Kg (n=12)

### Fasting Conditions

Fasting conditions for the OGTT and the clamp procedure: food is removed from the cage and litter is changed just before lights-off (between 7:30 and 8:00 am). Then each experiment starts after about 6 hours of fasting (between 1:30 and 2:00 μm).

Fasting conditions for plasma parameters: food is removed from the cage and litter is changed 4 hours after lights-off (between 11:30 am and noon). Then blood collection starts after 4 hours of fasting (at 4:00 μm).

Oral Glucose Tolerance Test

After 3 weeks of treatment, rats are fasted for 6 hours and an oral glucose load (2.5 g/kg body weight) is performed. Blood glucose is measured 30 minutes before glucose load and at 0, 15, 30, 60, 90 and 120 minutes.

Euglycemic-Hyperinsulinemic 2 Steps Clamp with 3H-Glucose

After 4 weeks of treatment, a catheter is implanted into the femoral vein under isoflurane anesthesia and a period of 5-6 days is respected for recovery. Rats that do not recover body weight are excluded from the study. The accepted body weight loss estimated on the day of perfusion is fixed at 5% in groups where treatments should not affect body weight (as seen during body weight follow-up) (vehicle, pioglitazone and the illustrative compound of the invention, mg/kg groups) and at 10% where treatments decreases it (the 5 other groups). The morning of the clamp procedure, rats are treated and fasted for 6 hours prior to tracer, glucose and insulin infusions. The beginning of the clamp procedure is performed in the middle of the dark cycle.

The euglycemic-hyperinsulinemic 2 steps insulin clamp is performed using 0.2 U/kg/h and 0.8 U/kg/h insulin infusion associated with 3H-glucose infusion. Thereafter the following parameters are assessed:

Whole body glucose utilization

Hepatic glucose production

Glucose infusion rate

Whole body glycogen and glycolytic rates

In Vivo Glucose Utilization Rate

During the clamp procedure, a bolus (30 μCi) of D-[3-3H]glucose is first injected followed by 4 μCi/min/kg infusion rate during all the experiment to ensure a detectable plasma D[3-3H]glucose enrichment. A bolus of insulin (100 mU) is first injected, and insulin is then infused at a rate of 0.2 U/kg/h for the first 2 hours and 0.8 U/kg/h from 120 minutes to 210 minutes.

Throughout the infusion, glycaemia is assessed with a blood glucose meter from the tip of the tail vein when needed. Euglycemia is maintained by periodically (every 10 minutes) adjusting a variable infusion of 30% glucose. Plasma glucose concentrations and D-[3-3H]glucose specific activity are determined during stable phase in 10 μl of blood sampled from the tip of the tail vein every 20 minutes from 60 to 120 minutes during the first step and from 150 to 210 minutes during the second step.

For glucose turnover measurements, D-[3-3H]glucose enrichments are determined from total blood after deproteinization by a Zn(OH)₂ precipitate. Briefly, an aliquot of the supernatant is evaporated to dryness to determine the radioactivity corresponding to D-3-3H. In a second aliquot of the same supernatant, glucose concentration is assessed by the glucose oxidase method (Biomérieux, France).

### Calculation

Calculations for glucose turnover measurements are made from parameters obtained during the infusions in steady-state condition (60-120 and 160-210 minutes). Briefly, the D-[3-3H]glucose specific activity is calculated by dividing the D-[3-3H]glucose enrichment by the plasma glucose concentration. The whole body glucose turnover rate is calculated by dividing the rate of D[3-3H]glucose by the D-[3-3H]glucose plasma specific activity. For each rat, the mean values are calculated and averaged with values from rats of the same group. The whole body glycolysis rate are measured by assessing the amount of tritiated water accumulated in the blood during the 3H-glucose infusion and the whole body glycogen synthesis rate are calculated by the difference between the whole body glucose turnover and the whole body glycolysis rate.

### Blood, Tissue Collection and Biochemistry

At the end of the clamp procedure (9.5 hours fasting), perirenal, retroperitoneal, epididymal and inguinal fat pads and liver weights are recorded. Blood is collected from cardiac puncture and plasmas are stored at −80° C. for further assays if needed (depending on the radioactive state of samples). Liver triglyceride content is assessed (enzymatic-colorimetric method) as well as liver and adipose tissue TNF-α content (enzyme-linked immunosorbent assay (ELISA) method).

See US 2011/0092517, which is hereby incorporated by reference in its entirety.

### Example 5. Mouse and Rat PPAR Transcriptional Activation in Cell-Based Transactivation Assay

Assays with GAL4-PPAR Chimera Receptors

### Receptor Expression Plasmids

An established chimeric receptor system (Lehmann J M et. al. J Biol Chem. 1997; 272(6):3406-10) is utilized to allow comparison of the relative transcriptional activity of the receptor subtypes. The mammalian expression vectors pSG5-GAL4-PPARα, pSG5-GAL4-PPARγ and pSG5-GAL4-PPARδ from Homo sapiens (hs) (NM_006238, NM_015869, NM_001001928), Macaca mulatta (mm) (NM_001033029, XM_001116676, NM_001032860), Mus musculus (m) (NM_011144, U10375, NM_011146) and Rattus norvegicus (r) (NM_013141, NM_013196, NM_013124), which express the ligand binding domains (LBDs) of human PPARRα (amino acids 167-468 for hs and m, amino acids 167-467 for mm), PPARγ1 (amino acids 176-477 for hs, amino acids 204-505 for m and mm from PPARγ) and PPARδ (amino acids 139-441 for hs and mm, amino acids 139-440 for m) each fused to the yeast transcription factor GAL4 DNA binding domain (amino acid 1-147) and the human glucocorticoid receptor (amino acids 1-76), are cloned.

Reporter plasmid: MH100×4-tk-luc (Forman B M et al. Cell. 1995 81(4):541-50) is used as the reporter plasmid.

### Transient Transfection Assays

The African green monkey kidney cell line, CV-1 is used for the transfection assays. CV-1 cells are seeded in 24-well plates at 0.5×10⁵ cells per well and are cultured for 24 hours. Transfection mixtures for chimera receptors contain 30 ng of receptor expression plasmid, 120 ng of the reporter plasmid, 350 ng of pCMX-β-galactosidase (βGAL) expression plasmid as a control for transfection efficiency, 250 ng of pGEM4 carrier plasmid and 2 μL of a lipofection reagent (Lipofectamine 2000, Invitrogen). These mixtures are added to cells and incubated for 5 hours according to the manufacturer's instructions. After the transfection, cells are incubated for an additional 40 hours in the presence of the illustrative compounds of the invention or each reference compound at different concentrations. Cell lysates are prepared with a lysis buffer (Passive Lysis Buffer, Promega) and used in the luciferase and βGAL assays. The luciferase and βGAL activity are measured with the Luciferase assay system (E4030, Promega) and with the βGAL enzyme assay system (E2000, Promega). Assays are performed in triplicate for GAL4-chimeras. Experiments are repeated at least three times.

### Calculation of Relative PPAR Transactivation Activities

Each point of a relative PPAR transcriptional activity to maximal activity is calculated based on the values bellow:

Luciferase activity of cells treated with a positive control (10-5 M GW-590735 for hPPARα, 3×10-5 M rosiglitazone maleate for hPPARγ assays and 10-5 M GW-501516 for hPPARδ) as the maximal activity, and luciferase activity of cells treated with 0.1% of DMSO as the minimum activity.

### Calculation of EC₅₀ Values.

EC₅₀ values defined as the concentration of the illustrative compound of the invention and GW-501516 to produce 50% of maximal reporter activity re calculated with Prism software (Graphpad Software).

Experiments with 10% Serum Vs 0.1% Serum.

Experiments at 2 serum concentrations: 10% and 0.1%. The EC50 are calculated as previously described.

First, the assay is set up and the GAL4-chimeras/reporter plasmids are validated using Homo-sapiens sequences of the different PPAR. The experiments are conducted at 10% and 0.1% serum with GW501516 (PPARδ agonist).

Plasmid Reference is as indicated in US 2011/0092517, which is hereby incorporated by reference in its entirety.

The disclosures of all publications, patents, patent applications and published patent applications referred to herein by an identifying citation are hereby incorporated herein by reference in their entirety.

What is claimed is:

1. A compound of Formula (A): 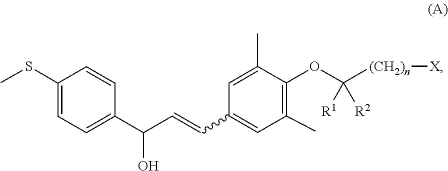
or a pharmaceutically acceptable salt, solvate, ester, amide, or prodrug thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COOH, —COH, —COOR³, —COOCH₂CONR⁴R⁶, —SO₃H, 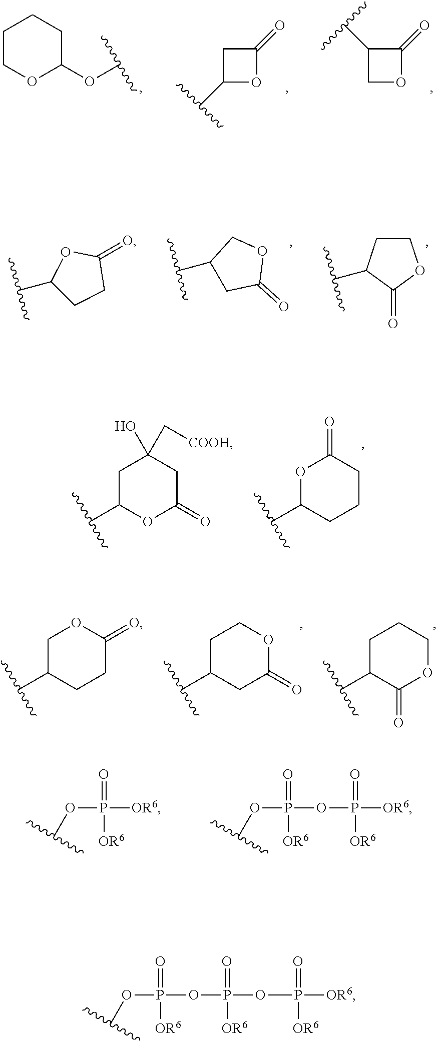 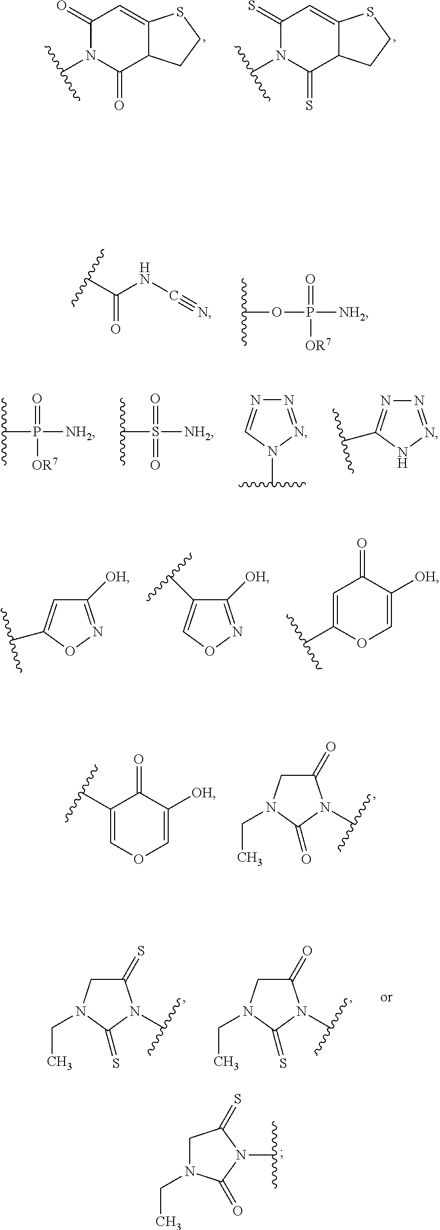
R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

2. The compound or pharmaceutically acceptable salt, solvate, ester, amide, or prodrug of the compound of claim 1, wherein the compound is a racemate or a mixture of enantiomers or diastereomers.

3. The compound or pharmaceutically acceptable salt, solvate, ester, amide, or prodrug of the compound of claim 1, wherein the compound has an hydroxyl-bearing allylic carbon atom having an (R)-stereochemistry and has the structure

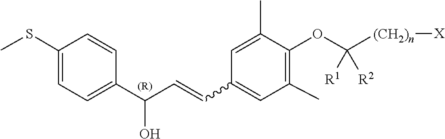

4. The compound or pharmaceutically acceptable salt, solvate, ester, amide, or prodrug of the compound of claim 1, wherein the compound has an hydroxyl-bearing allylic carbon atom having an (S)-stereochemistry and has the structure OH

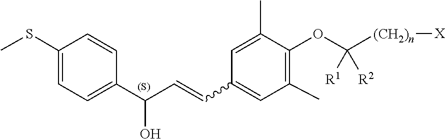

5. The compound or pharmaceutically acceptable salt, solvate, ester, amide, or prodrug of the compound of claim 3 or 4, wherein the compound is substantially free of its corresponding opposite stereoisomer.

6. The compound or pharmaceutically acceptable salt, solvate, ester, amide, or prodrug of the compound of any one of claims 3-5, wherein the compound has an olefin isomer configuration of (Z) or (E).

7. The compound or pharmaceutically acceptable salt, solvate, ester, amide, or prodrug of the compound of claim 1, wherein the compound is a (Z)-isomer and has the structure

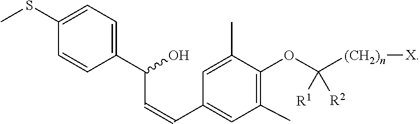

8. The compound or pharmaceutically acceptable salt, solvate, ester, amide, or prodrug of the compound of claim 1, wherein the compound is a (E)-isomer and has the structure

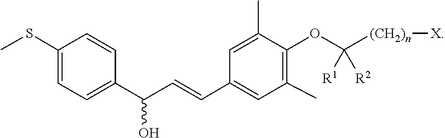

9. The compound or pharmaceutically acceptable salt, solvate, ester, amide, or prodrug of the compound of claim 7 or 8, wherein the compound is substantially free of its corresponding other olefin configuration.

10. The compound or pharmaceutically acceptable salt, solvate, ester, amide, or prodrug of the compound of any one of claims 7-9, wherein the compound has an hydroxyl-bearing allylic carbon atom having an (R)- or an (S)-stereochemistry.

11. The compound or pharmaceutically acceptable salt, solvate, ester, amide, or prodrug of any one of the compound of claims 1-10, wherein each R¹ and R² is independently —C₁-C₃ alkyl.

12. The compound or pharmaceutically acceptable salt, solvate, ester, amide, or prodrug of any one of the compound of claims 1-10, wherein each R¹ and R² is independently methyl.

13. The compound or pharmaceutically acceptable salt, solvate, ester, amide, or prodrug of any one of the compound of claims 1-12, wherein X is —CH₂OH, —COOH, —COH, —COOR³, or —COOCH₂CONR⁴R⁵.

14. The compound or pharmaceutically acceptable salt, solvate, ester, amide, or prodrug of any one of the compound of claims 1-12, wherein X is —CH₂OH or —COOH.

15. The compound or pharmaceutically acceptable salt, solvate, ester, amide, or prodrug of any one of the compound of claims 1-14, wherein n is 0 or 1.

16. A compound of Formula (B): 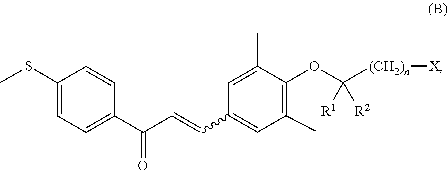
or a pharmaceutically acceptable salt, solvate, ester, amide, or prodrug thereof, wherein:
each R¹ and R² is independently —C₁-C₆ alkyl, —C₂-C₆ alkenyl, —C₂-C₆ alkynyl, phenyl, or benzyl; or alternatively, R¹ and R² together with the carbon atom to which R¹ and R² are attached form a C₃-C₇ cycloalkyl group;
X is —CH₂OH, —COH, —COOCH₂CONR⁴R⁵, —SO₃H, 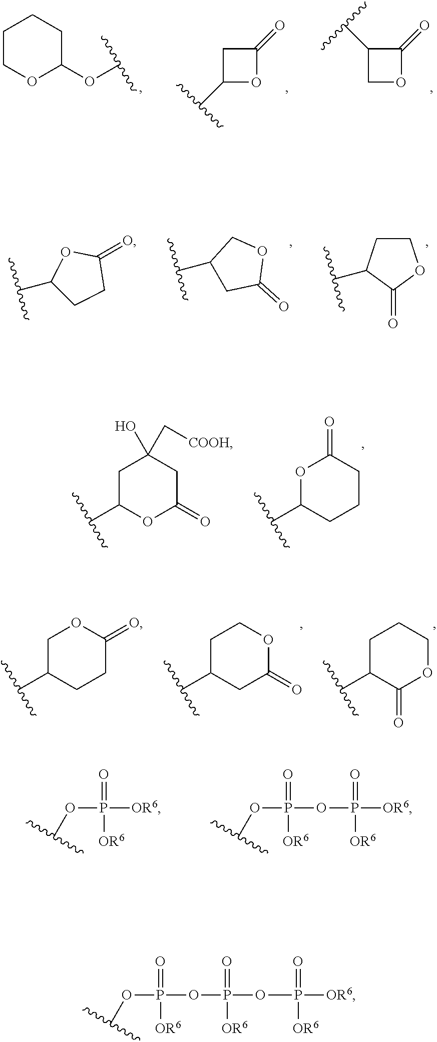 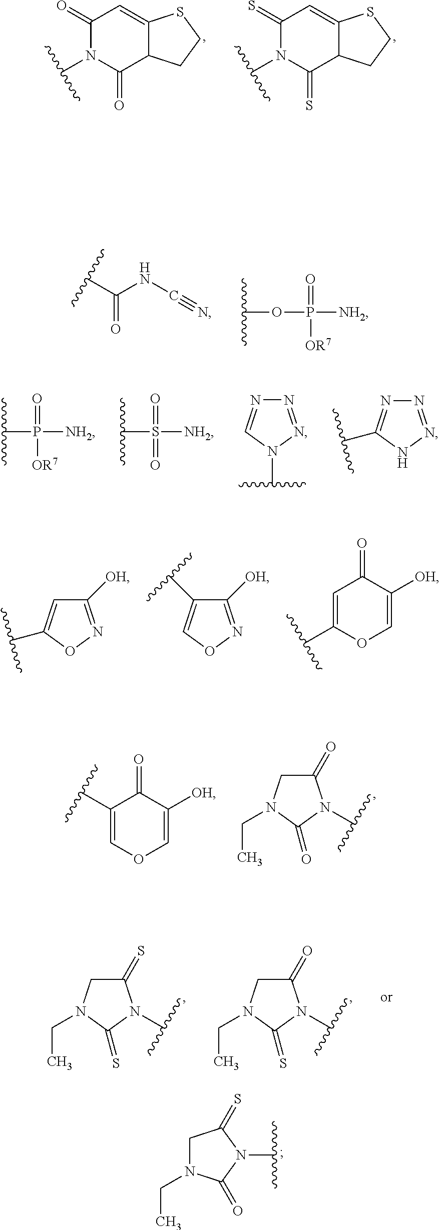
R³ is —C₁-C₆ alkyl, —C₂-C₆ alkenyl, —C₂-C₆ alkynyl, phenyl, or benzyl;
each R⁴ and R⁵ is independently alkyl, aryl, or heteroaryl; or alternatively, R⁴ and R⁵ together with the carbon atom to which R⁴ and R⁵ are attached form a heterocycle;
each R⁶ and R⁷ is independently H, —C₁-C₆ alkyl, —C₂-C₆ alkenyl, or —C₂-C₆ alkynyl; and
n is 0, 1, 2, 3, or 4.

17. The compound or pharmaceutically acceptable salt, solvate, ester, amide, or prodrug of the compound of claim 1, wherein the compound is a mixture of (Z)- and (E)-isomers.

18. The compound or pharmaceutically acceptable salt, solvate, ester, amide, or prodrug of the compound of claim 16, wherein the compound is a (Z)-isomer and has the structure

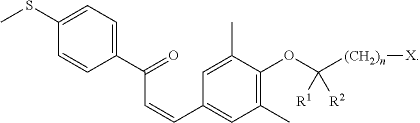

19. The compound or pharmaceutically acceptable salt, solvate, ester, amide, or prodrug of the compound of claim 16, wherein the compound is a (E)-isomer and has the structure

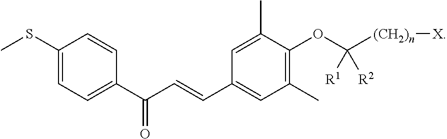

20. The compound or pharmaceutically acceptable salt, solvate, ester, amide, or prodrug of the compound of claim 18 or 19, wherein the compound is substantially free of its corresponding other olefin configuration.

21. The compound or pharmaceutically acceptable salt, solvate, ester, amide, or prodrug of the compound of any one of claims 16-20, wherein each R¹ and R² is independently —C₁-C₃ alkyl.

22. The compound or pharmaceutically acceptable salt, solvate, ester, amide, or prodrug of any one of claims 16-20, wherein each R¹ and R² is independently methyl.

23. The compound or pharmaceutically acceptable salt, solvate, ester, amide, or prodrug of any one of the compound of claims 16-22, wherein X is —CH₂OH, —COH, or —COOCH₂CONR⁴R⁵.

24. The compound or pharmaceutically acceptable salt, solvate, ester, amide, or prodrug of any one of the compound of claims 16-22, wherein X is —CH₂OH.

25. The compound or pharmaceutically acceptable salt, solvate, ester, amide, or prodrug of any one of the compound of claims 16-24, wherein n is 0 or 1.

26. A compound having the structure: 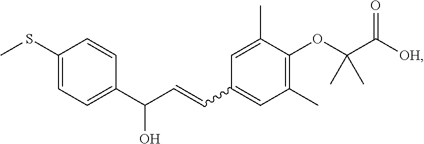 [SMILES: CSc1ccc(C(O)C=Cc2cc(C)c(OC(C)(C)C(=O)O)c(C)c2)cc1]
or a pharmaceutically acceptable salt, solvate, ester, amide, or prodrug thereof.

27. The compound or pharmaceutically acceptable salt, solvate, ester, amide, or prodrug of the compound of claim 26, wherein the compound is a racemate or a mixture of enantiomers.

28. The compound or pharmaceutically acceptable salt, solvate, ester, amide, or prodrug of the compound of claim 26, wherein the compound has an hydroxyl-bearing allylic carbon atom having an (R)-stereochemistry and has the structure

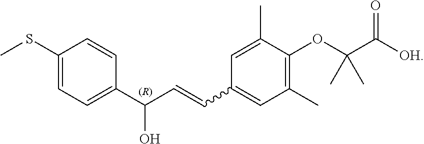

29. The compound or pharmaceutically acceptable salt, solvate, ester, amide, or prodrug of the compound of claim 26, wherein the compound has an hydroxyl-bearing allylic carbon atom having an (S)-stereochemistry and has the structure

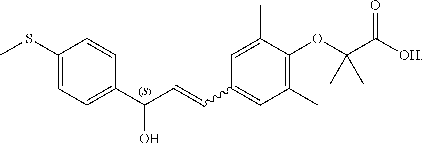

30. The compound or pharmaceutically acceptable salt, solvate, ester, amide, or prodrug of the compound of claim 28 or 29, wherein the compound is substantially free of its corresponding opposite enantiomer.

31. The compound or pharmaceutically acceptable salt, solvate, ester, amide, or prodrug of the compound of any one of claims 28-30, wherein the compound has an olefin isomer configuration of (Z) or (E).

32. The compound or pharmaceutically acceptable salt, solvate, ester, amide, or prodrug of the compound of claim 26, wherein the compound is a (Z)-isomer and has the structure

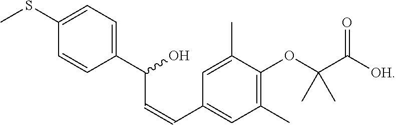 [SMILES: CSc1ccc(C(O)/C=C\c2cc(C)c(OC(C)(C)C(=O)O)c(C)c2)cc1]

33. The compound or pharmaceutically acceptable salt, solvate, ester, amide, or prodrug of the compound of claim 26, wherein the compound is a (E)-isomer and has the structure

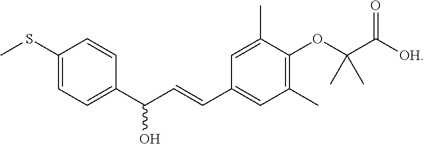 [SMILES: CSc1ccc(C(O)/C=C/c2cc(C)c(OC(C)(C)C(=O)O)c(C)c2)cc1]

34. The compound or pharmaceutically acceptable salt, solvate, ester, amide, or prodrug of the compound of claim 32 or 33, wherein the compound is substantially free of its corresponding other olefin configuration.

35. The compound or pharmaceutically acceptable salt, solvate, ester, amide, or prodrug of the compound of any one of claims 32-34, wherein the compound has an hydroxyl-bearing allylic carbon atom having an (R)- or an (S)-stereochemistry.

36. A compound having the structure: 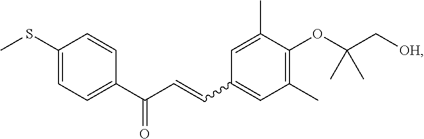 [SMILES: CSc1ccc(C(=O)C=Cc2cc(C)c(OC(C)(C)CO)c(C)c2)cc1]
or a pharmaceutically acceptable salt, solvate, ester, amide, or prodrug thereof.

37. The compound or pharmaceutically acceptable salt, solvate, ester, amide, or prodrug of the compound of claim 36, wherein the compound is a mixture of (Z)- and (E)-isomers.

38. The compound or pharmaceutically acceptable salt, solvate, ester, amide, or prodrug of the compound of claim 36, wherein the compound is a (Z)-isomer and has the structure

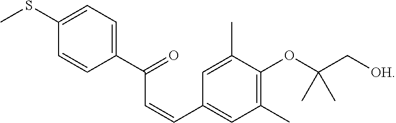 [SMILES: CSc1ccc(C(=O)/C=C\c2cc(C)c(OC(C)(C)CO)c(C)c2)cc1]

39. The compound or pharmaceutically acceptable salt, solvate, ester, amide, or prodrug of the compound of claim 36, wherein the compound is a (E)-isomer and has the structure

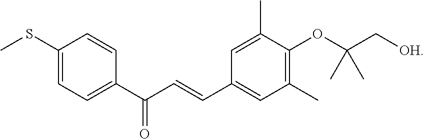 [SMILES: CSc1ccc(C(=O)/C=C/c2cc(C)c(OC(C)(C)CO)c(C)c2)cc1]

40. The compound or pharmaceutically acceptable salt, solvate, ester, amide, or prodrug of the compound of claim 38 or 39, wherein the compound is substantially free of its corresponding other olefin configuration.

41. A compound having the structure: 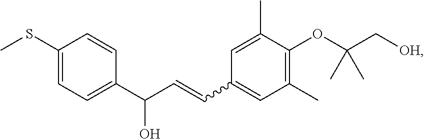 [SMILES: CSc1ccc(C(O)C=Cc2cc(C)c(OC(C)(C)CO)c(C)c2)cc1]
or a pharmaceutically acceptable salt, solvate, ester, amide, or prodrug thereof.

42. The compound or pharmaceutically acceptable salt, solvate, ester, amide, or prodrug of the compound of claim 41, wherein the compound is a racemate or a mixture of enantiomers.

43. The compound or pharmaceutically acceptable salt, solvate, ester, amide, or prodrug of the compound of claim 41, wherein the compound has an hydroxyl-bearing allylic carbon atom having an (R)-stereochemistry and has the structure

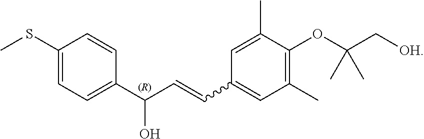

44. The compound or pharmaceutically acceptable salt, solvate, ester, amide, or prodrug of the compound of claim 41, wherein the compound has an hydroxyl-bearing allylic carbon atom having an (S)-stereochemistry and has the structure

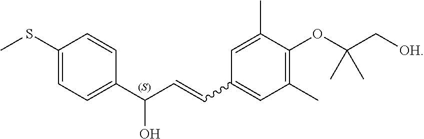

45. The compound or pharmaceutically acceptable salt, solvate, ester, amide, or prodrug of the compound of claim 43 or 44, wherein the compound is substantially free of its corresponding opposite enantiomer.

46. The compound or pharmaceutically acceptable salt, solvate, ester, amide, or prodrug of the compound of any one of claims 43-45, wherein the compound has an olefin isomer configuration of (Z) or (E).

47. The compound or pharmaceutically acceptable salt, solvate, ester, amide, or prodrug of the compound of claim 41, wherein the compound is a (Z)-isomer and has the structure

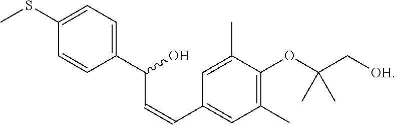 [SMILES: CSc1ccc(C(O)/C=C\c2cc(C)c(OC(C)(C)CO)c(C)c2)cc1]

48. The compound or pharmaceutically acceptable salt, solvate, ester, amide, or prodrug of the compound of claim 41, wherein the compound is a (E)-isomer and has the structure

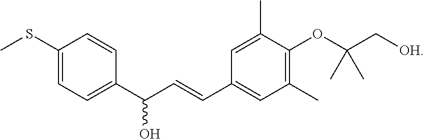 [SMILES: CSc1ccc(C(O)/C=C/c2cc(C)c(OC(C)(C)CO)c(C)c2)cc1]

49. The compound or pharmaceutically acceptable salt, solvate, ester, amide, or prodrug of the compound of claim 47 or 48, wherein the compound is substantially free of its corresponding other olefin configuration.

50. The compound or pharmaceutically acceptable salt, solvate, ester, amide, or prodrug of the compound of any one of claims 47-49, wherein the compound has an hydroxyl-bearing allylic carbon atom having an (R)- or an (S)-stereochemistry.

51. A composition comprising an effective amount of the compound or pharmaceutically acceptable salt, solvate, ester, amide, or prodrug of the compound of any one of claims 1-50 and a pharmaceutically acceptable carrier of vehicle.

52. The composition of claim 51, further comprising another therapeutically active agent.

53. The composition of claim 52, wherein the other therapeutically active agent is a lipid lowering drug, statin, a cholesterol absorption inhibitor, an antibody against PCSK9, an siRNA PCSK9, an anti-fibrotic agent, a thyroid hormone, a selective thyroid receptor-β agonist, apoptosis signal-regulating kinase 1 (ASK1) inhibitor, acetyl-CoA carboxylase (ACC) inhibitor, an integrin antagonist, or a non-steroidal Farnesoid X receptor (FXR) agonist.

54. The composition of claim 53, wherein:
the lipid lowering drug is gemfibrozil, fenofibrate, bezafibrate, clofibrate, ciprofibrate, clinofibrate, etofylline, pirifibrate, simfibrate, tocofibrate, or pemafibrate;
the statin is atorvastatin, simvastatin, pravastatin, rosuvastatin, fluvastatin, lovastatin, pitavastatin, mevastatin, dalvastatin, dihydrocompactin, or cerivastatin, or a pharmaceutically acceptable salt, solvate, ester, amide, or prodrug thereof;
the cholesterol absorption inhibitor is ezetimibe;
the antibody against PCSK9 is evolocumab alirocumab, bococizumab, 1 D05-IgG2, RG7652, LY3015014, or LGT-209;
the siRNA PCSK9 is inclisiran;
the anti-fibrotic agent is nitazoxamide, tizoxanide, or tizoxanide glucuronide, or a pharmaceutically acceptable salt, solvate, ester, amide, or prodrug thereof;
the selective thyroid receptor-β agonist is VK2809, MGL-3196, MGL-3745, SKL-14763, sobetirome, BCT304, ZYT1, MB-0781, or eprotirome;
the ASK1 inhibitor is selonsertib;
the ACC inhibitor is firsocostat;
the integrin antagonist is an α5β1 inhibitor or a pan integrin inhibitor; or
the FXR agonist is cilofexor.

55. A method for treating or preventing a liver disorder, dyslipidemia, dyslipoproteinemia, a renal disease, a disorder of glucose metabolism, a disorder of lipid metabolism, a disorder of glucid metabolism, a cardiovascular disease, a vascular disease, a metabolic syndrome, a complication associated with metabolic syndrome, a PPAR-associated disorder, septicemia, a thrombotic disorder, obesity, diabetic nephropathy, diabetic retinopathy, atherosclerosis, pancreatitis, a cerebrovascular disease, a disorder related to neovascularization, hypertension, cancer, inflammation, an inflammatory disease, a neurodegenerative disease, an autoimmune disease, a neoplastic disease, muscle atrophy, cholestasis, mitochondrial dysfunction, an ocular disease, a lysosomal storage disease, a kidney disease, or impotence, comprising administering to a subject in need thereof an effective amount of the compound or the pharmaceutically acceptable salt, solvate, ester, amide, or prodrug of the compound of any one of claims 1-50.

56. The method of claim 55, wherein the liver disorder involves pathological disruption, inflammation, degeneration, apoptosis, or proliferation of liver cells.

57. The method of claim 55, wherein the liver disorder is liver fibrosis, fatty liver disease, non-alcoholic fatty liver disease (NAFLD) or non-alcoholic steatohepatitis (NASH).

58. The method of claim 55, wherein the dyslipidemia is hyperlipidemia or an abnormally low concentration of high density lipoprotein cholesterol (HDL-C) in the subject's blood plasma or blood serum.

59. The method of claim 58, wherein the hyperlipidemia is hypercholesterolemia, familial hypercholesterolemia, hypertriglyceridemia, or familial combined hyperlipidemia.

60. The method of claim 58, wherein the hyperlipidemia is characterized by: an abnormally reduced or deficient lipoprotein lipase level or activity in the subject's blood plasma or blood serum, or an abnormally high concentration of ketone bodies, lipoprotein(a) cholesterol (Lp(a)-C), low density lipoprotein (LDL), very low density lipoproteins cholesterol (VLDL-C) or non-esterified fatty acids in the subject's blood plasma or blood serum.

61. The method of claim 60, wherein the reduced or deficient lipoprotein lipase level or activity is a result of a mutation in a gene encoding a lipoprotein lipase.

62. The method of claim 55, wherein the dyslipoproteinemia is characterized by an abnormally high concentration of LDL, apolipoprotein (a) or VLDL in a subject's blood plasma or blood serum, or an abnormally low concentration of high density lipoprotein (HDL) or lipoprotein lipase in a subject's blood plasma or blood serum.

63. The method of claim 62, wherein the abnormally low concentration of the lipoprotein lipase is associated with: a lipoprotein lipase mutation, hypoalphalipoproteinemia, a lipoprotein abnormality associated with diabetes, a lipoprotein abnormality associated with obesity, a lipoprotein abnormality associated with Alzheimer's disease, or familial combined hyperlipidemia.

64. The method of claim 55, wherein the renal disease is a glomerular disease, a tubular disease, a tubulointerstitial disease, acute or rapidly progressive renal failure, chronic renal failure, nephrolithiasis, or a tumor.

65. The method of claim 64 wherein:
the glomerular disease is an acute glomerulonephritis, a chronic glomerulonephritis, a rapidly progressive glomerulonephritis, a nephrotic syndrome, a focal proliferative glomerulonephritis, a glomerular lesion associated with systemic disease, Goodpasture syndrome, multiple myeloma, diabetes, neoplasia, sickle cell disease or a chronic inflammatory disease;
the tubular disease is an acute tubular necrosis, an acute renal failure, a polycystic renal disease, medullary sponge kidney, a medullary cystic disease, nephrogenic diabetes, or a renal tubular acidosis;
the tubulointerstitial disease is pyelonephritis, a drug- or toxin-induced tubulointerstitial nephritis, a hypercalcemic nephropathy, or a hypokalemic nephropathy; or
the tumor is renal cell carcinoma or nephroblastoma.

66. The method of claim 65, wherein the glomerular lesion associated with systemic disease is systemic lupus erythematosus.

67. The method of claim 55, wherein the renal disease is hypertension, nephrosclerosis, microangiopathic hemolytic anemia, atheroembolic renal disease, diffuse cortical necrosis, or a renal infarct.

68. The method of claim 67, wherein the hypertension is an essential hypertension, hyperpiesa, hyperpiersis, a malignant hypertension, a secondary hypertension, or a white-coat hypertension.

69. The method of claim 55, wherein the disorder of glucose metabolism is an impaired glucose tolerance; an insulin resistance; an insulin resistance-related breast, colon or prostate cancer; diabetes; pancreatitis; hypertension; polycystic ovarian disease; or an abnormally high concentration of blood insulin or glucose in the subject's blood plasma or blood serum.

70. The method of claim 69, wherein the diabetes is non-insulin dependent diabetes mellitus (NIDDM), insulin dependent diabetes mellitus (IDDM), gestational diabetes mellitus (GDM), or maturity onset diabetes of the young (MODY).

71. The method of claim 55, wherein the vascular disease or the cardiovascular disease is a peripheral vascular disease, a coronary heart disease, stroke, restenosis, arteriosclerosis, ischemia, an endothelium dysfunction, an ischemia-reperfusion injury, a myocardial infarction, or a cerebral infarction.

72. The method of claim 55, wherein the PPAR-associated disorder is rheumatoid arthritis, multiple sclerosis, psoriasis, an inflammatory bowel disease, breast cancer, colon cancer, or prostate cancer.

73. The method of claim 55, wherein the PPAR-associated disorder is a vascular disease, a muscular disease, a demyelinating disease, a muscle structure disorder, a neuronal activation disorder, a muscle fatigue disorder, a muscle mass disorder, a mitochondrial disease, a mitochondrial dysfunction, a beta oxidation disease, or a metabolic disease.

74. The method of claim 73, wherein:
the muscular disease is a muscular dystrophy disease;
the demyelinating disease is multiple sclerosis, Charcot-Marie-Tooth disease, Pelizaeus-Merzbacher disease, encephalomyelitis, neuromyelitis optica, adrenoleukodystrophy, or Guillian-Barre syndrome;
the muscle structure disorder is Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorder, myotonic chondrodystrophy, myotonic dystrophy, myotubular myopathy, nemaline body disease, oculopharyngeal MD, or stress urinary incontinence;
the neuronal activation disorder is amyotrophic lateral sclerosis, Charcot-Marie-Tooth disease, Guillain-Barre syndrome, Lambert-Eaton syndrome, multiple sclerosis, myasthenia gravis, a nerve lesion, peripheral neuropathy, spinal muscular atrophy, tardy ulnar nerve palsy, or toxic myoneural disorder;
the muscle fatigue disorder is chronic fatigue syndrome, diabetes (type I or II), a glycogen storage disease, fibromyalgia, Friedreich's ataxia, intermittent claudication, lipid storage myopathy, MELAS (mitochondrial encephalopathy, lactic acidosis, and stroke-like episodes) syndrome, mucopolysaccharidosis, Pompe disease, or thyrotoxic myopathy;
the muscle mass disorder is cachexia, cartilage degeneration, cerebral palsy, compartment syndrome, critical illness myopathy, inclusion body myositis, muscular atrophy (disuse), sarcopenia, steroid myopathy, or systemic lupus erythematosus;
the mitochondrial disease is Alpers's disease, chronic progressive external ophthalmoplegia (CPEO), Kearns-Sayra syndrome (KSS), Leber hereditary optic neuropathy (LHON), MELAS, myoclonic epilepsy and ragged-red fiber disease (MERRF), neurogenic muscle weakness (NARP), ataxia, retinitis pigmentosa, Pearson syndrome, mitochondrial malfunction, or a mitochondrial loss of functionality;
the beta oxidation disease is systemic camitine transporter, camitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, or riboflavin-responsive disorders of β-oxidation (RR-MADD); or
the metabolic disease is hyperlipidemia, dyslipidemia, hyperchlolesterolemia, hypertriglyceridemia, HDL hypocholesterolemia, LDL hypercholesterolemia, HLD non-cholesterolemia, VLDL hyperproteinemia, dyslipoproteinemia, apolipoprotein A-I hypoproteinemia, atherosclerosis, a disease of arterial sclerosis, a disease of cardiovascular system, cerebrovascular disease, peripheral circulatory disease, metabolic syndrome, syndrome X, obesity, diabetes, type I diabetes, type II diabetes, hyperglycemia, insulin resistance, impaired glucose tolerance, hyperinsulinism, a diabetic complication, cardiac insufficiency, cardiac infarction, cardiomyopathy, hypertension, non-alcoholic fatty liver disease (NAFLD), non-alcoholic steatohepatitis (NASH), a thrombus, Alzheimer disease, a neurodegenerative disease, a demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, or pancreatitis.

75. The method of claim 74, wherein the muscular dystrophy disease is Duchenne muscular dystrophy, Becker muscular dystrophy, a limb-girdle muscular dystrophy, congenital muscular dystrophy, facioscapulohumeral muscular dystrophy, myotonic muscular dystrophy, oculopharyngeal muscular dystrophy, distal muscular dystrophy, or Emery-Dreifuss muscular dystrophy.

76. The method of claim 55, wherein the PPAR-associated disorder is an abnormally low concentration of HDL, an abnormally low concentration of apolipoprotein A-I (apo A-I), an abnormally high concentration of VLDL-C, an abnormally high concentration of low density lipoprotein cholesterol (LDL-C), an abnormally high concentration of triglyceride, an abnormally high concentration of apolipoprotein B (apo B), an abnormally high concentration of apolipoprotein C-III (apo C-III) or an abnormally reduced ratio of post-heparin hepatic lipase to lipoprotein lipase activity in the subject's blood plasma or blood serum.

77. The method of claim 55, wherein the PPAR-associated disorder is an abnormally high concentration of HDL or an abnormally low concentration of apo A-I in the subject's lymph or cerebral fluid.

78. The method of claim 55, wherein the obesity is abdominal obesity.

79. The method of claim 55, wherein the cerebrovascular disease is cerebral ischemia.

80. The method of claim 55, wherein, the disorder related to neovascularization is retinopathy or diabetes.

81. The method of claim 55, wherein the cancer is a human sarcoma or human carcinoma.

82. The method of claim 55, wherein the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, bone cancer, breast cancer, ovarian cancer, prostate cancer, esophageal cancer, oral cancer, nasal cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, leukemia, acute lymphoblastic B-cell leukemia, acute lymphoblastic T-cell leukemia, acute promyelocytic leukemia, acute monoblastic leukemia, acute erythroleukemic leukemia, acute megakaryoblastic leukemia, acute myelomonocytic leukemia, acute nonlymphocytic leukemia, acute undifferentiated leukemia, chronic myelocytic leukemia, hairy cell leukemia, lymphoblastic leukemia, myelogenous leukemia, lymphocyticleukemia, myelocytic leukemia, polycythemia vera, multiple myeloma, lymphoma, Hodgkin's disease, non-Hodgkin's lymphoma, Waldenstrom's macroglobulinemia, or a heavy chain disease.

83. The method of claim 82, wherein the leukemia is acute or chronic lymphoblastic leukemia, myelogenous leukemia, lymphocyticleukemia, lymphocytic leukemia, or myelocytic leukemia.

84. The method of claim 83, wherein the myelocytic leukemia is acute and is myeloblastic, promyclocytic, myelomonocytic, monocytic or erythroleukemia.

85. The method of claim 83, wherein the lymphoma is Hodgkin's lymphoma or non-Hodgkin's lymphoma.

86. The method of claim 55, wherein the inflammatory disease is multiple sclerosis, a chronic inflammatory disorder of a joint, arthritis, a respiratory distress syndrome, an inflammatory bowel disease, an inflammatory lung disorder, an inflammatory disorder, an inflammatory disorder of the gum, tuberculosis, leprosy, an inflammatory disease of the kidney, an inflammatory disorder of the skin, an inflammatory disease of the central nervous system, a systemic lupus erythematosus (SLE) or an inflammatory disease of the heart.

87. The method of claim 86, wherein:
the arthritis is rheumatoid arthritis or osteoarthritis;
the inflammatory bowel disease is ileitis, ulcerative colitis or Crohn's disease;
the inflammatory lung disorder is asthma or chronic obstructive airway disease;
the inflammatory disorder of the eye is corneal dystrophy, trachoma, onchocerciasis, uveitis, sympathic ophthalmitis or endophthalmitis;
the inflammatory disorder of the gum is periodontitis or gingivitis;
the inflammatory disease of the kidney is glomerulonephritis or nephrosis;
the inflammatory disorder of the skin is acne, sclerodermatitis, psoriasis, eczema, photoaging or wrinkles;
the inflammatory disease of the central nervous system is AIDS-related neurodegeneration, stroke, neurotrauma, Alzheimer's disease, encephalomyelitis, or viral or autoimmune encephalitis; or
the inflammatory disease of the heart is cardiomyopathy.

88. The method of claim 55, wherein the neurodegenerative disease is Alzheimer's disease or Huntington's disease.

89. The method of claim 55, wherein the autoimmune disease is immune-complex vasculitis, systemic lupus or erythematodes.

90. The method of claim 55, wherein, the neoplastic disease is carcinogenesis.

91. The method of claim 55, wherein the cholestasis is intrahepatic cholestatic disease or extrahepatic cholestatic disease.

92. The method of claim 91, wherein intrahepatic cholestatic disease is primary biliary cholangitis (PBC), primary sclerosing cholangitis (PSC), progressive familial intrahepatic cholestasis (PFIC), or Alagille syndrome (AS).

93. The method of claim 55, wherein the ocular disease is dry eye, meibomian gland dysfunction, a keratoconjunctiva epithelial disorder, a corneal epithelial disorder, or a corneal ulcer.

94. The method of claim 55, wherein the lysosomal storage disorder is neuronal ceroid lipofuscinosis, Alzheimer's disease, Huntington's disease, amyotrophic lateral sclerosis (ALS), Parkinson's disease, multiple system atrophy (MSA), progressive supranuclear palsy (PSP), corticobasal degeneration (CBD), dementia with Lewy bodies (DLB), a disorder of the autophagy pathway, Tay-Sach's disease, Fabry disease, Niemann-Pick disease, Gaucher disease, Hunter syndrome, alpha-mannosidosis, aspartylglucosaminuria, cholesteryl ester storage disease, chronic hexosaminidase A deficiency, cystinosis, Danon disease, Farber disease, fucosidosis, galactosialidosis, or Batten disease.

95. The method of claim 55, wherein the kidney disease is renal ischemia reperfusion injury.

96. The method of claim 55, wherein the impotence results from damages to a nerve, artery, a smooth muscle, or fibrous tissue; diabetes; kidney disease; alcoholism; multiple sclerosis; atherosclerosis; vascular disease; or neurologic disease.

97. A method for treating or preventing hyperlipemia, hyperlipidemia, hyperlipoproteinemia, hypercholesterolemia, hypertriglyceridemia, or dyslipidemia, comprising administering to a subject in need thereof an effective amount of the compound or the pharmaceutically acceptable salt, solvate, ester, amide, or prodrug of the compound of any one of claims 1-50.

98. The method of claim 97, wherein the hypercholesterolemia is homozygous familial hypercholesterolemia.

99. A method for treating a subject having or preventing a subject from having an abnormally high concentration in a subject's blood plasma or blood serum of high low-density lipoprotein (LDL), apolipoprotein B (apo B), lipoprotein(a) (Lp(a)), apolipoprotein (a), or very low-density lipoprotein (VLDL), comprising administering to a subject in need thereof an effective amount of the compound or the pharmaceutically acceptable salt, solvate, ester, amide, or prodrug of the compound of any one of claims 1-50.

100. A method for treating a subject having or preventing a subject from having an abnormally low concentration in a subject's blood plasma or blood serum of high-density lipoprotein (HDL), comprising administering to a subject in need thereof an effective amount of the compound or the pharmaceutically acceptable salt, solvate, ester, amide, or prodrug of the compound of any one of claims 1-50.

101. A method for treating a subject having or preventing a subject from having an abnormally reduced or deficient lipoprotein lipase concentration or activity in a subject's blood plasma or blood serum, comprising administering to a subject in need thereof an effective amount of the compound or the pharmaceutically acceptable salt, solvate, ester, amide, or prodrug of the compound of any one of claims 1-50.

102. The method of claim 101, wherein the reduced or deficient lipoprotein lipase level or activity is a result of a mutation in a gene encoding a lipoprotein lipase.

103. A method for treating or preventing hypoalphalipoproteinemia, a lipoprotein abnormality associated with diabetes, a lipoprotein abnormality associated with obesity, a lipoprotein abnormality associated with Alzheimer's Disease, or familial combined hyperlipidemia, comprising administering to a subject in need thereof an effective amount of the compound or the pharmaceutically acceptable salt, solvate, ester, amide, or prodrug of the compound of any one of claims 1-50.

104. A method for reducing in a subject's blood plasma or blood serum an abnormally high concentration of triglyceride, low-density lipoprotein cholesterol (LDL-C), very low-density lipoprotein cholesterol (VLDL-C), non-high-density lipoprotein cholesterol, (non-HDL-C), lipoprotein(a) (Lp(a)), apolipoprotein B, HDL/(VLDL+LDL) ratio, apolipoprotein C-II or apolipoprotein C-III, comprising administering to a subject in need thereof an effective amount of the compound or the pharmaceutically acceptable salt, solvate, ester, amide, or prodrug of the compound of any one of claims 1-50.

105. A method for elevating in a subject's blood plasma or blood serum an abnormally low concentration of a high-density lipoprotein (HDL)-associated protein, HDL-cholesterol, apolipoprotein A-I, or apolipoprotein E, comprising administering to a subject in need thereof an effective amount of the compound or the pharmaceutically acceptable salt, solvate, ester, amide, or prodrug of the compound of any one of claims 1-50.

106. The method of claim 105, wherein the HDL-associated protein is apolipoprotein A-I (apo A-I), apolipoprotein A-II (apo A-II), apolipoprotein A-IV (apo A-IV) or apolipoprotein E (apo E).

107. A method for promoting clearance of triglyceride from a subject's blood plasma or blood serum, comprising administering to a subject in need thereof an effective amount of the compound or the pharmaceutically acceptable salt, solvate, ester, amide, or prodrug of the compound of any one of claims 1-50.

108. A method for increasing abnormally low glucose metabolism or lipid metabolism in a subject, comprising administering to a subject in need thereof an effective amount of the compound or the pharmaceutically acceptable salt, solvate, ester, amide, or prodrug of the compound of any one of claims 1-50.

109. The method of claim 108, wherein the method for increasing abnormally low glucose metabolism increases insulin sensitivity or oxygen consumption of a subject or decreases blood insulin, blood glucose, or glycated hemoglobin in a subject's blood plasma or blood serum.

110. The method of claim 108, wherein the method for increasing abnormally low lipid metabolism reduces a concentration of LDL or free triglyceride in a subject's blood plasma or blood serum, or inhibits saponified or non-saponified fatty acid synthesis.

111. A method for treating or preventing a symptom of a disease selected from inflammation, systemic lupus erythematosus, lupus nephritis, or arthritis, comprising administering to a subject in need thereof an effective amount of the compound or the pharmaceutically acceptable salt, solvate, ester, amide, or prodrug of the compound of any one of claims 1-50.

112. The method of claim 111, wherein the arthritis is adjuvant arthritis or type II collagen-induced arthritis.

113. The method of claim 111, wherein the symptom is nephritis, kidney failure, or kidney function reduction.

114. A method for reducing the fat content of meat in livestock, comprising administering to livestock an effective amount of the compound or the pharmaceutically acceptable salt, solvate, ester, amide, or prodrug of the compound of any one of claims 1-50.

115. A method for reducing cholesterol content of a fowl egg, comprising administering to a fowl species an effective amount of the compound or the pharmaceutically acceptable salt, solvate, ester, amide, or prodrug of the compound of any one of claims 1-50.
